SEQUENCE LISTING (106 sequences)

SEQ ID NO: 1 - protein, 283 aa, Petunia hybrida
MEKQTQARFLEPWRDLKGKVVMVTGASSGIGLDLCLDLAKAGCKIIAAARRVDRLKSLCNQINAAADDDDDNNRAVAVELDVSANGDAIEAAVKEAWGAFGRIDALVNNAGVRGTVHSPLELTEEECHNTFRTNLTGTWLVSKYVCKFMRDAKQGGSVINISSIAGLNRGQLPGGLAYVSSKGAVNMVTKVMALELGVDKIRVNSISPGLFKSEITQGLIQKEWLNNVAMRTIPLRTHGNSADPALTSVVRYLIHDSSEYVSGNLFIVDAGATLPGVPIFSSL

SEQ ID NO: 2 - protein, 266 aa, Petunia hybrida [sequence omitted]

SEQ ID NO: 3 - DNA, 852 bp, Petunia hybrida
  source 1..852: mol_type = genomic DNA
atggagaagcaaactcaggcacgtttcttggaaccatggagagacttgaaaggaaaagttgtgatggtcacgggtgcatcatcaggaattggtctagatttatgtctagacttagctaaagctggttgtaaaatcattgctgctgctcgtcgtgtcgatcgacttaaatcactctgtaatcaaatcaatgctgctgctgatgatgatgatgataacaaccgtgctgttgctgttgagcttgatgttagtgctaatggtgacgccattgaagctgctgttaaagaagcttggggtgcttttggacgtatcgatgctttggttaataacgctggtgttagaggtactgtgcactctccgttggaattgacagaggaggagtgccataatacctttagaacaaacctaacggggacatggttggtgtccaaatatgtttgtaaattcatgcgtgatgctaaacaaggaggatctgttattaatatctcttcaattgctggtcttaatcgtgggcaattaccaggtggtcttgcttatgtttcttcaaagggtgctgttaacatggtgacaaaggtgatggctcttgaacttggagtagacaaaatcagagtgaactcaatatctcccgggcttttcaaatctgagataacacagggcctcattcaaaaagaatggctcaacaatgttgctatgagaaccattcctctgagaactcatggaaattcagctgatccagctttaacatcagtagtccgatacttgattcacgattcttcagaatatgtatcaggcaatctttttattgtcgatgctggtgctaccttaccaggtgtcccgattttctcatccctgtag

SEQ ID NO: 4 - DNA, 801 bp, Petunia hybrida
  source 1..801: mol_type = genomic DNA
atggaaaattctggaaagaaggtgttgcttacttcaaatggcgatgacatttgcaataacattgcttaccatttagctcagaggggttgccagttggttttgatgggaaatgagagccaattgaagagtgtagctgagatgataaagcaatctctaggtggtagcgctgcagtggaagttgtgggtttggatatggaggaggatagagaagctgcttttgaggaagctgtggacaaggcatggaagatatttggaaagctggatgctttggtaaactgctattcatatgaaggaaaaatgcaagatccactgcagctaattgaagaagagttcaaaaagattgtcaaaataaattttatggctggatggtacctgatgaaatgtatcagcaaaaaaatgcgagatgataaatcagggggatccattgtattcttgacctcaattattggtgcggagagaggaatatatcaaggagctgctgcttatggttcatgtctgggtggaatacagcagctggtcaggctagctgcaatagagctggggaagcaccaaattagggtgaatggcatatctcgtggcttgcacctccaggatgagtatcctaaggcggtgggattggagagagcagagaagatgaccaaggaagcagcgcctctaaatcgatggcttgatgttaaaaaggatctggcatccactgtcatctatttaatcagtgatgattcaagatacatgactgggacctccatctttgttgatggtgcccagtctctagtaaggccgcgaatgcgctcatacatgtga

SEQ ID NO: 5 - protein, 293 aa, Arabidopsis thaliana
MANQVADHLEPWRHLHGKVVMVTGASSGLGREFCLDLAKAGCRIVAAARRIDRLQSVCDEINQLTVTAPSSSSSSSSVNGPGSSRAVAVELDISADGPAIEKSVNKAWDAFGRIDALVNNAGVRGNVSSSLELSEEEWNNVFKTNLTGSWLVSKYVSIRMRDADQGGSIINVSSIAGLHRGYLPGAVAYNCSKAGVNTLAKVMAMELGVHKIRVNAISPGLFRSEITEGLMKKDWLHNVAMKTVPLRTFGTSDPALTSLVRYLVHDSSEYVSGNVYIVDAGATLPGVPIFSAL

SEQ ID NO: 6 - protein, 293 aa, Prunus dulcis; identical to SEQ ID NO: 5

SEQ ID NO: 7 - protein, 287 aa, Solanum lycopersicum
METHAKSVRQLEPWGELAGKVVMVTGASSGIGRDFCSDLSKAGCRIIAAARRIDRLQSLCDEINSNSSNGSTKSSQDLRAVAIELDVSANGSAIEAAVQKAWDAFGRIDGLVNNAGFRGSVLSPLELSEEEWEKIHKTNLRGAWLVTKYVCMHMRAANQGGSIINISSIAGINRGQLPGSLAYTSSKEALNSITKVLALELGPYKIRVNSISPGLFKSEITEGLIQKDWIKNIELRTIPLRTHGTSHPALTSVVRYLIHDSSEYVSGNMFIVDAGATLPGVPIFSSL

SEQ ID NO: 8 - protein, 266 aa, Arabidopsis thaliana
MENPAKRVLMTSNGDEVSRNIAFHLAKHGCKLVMMGNEGSLRSIVDKIRDSIEGAFPADVIALDMESDSEVAFHAAVQKAWELSGHFDAFLNSYTYQGKVQDILQVSQDEFHRITKINLTAPWFLLKAVATRMKDHGSGGSIVFMATIASGERALYPGADAYASTSAAIHQLVRASAMSLGKHKIRVNMISRGLHLDDEYTASVGRDRAQKLVKDAAPLGQWLNPDTDLYSTVIYLISDGSRFMTGTTVLVDGAQSLTRPRLKSYM

SEQ ID NO: 9 - protein, 267 aa, Prunus dulcis
MGTSPGKKVLLTSNGDAISHNIAFSLAQRGCRLVLMGKESCLRSIQQKIKGSLEGVVPVEVVDVDMEDKREGAFDEAVDKACHILGNLDAFVHCYTYEGKMQEHLELAEDEFKKIMKRNFMSAWFLLNAVGRRMRDYKSGGSIIFLTSIIGAERGIYPGAAANSACSAAVQQLARTSALEIGRYQIRVNAIARGLHLEDEYPMFVGMERAKKLVKEAAPLQRWLDVKNDLASTVIYLISDGSKYMTGTTIFVDGGQSLTRPRMRSYM

SEQ ID NO: 10 - protein, 266 aa, Solanum lycopersicum
MENPGKKVLLTSNGDEICNNIAYHLAQRGCQLVLMGNERQLKSVAENIKQSLKGSVAVEVVGLDMTEDRETAFDEAVDKAWKIFGKLDALVHCYAYEGKMQDPLQLIDDEFKKIVKINFMAGWYLLKCIGNRMRDGKSGGSIVFMTSIIGAERGIYQGAAAYGSCAAGIQQLVRLSAIELGKYQIRVNGILRGLHLEDEFPLSVGKERAVKLTKEAAPLNRWLDPKKDLASTVIYLISDDSRYMTGTSIFVDGAQSLVRPRMRSYM

SEQ ID NO: 11 - DNA, 300 bp, Synthetic construct
  source 1..300: mol_type = other DNA
atgatatttgcgatgcctaacaagccaggggagttcagccgctttgattttcctgaagctcttcctgcgccattaaatggaattttggccatactaaagaacaacgaaatgcttacgtggcccgagaaagtcaaatttgctattggactcttgccagcaatgcttggagggcaatcttatgttgaagctcaagacggtttaagtgttaaggactggatgagaaagcaaggtgtgcctgatagggtgacagatgaggtgttcattgccatgtcaaaggcacttaacttcataaaccctgac

SEQ ID NO: 12 - protein, 283 aa, Petunia hybrida; identical to SEQ ID NO: 1

SEQ ID NO: 13 - protein, 267 aa, Petunia hybrida
MENSGKKVLLTSNGDDICNNIAYHLAQRGCQLVLMGNESQLKSVAEMIKQSLGGSAAVEVVGLDMEEDREAAFEEAVDKAWKIFGKLDALVNCYSYEGKMQDPLQLIEEEFKKIVKINFMAGWYLMKCISKKMRDDKSGGSIVFLTSIIGAERGIYQGAAAYGSCLGGIQQLVRLAAIELGKHQIRVNGISRGLHLQDEYPKAVGLERAEKMTKEAAPLNRWLDVKKDLASTVIYLISDDSRYMTGTSIFVDGAQSLELFAKLVARA

SEQ ID NO: 14 - DNA, 37 bp, Synthetic construct
  source 1..37: mol_type = other DNA
atccggtaccgagctcggagaagcaaactcaggcacg

SEQ ID NO: 15 - DNA, 24 bp, Synthetic construct
  source 1..24: mol_type = other DNA
agaattttcctccaaaagcacccc

SEQ ID NO: 16 - DNA, 29 bp, Synthetic construct
  source 1..29: mol_type = other DNA
tgcttttggaggaaaattctggaaagaag

SEQ ID NO: 17 - DNA, 50 bp, Synthetic construct
  source 1..50: mol_type = other DNA
cgagacgcgtgagctcttgcatttttccttcatatgaatagcagtttacc

SEQ ID NO: 18 - DNA, 42 bp, Synthetic construct
  source 1..42: mol_type = other DNA
gcgatatcgtcgacggatccatggagaagcaaactcaggcac

SEQ ID NO: 19 - DNA, 46 bp, Synthetic construct
  source 1..46: mol_type = other DNA
cctgcagggaattcggatccctacagggatgagaaaatcgggacac

SEQ ID NO: 20 - DNA, 47 bp, Synthetic construct
  source 1..47: mol_type = other DNA
gcgatatcgtcgacggatccatggaaaattctggaaagaaggtgttg

SEQ ID NO: 21 - DNA, 42 bp, Synthetic construct
  source 1..42: mol_type = other DNA
cctgcagggaattcggatcctcacatgtatgagcgcattcgc

SEQ ID NO: 22 - DNA, 49 bp, Synthetic construct
  source 1..49: mol_type = other DNA
ctttaagaaggagatatacatatggaaaattctggaaagaaggtgttgc

SEQ ID NO: 23 - DNA, 40 bp, Synthetic construct
  source 1..40: mol_type = other DNA
tcgagtgcggccgcaagcttcatgtatgagcgcattcgcg

SEQ ID NO: 24 - DNA, 45 bp, Synthetic construct
  source 1..45: mol_type = other DNA
gcgatatcgtcgacggatccatgagcaatcatcaaactcaggtgt

SEQ ID NO: 25 - DNA, 48 bp, Synthetic construct
  source 1..48: mol_type = other DNA
cctgcagggaattcggatcctcagagagatgaaaagataggcacaccg

SEQ ID NO: 26 - DNA, 42 bp, Synthetic construct
  source 1..42: mol_type = other DNA
gcgatatcgtcgacggatccatggaaaatccggcgaagagag

SEQ ID NO: 27 - DNA, 45 bp, Synthetic construct
  source 1..45: mol_type = other DNA
cctgcagggaattcggatcctcacatgtaggatttgagacggggt

SEQ ID NO: 28 - DNA, 42 bp, Synthetic construct
  source 1..42: mol_type = other DNA
ccatggaggccagtgaattcatggagaagcaaactcaggcac

SEQ ID NO: 29 - DNA, 46 bp, Synthetic construct
  source 1..46: mol_type = other DNA
tgcccacccgggtggaattcctacagggatgagaaaatcgggacac

SEQ ID NO: 30 - DNA, 47 bp, Synthetic construct
  source 1..47: mol_type = other DNA
ccatggaggccagtgaattcatggaaaattctggaaagaaggtgttg

SEQ ID NO: 31 - DNA, 42 bp, Synthetic construct
  source 1..42: mol_type = other DNA
tgcccacccgggtggaattctcacatgtatgagcgcattcgc

SEQ ID NO: 32 - DNA, 44 bp, Synthetic construct
  source 1..44: mol_type = other DNA
tatggccatggaggccgaattcatggagaagcaaactcaggcac

SEQ ID NO: 33 - DNA, 46 bp, Synthetic construct
  source 1..46: mol_type = other DNA
cgacggatccccgggaattcctacagggatgagaaaatcgggacac

SEQ ID NO: 34 - DNA, 49 bp, Synthetic construct
  source 1..49: mol_type = other DNA
tatggccatggaggccgaattcatggaaaattctggaaagaaggtgttg

SEQ ID NO: 35 - DNA, 42 bp, Synthetic construct
  source 1..42: mol_type = other DNA
cgacggatccccgggaattctcacatgtatgagcgcattcgc

SEQ ID NO: 36 - DNA, 43 bp, Synthetic construct
  source 1..43: mol_type = other DNA
tattctgcccaaattcgcgccatggagaagcaaactcaggcac

SEQ ID NO: 37 - DNA, 46 bp, Synthetic construct
  source 1..46: mol_type = other DNA
agctcctcgcccttgctcaccatcagggatgagaaaatcgggacac

SEQ ID NO: 38 - DNA, 48 bp, Synthetic construct
  source 1..48: mol_type = other DNA
tattctgcccaaattcgcgccatggaaaattctggaaagaaggtgttg

SEQ ID NO: 39 - DNA, 43 bp, Synthetic construct
  source 1..43: mol_type = other DNA
agctcctcgcccttgctcaccatcatgtatgagcgcattcgcg

SEQ ID NO: 40 - DNA, 38 bp, Synthetic construct
  source 1..38: mol_type = other DNA
ccacaacatcgaggacatggagaagcaaactcaggcac

SEQ ID NO: 41 - DNA, 56 bp, Synthetic construct
  source 1..56: mol_type = other DNA
aaaatttaatgaaaccagagttaaccatggctacagggatgagaaaatcgggacac

SEQ ID NO: 42 - DNA, 43 bp, Synthetic construct
  source 1..43: mol_type = other DNA
ggacgagctgtacaagatggaaaattctggaaagaaggtgttg

SEQ ID NO: 43 - DNA, 53 bp, Synthetic construct
  source 1..53: mol_type = other DNA
aaaatttaatgaaaccagagttaaccatggtcacatgtatgagcgcattcgcg

SEQ ID NO: 44 - DNA, 40 bp, Synthetic construct
  source 1..40: mol_type = other DNA
ctgcccaaattcgcgatggacgagttaccaaaatgtggag

SEQ ID NO: 45 - DNA, 44 bp, Synthetic construct
  source 1..44: mol_type = other DNA
tttaatgaaaccagagttaactacagacgagctggcaaatcaag

SEQ ID NO: 46 - DNA, 38 bp, Synthetic construct
  source 1..38: mol_type = other DNA
ctgcccaaattcgcgatggctcaagttaaggtgacgat

SEQ ID NO: 47 - DNA, 43 bp, Synthetic construct
  source 1..43: mol_type = other DNA
tttaatgaaaccagagttaattacatgcgtgaccttgaagctg

SEQ ID NO: 48 - DNA, 41 bp, Synthetic construct
  source 1..41: mol_type = other DNA
ctgcccaaattcgcgatggagaaagcaattcaaaggcaaag

SEQ ID NO: 49 - DNA, 51 bp, Synthetic construct
  source 1..51: mol_type = other DNA
tttaatgaaaccagagttaattatttcgcatccttggataagaaactgttg

SEQ ID NO: 50 - DNA, 44 bp, Synthetic construct
  source 1..44: mol_type = other DNA
ctgcccaaattcgcgatggagtatgccaatgaaaactgtaatgg

SEQ ID NO: 51 - DNA, 43 bp, Synthetic construct
  source 1..43: mol_type = other DNA
tttaatgaaaccagagttaattagcagagtggaagaggagcac

SEQ ID NO: 52 - DNA, 42 bp, Synthetic construct
  source 1..42: mol_type = other DNA
ctgcccaaattcgcgatggagaagcaaactcaggcacgtttc

SEQ ID NO: 53 - DNA, 42 bp, Synthetic construct
  source 1..42: mol_type = other DNA
tttaatgaaaccagagttaactacagggatgagaaaatcggg

SEQ ID NO: 54 - DNA, 48 bp, Synthetic construct
  source 1..48: mol_type = other DNA
ctgcccaaattcgcgatggaaaattctggaaagaaggtgttgcttact

SEQ ID NO: 55 - DNA, 42 bp, Synthetic construct
  source 1..42: mol_type = other DNA
tttaatgaaaccagagttaatcacatgtatgagcgcattcgc

SEQ ID NO: 56 - DNA, 30 bp, Synthetic construct
  source 1..30: mol_type = other DNA
tggctgaggctgatgatattcaaccaatcg

SEQ ID NO: 57 - DNA, 30 bp, Synthetic construct
  source 1..30: mol_type = other DNA
ccaacatatgcatccttctggttcatccca

SEQ ID NO: 58 - DNA, 26 bp, Synthetic construct
  source 1..26: mol_type = other DNA
cccattggccatcgaacacaccacac

SEQ ID NO: 59 - DNA, 26 bp, Synthetic construct
  source 1..26: mol_type = other DNA
agctgcggcttggattccagttgaac

SEQ ID NO: 60 - DNA, 28 bp, Synthetic construct
  source 1..28: mol_type = other DNA
ggaaaatccggcgaagagagtgttgatg

SEQ ID NO: 61 - DNA, 27 bp, Synthetic construct
  source 1..27: mol_type = other DNA
tgctataacatcggcagggaaggctcc

SEQ ID NO: 62 - DNA, 20 bp, Synthetic construct
  source 1..20: mol_type = other DNA
cctggtcaaattggaaacgg

SEQ ID NO: 63 - DNA, 21 bp, Synthetic construct
  source 1..21: mol_type = other DNA
cagatcgcctgtcaatcttgg

SEQ ID NO: 64 - DNA, 30 bp, Synthetic construct
  source 1..30: mol_type = other DNA
cactctccgttggaattgacagaggaggag

SEQ ID NO: 65 - DNA, 29 bp, Synthetic construct
  source 1..29: mol_type = other DNA
ccacctggtaattgcccacgattaagacc

SEQ ID NO: 66 - DNA, 30 bp, Synthetic construct
  source 1..30: mol_type = other DNA
cttgacctcaattattggtgcggagagagg

SEQ ID NO: 67 - DNA, 27 bp, Synthetic construct
  source 1..27: mol_type = other DNA
tcctggaggtgcaagccacgagatatg

SEQ ID NO: 68 - DNA, 26 bp, Synthetic construct
  source 1..26: mol_type = other DNA
ccattagttttctattgttacatggc

SEQ ID NO: 69 - DNA, 23 bp, Synthetic construct
  source 1..23: mol_type = other DNA
cactcgacaatacaaaagacacc

SEQ ID NO: 70 - DNA, 24 bp, Synthetic construct
  source 1..24: mol_type = other DNA
aaggcactcaatgtctattttcgg

SEQ ID NO: 71 - DNA, 29 bp, Synthetic construct
  source 1..29: mol_type = other DNA
gaataagataattcaatatgtgcgtgaac

SEQ ID NO: 72 - DNA, 21 bp, Synthetic construct
  source 1..21: mol_type = other DNA
tgcacttcataggtggtcgag

SEQ ID NO: 73 - DNA, 22 bp, Synthetic construct
  source 1..22: mol_type = other DNA
agagagatctgaaaggacccct

SEQ ID NO: 74 - DNA, 25 bp, Synthetic construct
  source 1..25: mol_type = other DNA
actctgttaatgacaaccccttgat

SEQ ID NO: 75 - DNA, 20 bp, Synthetic construct
  source 1..20: mol_type = other DNA
ctgaaaattcccaccatgca

SEQ ID NO: 76 - DNA, 21 bp, Synthetic construct
  source 1..21: mol_type = other DNA
atcgcaacatctccaattcca

SEQ ID NO: 77 - DNA, 20 bp, Synthetic construct
  source 1..20: mol_type = other DNA
caaacgatggctcgatcaca

SEQ ID NO: 78 - DNA, 21 bp, Synthetic construct
  source 1..21: mol_type = other DNA
tgatgcaaaggctgtgttgaa

SEQ ID NO: 79 - DNA, 20 bp, Synthetic construct
  source 1..20: mol_type = other DNA
ttgctgcccttggatcatct

SEQ ID NO: 80 - DNA, 34 bp, Synthetic construct
  source 1..34: mol_type = other DNA
gccttttcagaaatggataaatagccttgcttcc

SEQ ID NO: 81 - DNA, 19 bp, Synthetic construct
  source 1..19: mol_type = other DNA
attttgccgatttcggaac

SEQ ID NO: 82 - DNA, 24 bp, Synthetic construct
  source 1..24: mol_type = other DNA
acatgtcacaatgatttcgtatcc

SEQ ID NO: 83 - DNA, 24 bp, Synthetic construct
  source 1..24: mol_type = other DNA
ccctcaatggaatctctaatcttg

SEQ ID NO: 84 - DNA, 24 bp, Synthetic construct
  source 1..24: mol_type = other DNA
acatgtcacaatgatttcgtatcc

SEQ ID NO: 85 - DNA, 24 bp, Synthetic construct
  source 1..24: mol_type = other DNA
ggaggaaagaagacaaaatgagag

SEQ ID NO: 86 - DNA, 24 bp, Synthetic construct
  source 1..24: mol_type = other DNA
gattaagtacgtacctggggaatg

SEQ ID NO: 87 - DNA, 24 bp, Synthetic construct
  source 1..24: mol_type = other DNA
tatgtttgttgatgattgggtttc

SEQ ID NO: 88 - DNA, 24 bp, Synthetic construct
  source 1..24: mol_type = other DNA
atatgtttggtatttgttgatgcg

SEQ ID NO: 89 - DNA, 24 bp, Synthetic construct
  source 1..24: mol_type = other DNA
gctcatattgttttctctgtgtgg

SEQ ID NO: 90 - DNA, 24 bp, Synthetic construct
  source 1..24: mol_type = other DNA
tcggtattattgaaccattgattg

SEQ ID NO: 91 - DNA, 24 bp, Synthetic construct
  source 1..24: mol_type = other DNA
tgatgaaacgtctaactcaagagc

SEQ ID NO: 92 - DNA, 1426 bp, Arabidopsis thaliana
  source 1..1426: mol_type = genomic DNA
gactaaagtgttaattaggacctagaccctacagatttgggcataaaccgtcctttgttatgggacgtgttctacttggtatctctagaaccgcggtctttggcagatggatcagattatatctttttgacacttgtatttatatatgttatatgttattggcttctccttccccattggccatcgaacacaccacacagagaaaacaatatgagcaatcatcaaactgtgagataacattttctctcatttcccatctttctttttatcttcaaaactattcatataaccatttatataagaccactctccaaattaccatgcttttaacctttaaatatgatttctgttatcttctttaccatatgattccatatggaaaaattgtcaatcatgtgtttttgtctttgtaatctgaaattacttgatttttgtattttgttttttttttgggggagaaaagcaggtgttgaagctattggagccatggtgtgaacttaaagacaaagtggttcttgtgacaggagcttcctctggtattggaagagagatctgtcttgatcttgccaaagctggttgccaggttattgcagcagctcgtcgtgttgatcgactcaactctctctgctctgaaatcaacagcttcagttcaactggaatccaagccgcagctcttgagttagacgtttcatcagacgcagccaccattcaaaaagcggtcagggaagcttgggacatctttggaaagatcgatgcattgatcaacaatgctggaatcagaggcaatgtcaagtcgagtttggatttgtcagaggacgaatgggacaacgtcttcaagaccaacttaaagggtccttggttagtatccaaacatgtttgtatgttaatgcgtgacgctaaacgaggtggctcggtgataaacatctcatcgatcgccgggatccggggtatgttgcccggtggacttgcttatgcttgttccaaaggcggtgttgacaccatgtcaaggatgatggcacttgagttaggtgttcataagatcagagtgaactcgatcgcaccggggcttttcaagtcagagatcacacaaggtcttatgcaaaaggagtggctcaagaatgttaccgagaggactgtgccgttaaaggtgcaacagaccgttgatccagggcttacttctctggttcgctatctcattcatgactcttctcaatatatatccgggaatacatacattgttgattccggtgctacattgcccggtgtgcctatcttttcatctctctgaaaatcgaaccttataatatcttatcttgtgcaataataagtgaatacatttactcattctctactactttttaagatttacatacatcgtcaaaaatttgcaataacaaaccagcagagacacaaaaagaaaggacag

SEQ ID NO: 93 - DNA, 1933 bp, Arabidopsis thaliana
  source 1..1933: mol_type = genomic DNA
atgttgatgattcattgtctgtctggagctcccaaatgccttttatgcttacatattcataaccaacaacggctattaattataaaccaaaaacacgaaataagtttgtagcaaagtgaaattaggaatcttggagatggatccattagtagtaggataataggatatgatggaatttggttggggaacagtgataacttacgcttgcttccggcgccgggaaagttggaaaacctacaaagtacagaaatggatctgggccttgaagtgggctttttattaaagaaaaaaatacatctccgttatcaatcaccatcttcttctatctacaaattaaagaaggtaacaacagaacgtggtggatcatgtggttaggcattaattatttgctttgtttcgccgttttggtaacacacagacacagttccggtaagagcttttgcagccactctttatagttatttagaattggcgatcgaatcaatctcactccctccctcccttaagtcttgttgaatctgctgaattgttttataaagagttactttggcaaaatggaaaatccggcgaagagagtgttgatgacatccaacggcgacgaggtgtcccgaaacatcgctttccatctagccaaacacggttgcaagtgagtctctcattttgtcttctttcctcctatcctgtgtttcttcttctagccccgatttgtgttgtggttgactttggtgtctatagttaaccttcaaaaaatgcaatgtactgttgctaggttggtaatgatgggaaatgagggttccctaaggagcattgtagacaagattagagattccattgagggagccttccctgccgatgttatagcactcgacatggaatctgactctgaagttgcttttcatgccgctgtccaaaaggcatgggaactttccggccatttcgatgcttttctcaactcttatacctaccaaggtttaatttgcttcttgtttttcactaccctgcctttgatgctcttgtgtgttgatcattcctttattcaacaatctttctttcttgcaggaaaggtgcaggacattcttcaagtctctcaagatgagttccacagaatcacaaagatcaatctcaccgctccatggtttctcttgaaggctgtagccacaaggatgaaggaccatggatcaggaggctccattgtcttcatggccactatcgccagcggagagagggcgctttaccctggcgctgatgcctacgcttcaacttctgccgctattcaccagctcgtccgggtatgcatcctagctcctaattagacacatcgcgttcgtaacttgaatatgtttgttgatgattgggtttcaggcatcagccatgagtctcgggaagcacaagatacgggtcaacatgatctctagagggctgcatctggatgatgagtatacagcttctgtgggaagagaccgagcgcagaagctggtcaaggacgctgcacccctcggccagtggctcaacccggacacagacctctactccactgttatctacttgatcagcgatggctcacgcttcatgacaggcaccactgtcttggtggatggagcgcagtcccttacgcgaccccgtctcaaatcctacatgtgatcaatgcctagtattattataattctatgttgtgtgtaaaaagtgaatatgaatcaagtttgaataactatggagggatgaataatccatggaatcaatgattatatacttttgacaagtatagaagaaagcaaagagccaaaatagtcaacaatctcaacatcattcgagctcattgtttattgaattcaatcaagtttggtaggtgaacctcaaatacacgtgatattggatattatcttggaaaccttgctaatccaaattgtaaatcaaataagaaaagaatgattac

SEQ ID NO: 94 - DNA, 25 bp, Synthetic construct
  source 1..25: mol_type = other DNA
tattgaaagaaagtcctgattgctg

SEQ ID NO: 95 - DNA, 25 bp, Synthetic construct
  source 1..25: mol_type = other DNA
tcaataaatgatgaagttttttctc

SEQ ID NO: 96 - DNA, 25 bp, Synthetic construct
  source 1..25: mol_type = other DNA
tcccgtaaaatatcttttactgcat

SEQ ID NO: 97 - protein, 293 aa, Prunus dulcis; identical to SEQ ID NO: 5

SEQ ID NO: 98 - protein, 267 aa, Prunus dulcis; identical to SEQ ID NO: 9

SEQ ID NO: 99 - protein, 287 aa, Solanum lycopersicum; identical to SEQ ID NO: 7

SEQ ID NO: 100 - protein, 266 aa, Solanum lycopersicum; identical to SEQ ID NO: 10

SEQ ID NO: 101 - DNA, 843 bp, Arabidopsis thaliana
  source 1..843: mol_type = genomic DNA
atgagcaatcatcaaactcaggtgttgaagctattggagccatggtgtgaacttaaagacaaagtggttcttgtgacaggagcttcctctggtattggaagagagatctgtcttgatcttgccaaagctggttgccaggttattgcagcagctcgtcgtgttgatcgactcaactctctctgctctgaaatcaacagcttcagttcaactggaatccaagccgcagctcttgagttagacgtttcatcagacgcagccaccattcaaaaagcggtcagggaagcttgggacatctttggaaagatcgatgcattgatcaacaatgctggaatcagaggcaatgtcaagtcgagtttggatttgtcagaggacgaatgggacaacgtcttcaagaccaacttaaagggtccttggttagtatccaaacatgtttgtatgttaatgcgtgacgctaaacgaggtggctcggtgataaacatctcatcgatcgccgggatccggggtatgttgcccggtggacttgcttatgcttgttccaaaggcggtgttgacaccatgtcaaggatgatggcacttgagttaggtgttcataagatcagagtgaactcgatcgcaccggggcttttcaagtcagagatcacacaaggtcttatgcaaaaggagtggctcaagaatgttaccgagaggactgtgccgttaaaggtgcaacagaccgttgatccagggcttacttctctggttcgctatctcattcatgactcttctcaatatatatccgggaatacatacattgttgattccggtgctacattgcccggtgtgcctatcttttcatctctctga

SEQ ID NO: 102 - DNA, 801 bp, Arabidopsis thaliana
  source 1..801: mol_type = genomic DNA
atggaaaatccggcgaagagagtgttgatgacatccaacggcgacgaggtgtcccgaaacatcgctttccatctagccaaacacggttgcaagttggtaatgatgggaaatgagggttccctaaggagcattgtagacaagattagagattccattgagggagccttccctgccgatgttatagcactcgacatggaatctgactctgaagttgcttttcatgccgctgtccaaaaggcatgggaactttccggccatttcgatgcttttctcaactcttatacctaccaaggaaaggtgcaggacattcttcaagtctctcaagatgagttccacagaatcacaaagatcaatctcaccgctccatggtttctcttgaaggctgtagccacaaggatgaaggaccatggatcaggaggctccattgtcttcatggccactatcgccagcggagagagggcgctttaccctggcgctgatgcctacgcttcaacttctgccgctattcaccagctcgtccgggcatcagccatgagtctcgggaagcacaagatacgggtcaacatgatctctagagggctgcatctggatgatgagtatacagcttctgtgggaagagaccgagcgcagaagctggtcaaggacgctgcacccctcggccagtggctcaacccggacacagacctctactccactgttatctacttgatcagcgatggctcacgcttcatgacaggcaccactgtcttggtggatggagcgcagtcccttacgcgaccccgtctcaaatcctacatgtga

SEQ ID NO: 103 - DNA, 882 bp, Prunus dulcis
  source 1..882: mol_type = genomic DNA
atggcgaaccaagtcgcggaccacctcgagccatggcgccacctccacggcaaggtcgtcatggtcaccggcgcttcgtctggcctcggccgcgagttctgcttggacttggctaaagctggatgcaggatcgtcgccgcggctcgccgcatcgaccgccttcaatccgtctgtgatgaaatcaaccagctaaccgttaccgcgccttcttcatcttcttcatcttcttctgtgaatgggcctggaagctccagggccgtggccgtggagctcgatatcagcgccgacgggccagctattgagaagtctgtaaacaaggcttgggacgcgtttgggcgcatcgatgccctcgttaacaacgccggtgttagaggtaatgtgagttcctcgctcgaattgtctgaggaggaatggaataatgtattcaagacaaatttgacagggtcctggttggtgtcaaaatatgtttctatacgcatgcgcgacgctgatcagggaggatcaatcatcaatgtatcttccattgcaggtcttcatcgtggatatttgcctggagctgtagcatacaattgttcaaaggctggtgtaaacaccttggcaaaggttatggctatggaattgggagtacacaaaatcagagtgaatgcaatatcgccaggacttttcagatctgaaatcacggagggtcttatgaaaaaagactggctacacaatgtggctatgaaaactgtccccttaagaacatttggcacttcggatccagcattgacatcactagttcgttatttagtacacgactcttctgaatatgtttcaggcaatgtctacattgttgatgcaggagccaccttgccaggtgtccctattttctctgccctttga

SEQ ID NO: 104 - DNA, 804 bp, Prunus dulcis
  source 1..804: mol_type = genomic DNA
atggggacttctcctgggaagaaagttttgctcacctccaatggggatgccatttcacacaatattgccttctctttggcccagcggggatgcaggctggtcttgatgggaaaggagagctgtctgcggagcattcaacagaaaataaagggctcattggagggtgtcgtgccagttgaggtggttgatgtagacatggaggacaagagagaaggagcttttgatgaggcagtggataaggcatgccacattttgggcaatttggatgcctttgtgcattgctatacttatgaagggaaaatgcaggagcatctagaattagctgaagatgagtttaaaaagataatgaagagaaactttatgtctgcatggtttctgttaaatgctgttggcagaagaatgcgagattataagtcaggaggttccatcatatttttgacctcgataattggagctgaaagagggatttatccaggagctgctgccaacagtgcatgctcggcagccgtgcagcagttagctaggacatcggctttggagattggtagataccagattagggtcaatgcaattgcccgtggtttgcacctagaagatgaatatccaatgtttgtgggtatggagagagcaaaaaagttggtgaaggaggcagctccattgcagagatggcttgatgttaaaaacgatctagcttcaactgtcatatatttaatcagtgatgggtcaaagtacatgacaggcacaaccatatttgttgatggggggcagtctttgacgagacctcggatgcgctcttatatgtga

SEQ ID NO: 105 - DNA, 864 bp, Solanum lycopersicum
  source 1..864: mol_type = genomic DNA
atggagacgcatgctaaatccgtgcgccagctggagccatggggagaactcgccggaaaagtggtgatggttaccggagcttcatcgggaatcggtagagatttctgctccgatttatcaaaagctggttgtagaatcatcgctgccgctcgtcgaatcgatcgattgcaatctctatgtgatgaaatcaactcgaattcatcgaacggatcgacgaagtcgagtcaggatttacgtgccgtagcgattgagcttgacgttagcgctaatggttctgccattgaagccgccgtacagaaagcttgggatgcatttggacgtatcgacggtttggttaataacgccggctttcgaggcagtgtgctttctccactggagttgtcggaggaggaatgggagaagatccacaaaacgaacttaagaggggcatggttggtgaccaaatatgtttgtatgcatatgcgcgctgctaatcaaggaggatccataatcaatatctcttctatcgctggtattaatcgtggacaactaccaggtagtcttgcttatacgtcttcaaaggaagctctcaacagcattacaaaggtgttggccctcgaattgggaccatacaagatcagagtgaactcaatatcacctggacttttcaaatctgagataacagagggtctcatacaaaaagactggattaaaaacattgaactgagaactattccgttgagaacacatggaacatcacatcccgctttaacttcagttgtacgttacctgatccacgattcctcggaatatgtttcaggtaacatgttcatagtagatgcaggagctactttacccggtgtcccgattttctcatccctctag

SEQ ID NO: 106 - DNA, 801 bp, Solanum lycopersicum
  source 1..801: mol_type = genomic DNA
atggaaaatcctggaaagaaggtgttgcttacctccaacggtgatgaaatttgcaacaacattgcgtaccatttagctcagaggggttgccaattggttttgatgggaaatgagcgccaattgaagagtgtagcggagaatataaagcaatcactaaagggtagtgttgccgtagaagttgtgggattggatatgacggaggatagagaaactgcttttgatgaagctgtggacaaggcatggaagatatttgggaagttggatgccttggtacactgctatgcttatgaagggaaaatgcaagatccactgcagctaattgatgacgagttcaaaaaaattgtcaaaataaattttatggctggatggtacctgttgaaatgtatcggtaacagaatgcgagacggtaaatctggaggatccattgtatttatgacctcgatcattggtgctgagagaggaatatatcaaggagctgctgcttacggttcatgtgccgctggaattcagcagctggttaggttatctgcaattgaattggggaagtaccaaatcagggtgaatggcatattgcgtggcttgcacctcgaggatgagtttcctttgtcagtgggtaaggagagagcagtgaagttgaccaaggaagcagcgcctctaaatcgatggcttgatcctaaaaaggatctggcttccactgtcatctatttaatcagtgatgattcaagatacatgacaggaacctccatctttgttgatggtgcccagtctctagtgaggccgcgaatgcgctcatatatgtga

(19) United States
(12) Patent Application Publication
(10) Pub. No.: US 2025/0034601 A1
(43) Pub. Date: Jan. 30, 2025

(54) HETERODIMERIC BENZALDEHYDE SYNTHASE, METHODS OF PRODUCING, AND USES THEREOF

(71) Applicant: Purdue Research Foundation, West Lafayette, IN (US)

(72) Inventors: Natalia Doudareva, Lafayette, IN (US); Xing-Qi Huang, West Lafayette, IN (US)

(73) Assignee: Purdue Research Foundation, West Lafayette, IN (US)

(21) Appl. No.: 18/704,380

(22) PCT Filed: Oct. 25, 2022
(86) PCT No.: PCT/US2022/078658

§ 371 Date: Apr. 24, 2024

Related U.S. Application Data
(60) Provisional application No. 63/271,280, filed on Oct. 25, 2021.

(51) Int. Cl. C12P 7/22; C12N 9/10; C12N 15/82

(52) U.S. Cl. CPC C12P 7/22; C12N 9/1029; C12N 15/8205; C12N 15/8243

(57) ABSTRACT

The disclosure generally relates to production of natural or semi-natural benzaldehyde and its derivatives using a heterodimeric benzaldehyde synthase comprising a benzaldehyde synthase alpha (BS) subunit and a benzaldehyde synthase beta (BS) subunit, nucleic acids encoding the subunits, an engineered heterodimeric benzaldehyde synthase, transgenic plants that produce the heterodimeric benzaldehyde synthases hereof, and resulting products containing natural or seminatural benzaldehyde.

### PRIORITY

The present application is related to and claims the priority benefit of U.S. Provisional Patent Application No. 63/271,280 filed Oct. 25, 2021, the content of which is hereby expressly incorporated by reference in its entirety into this disclosure.

### GOVERNMENT RIGHTS

This invention was made with government support under IOS-1655438 awarded by the National Science Foundation and Hatch project 177845 awarded by the United States Department of Agriculture. The government has certain rights in the invention.

### TECHNICAL FIELD

The disclosure generally relates to production of natural or semi-natural benzaldehyde and its derivatives using a heterodimeric benzaldehyde synthase comprising an alpha and a beta units, an engineered heterodimeric benzaldehyde synthase, transgenic plants that produce the heterodimeric benzaldehyde synthases hereof, and resulting products containing natural or semi-natural benzaldehyde.

### BACKGROUND

This section introduces aspects that may help facilitate a better understanding of the disclosure. Accordingly, these statements are to be read in this light and are not to be understood as admissions about what is or is not prior art.

Benzaldehyde (C₆H₅CHO) is the simplest aromatic aldehyde found in nature, consisting of a single benzene ring bearing an aldehyde group. Phylogenetically, it is one of the most widely distributed volatiles and is likely the most ancient compound given that it is produced by over 50% of plant families analyzed to date for their volatile profiles, but also by insects and non-insect arthropods. Knudsen et al., Diversity and distribution of floral scent., Bot. Rev. 72: 1-120 (2006); and Schiestl, The evolution of floral scent and insect chemical communication, Ecol. Lett. 13: 643-656 (2010).

Benzaldehyde can play important roles in chemical communications serving as sex, aggregation, and alarm pheromones. In addition, benzaldehyde can be a defense compound in some insects and non-insect arthropods, as well as a pollinator attractant, flavor volatile, and antifungal compound in plants. Schiestl (2010), supra. Found in scents of numerous flowers, benzaldehyde can be readily detected by hawk moths, eliciting strong responses from their antennas. Raguso and Light, Electroantennogram responses of male Sphinx perelegans hawkmoths to floral and ‘green-leaf volatiles’, Entomol. Experimentalis et Applicata 86: 287-293 (1998); and Hoballah et al., The composition and timing of flower odour emission by wild Petunia axillaris coincide with the antennal perception and nocturnal activity of the pollinator Manduca sexta, Planta 222: 141-150 (2005). Moreover, the loss of its emission can lead to a shift in reproductive strategy in the genus Capsella from pollination by insects to self-fertilization. Sas et al., Repeated inactivation of the first committed enzyme underlies the loss of benzaldehyde emission after the selfing transition in Capsella, Current Biology 26: 3313-3319 (2016).

Benzaldehyde possesses a characteristic pleasant almond-like odor and can contribute to aromas of many fruits including, for example, cherry, peach, cranberry, raspberry, and melon. Mayobre et al., Genetic dissection of aroma biosynthesis in melon and its relationship with climacteric ripening, Food Chem 353 (2021); and Verma et al., Natural benzaldehyde from Prunus persica (L.) Batsch, Int. J Food Properties 20: 1259-1263 (2017). Additionally, when emitted by postharvest tomato fruits, it can also inhibit Botrytis cinerea infection, thus preventing gray mold disease, which is a cause of significant economic loss in tomato fruit industries worldwide. Lin et al., Ethylene and benzaldehyde emitted from postharvest tomatoes inhibit Botrytis cinerea via binding to G-protein coupled receptors and transmitting with cAMP-signal pathway of the fungus, J Agr Food Chem 67: 13706-13717 (2019). Furthermore, benzaldehyde is present in almonds, apricots, apples, and cherry kernels as a diglucoside (i.e., amygdalin) from which it can be released by hydrolysis along with the toxic byproduct hydrogen cyanide. Sánchez-Pérez et al., Bitterness in almonds, Plant Physiol 146: 1040-1052 (2008). Long known for its smell and taste, benzaldehyde is the most important, after vanillin, contributor to the flavor industry. Verma et al. (2017), supra. It is of economic value to the cosmetic and fragrance industries and is used extensively as a precursor to plastic additives and some dyes.

To date, benzaldehyde is primarily produced synthetically by air oxidation of toluene. This existing preparation method has the defects of lower yield and can result in environmental pollution. Natural benzaldehyde, which constitutes approximately 1.5% of total annual world production, is mainly obtained by a retro-aldol reaction of natural cinnamaldehyde extracted from cassia oil. Brenna and Parmeggiani, Biotechnological production of flavors. In: Industrial Biotechnology: Products and Processes: 271-308, Wiley-VCH Verlag GmbH & Co. KGaA (Eds. Wittmann, C. & James C. Liao, J. C.) (2017).

Despite the natural benzaldehyde market growing each year, the method through which this simple aromatic compound is synthesized in plants remains unknown. Elucidation of the biosynthetic pathway of benzaldehyde can have a tremendous implication for many industries, including cosmetics, human foods, animal feeds, and flavors, as well as the pharmaceutical industry.

### Sequence Listings

The sequences herein (SEQ ID NOS: 1-106) are also provided in computer-readable form encoded in a file filed herewith and incorporated herein by reference. The information recorded in computer-readable form is identical to the written Sequence Listing provided below, pursuant to 37 C.F.R. § 1.821(f).

SEQ ID NO: 1 is an alpha subunit of the benzaldehyde synthase from Petunia hybrida (>PhBSα).

SEQ ID NO: 2 is a beta subunit of the benzaldehyde synthase from Petunia hybrida (>PhBSβ).

SEQ ID NO: 3 is the nucleotide sequence of the coding sequence (CDS) that encodes Petunia hybrida benzaldehyde synthase alpha subunit (>PhBSα).

SEQ ID NO: 4 is the nucleotide sequence of the CDS that encodes Petunia hybrida benzaldehyde synthase beta subunit (>PhBSβ).

SEQ ID NO: 5 an alpha subunit of the benzaldehyde synthase from Arabidopsis thaliana.

SEQ ID NO: 6 is an alpha subunit of the benzaldehyde synthase from Prunus dulcis.

SEQ ID NO: 7 is an alpha subunit of the benzaldehyde synthase from Solanum lycopersicum.

SEQ ID NO: 8 is a beta subunit of the benzaldehyde synthase from Arabidopsis thaliana.

SEQ ID NO: 9 is a beta subunit of the benzaldehyde synthase from Prunus dulcis.

SEQ ID NO: 10 is a beta subunit of the benzaldehyde synthase from Solanum lycopersicum.

SEQ ID NO: 11 is a 300 nucleic acid base pair fragment (CDS nucleotides 580˜879) of Nicotiana benthamiana Phytoene Desaturase (PDS) having the GenBank deposit accession number DQ469932.1.

SEQ ID NO: 12 is the amino acid sequence for an alpha unit of benzaldehyde synthase from Peaxi162Scf00811g00011.1, a homolog of PhBSα.

SEQ ID NO: 13 is an amino acid sequence for a beta unit of benzaldehyde synthase from Peaxi162Scf00776g00122.1, a homolog of PhBSβ.

SEQ ID NOS: 14-91 are each nucleic acid sequences for a reverse or forward primer, as identified in Table 1 of FIG. 24.

SEQ ID NO: 92 is a nucleic acid sequence that encodes AtBSα (AT3G55290).

SEQ ID NO: 93 is a nucleic acid sequence that encodes AtBSβ (AT3G01980).

SEQ ID NO: 94 is a nucleic acid sequence that encodes a synthetic construct T-DNA insertion line of AtBSα: TATTGAAAGAAAGTCCTGATTGCTG.

SEQ ID NO: 95 is a nucleic acid sequence that encodes a synthetic construct T-DNA insertion line of AtBSβ: TCAATAAATGATGAAGTTTTTTCTC.

SEQ ID NO: 96 is a nucleic acid sequence that encodes a synthetic construct T-DNA insertion line of AtBSβ: TCCCGTAAAATATCTTTTACTGCAT.

SEQ ID NO: 97 is an amino acid sequence for an alpha unit of benzaldehyde synthase from Prunus dulcis.

SEQ ID NO: 98 is an amino acid sequence for an alpha unit of benzaldehyde synthase from Prunus dulcis.

SEQ ID NO: 99 is an amino acid sequence for an alpha unit of benzaldehyde synthase from Solanum lycopersicum.

SEQ ID NO: 100 is an amino acid sequence for a beta unit of benzaldehyde synthase from Solanum lycopersicum.

SEQ ID NO: 101 is a nucleotide sequence of a CDS that encodes Arabidopsis thaliana benzaldehyde synthase alpha subunit mRNA.

SEQ ID NO: 102 is a nucleotide sequence of a CDS that encodes Arabidopsis thaliana benzaldehyde synthase beta subunit mRNA.

SEQ ID NO: 103 is a nucleotide sequence of a CDS that encodes Prunus dulcis benzaldehyde synthase alpha subunit mRNA.

SEQ ID NO: 104 is a nucleotide sequence of a CDS that encodes Prunus dulcis benzaldehyde synthase beta subunit mRNA.

SEQ ID NO: 105 is a nucleotide sequence of a CDS that encodes Solanum lycopersicum benzaldehyde synthase alpha subunit mRNA.

SEQ ID NO: 106 is a nucleotide sequence of a CDS that encodes Solanum lycopersicum benzaldehyde synthase beta subunit mRNA.

### SUMMARY

Methods for producing benzaldehyde (e.g., natural or semi-natural benzaldehyde) are provided. Such a method can comprise providing a biosynthesis platform comprising: (a) a first nucleic acid sequence encoding a benzaldehyde synthase alpha (BSα) subunit, and (b) a second nucleic acid sequence encoding a benzaldehyde synthase beta (BSβ) subunit. The first and second nucleic acid sequences can be overexpressed in the biosynthesis platform (e.g., at a molar ratio of 1:1). The method can further comprise subjecting the biosynthesis platform to conditions such that benzaldehyde is produced. In certain embodiments, the method further comprises isolating the benzaldehyde from the biosynthesis platform.

The BSα and BSβ subunits together (e.g., combined or taken together) can form a heterodimeric enzyme.

In certain embodiments, the methods hereof can additionally comprise: transforming eukaryotic cells or microbes with a vector carrying the first nucleic acid sequence under conditions that allow for the overexpression of the BSα subunit; transforming eukaryotic cells or microbes with a vector carrying the second nucleic acid sequence under conditions that allow for the overexpression of the BSβ subunit; selecting transformants that overexpress both BSα and BSβ subunits; and growing the transformants to facilitate de novo production of benzaldehyde in the biosynthesis platform.

In certain embodiments, the biosynthesis platform can comprise one or more populations of microbes. There, the methods can further comprise transforming a first population of microbes with a vector carrying the first nucleic acid sequence under conditions that allow for the overexpression of the BSα subunit; transforming a second population of microbes with a vector carrying the second nucleic acid sequence under conditions that allow for the overexpression of the BSβ subunit; selecting transformants that overexpress the BSα subunit from the first population of microbes; selecting transformants that overexpress the BSβ subunit from the second population of microbes; and mixing the BSα subunits from the first population of microbes with the BSβ subunits from the second population of microbes to produce benzaldehyde.

The first nucleic acid sequence, the second nucleic acid sequence, or both the first and second nucleic acid sequences can be heterologous to the platform. In certain embodiments, the first nucleic acid sequence is heterologous to the biosynthesis platform. In certain embodiments, the second nucleic acid sequence is heterologous to the biosynthesis platform. In certain embodiments, both the first and second nucleic acid sequences are heterologous to the biosynthesis platform. Additionally or alternatively, in certain embodiments, the first and second nucleic acid sequences are nucleotide sequences from the same species or the first and second nucleic acid sequences (as compared to each other) are nucleotide sequences from different species.

The first nucleic acid sequence can comprise a nucleotide sequence of SEQ ID NO: 3, SEQ ID NO: 101, SEQ ID NO: 103, SEQ ID NO: 105, or a nucleotide sequence that has at least 50% identity to SEQ ID NO: 3, SEQ ID NO: 101, SEQ ID NO: 103, or SEQ ID NO: 105. The second nucleic acid sequence can comprise a nucleotide sequence of SEQ ID NO: 4, SEQ ID NO: 102, SEQ ID NO: 104, SEQ ID NO: 106, or a nucleotide sequence that has at least 50% identity to SEQ ID NO: 4, SEQ ID NO: 102, SEQ ID NO: 104, SEQ ID NO: 106. The first nucleic acid sequence can encode SEQ ID NO: 1, SEQ ID NO: 5, SEQ ID NO: 6, SEQ ID NO: 7 or a functional fragment or homolog of SEQ ID NO: 1, SEQ ID NO: 5, SEQ ID NO: 6, or SEQ ID NO: 7. The second nucleic acid sequence can encode SEQ ID NO: 2, SEQ ID NO: 8, SEQ ID NO: 9, SEQ ID NO: 10, or a functional fragment or homolog of SEQ ID NO: 2, SEQ ID NO: 8, SEQ ID NO: 9, or SEQ ID NO: 10.

In certain embodiments, the first nucleic acid sequence encodes SEQ ID NO: 1, SEQ ID NO: 5, SEQ ID NO: 6, SEQ ID NO: 7 or a functional fragment or homolog of SEQ ID NO: 1, SEQ ID NO: 5, SEQ ID NO: 6, or SEQ ID NO: 7; and the second nucleic acid sequence encodes SEQ ID NO: 2, SEQ ID NO: 8, SEQ ID NO: 9, SEQ ID NO: 10, or a functional fragment or homolog of SEQ ID NO: 2, SEQ ID NO: 8, SEQ ID NO: 9, or SEQ ID NO: 10.

The biosynthesis platform can comprise genetically engineered microbes or genetically engineered eukaryotic cells, tissues, organs, or organisms. In certain embodiments, the biosynthesis platform can comprise genetically engineered algae, insect, or animal cells. In certain embodiments, the biosynthesis platform comprises genetically engineered microbes of an Escherichia coli strain, a Saccharomyces cerevisiae strain, or a Pichia pastoris strain in a fermentation medium. Where the biosynthesis platform comprises genetically engineered microbes in a fermentation medium, isolating the benzaldehyde can comprise recovering the benzaldehyde from the fermentation medium after fermentation.

In certain embodiments, the biosynthesis platform comprises a transgenic plant, a transgenic plant cell, a transgenic plant tissue, and/or a transgenic plant organ. For example, and without limitation, the transgenic plant, transgenic plant cell, transgenic plant tissue, and/or transgenic plant organ is or is obtained from Petunia hybrida, Nicotiana benthamiana, or Prunus dulcis.

Where the biosynthesis platform comprises a transgenic plant, a transgenic plant cell, transgenic plant tissue and/or a transgenic plant organ, isolating the benzaldehyde from the biosynthesis platform can comprise isolating the benzaldehyde from the transgenic plant, transgenic plant cell transgenic plant tissue and/or transgenic plant organ after growth.

The methods can further comprise supplying benzoyl-CoA, nicotinamide adenine dinucleotide phosphate (NADPH), or both benzoyl-CoA and NADPH to the biosynthesis platform. In certain embodiments, the method comprises supplying benzoyl-CoA, NADPH, or both benzoyl-CoA and NADPH to the mixture (e.g., combination) of the BSα and BSβ subunits. In certain embodiments, the method comprises supplying the benzoyl-CoA, NADPH, or both benzoyl-CoA and NADPH to a fermentation medium comprising the BSα and BSβ subunits. In certain embodiments, the method comprises upregulating benzoyl-CoA and/or NADPH production in a transgenic plant.

The methods hereof can further comprise purifying the BSα and BSβ subunits.

In certain embodiments, the molar ratio of the BSα subunit to the BSβ subunit (e.g., in the biosynthetic platform or mixture) is about 1:1.

Active heterodimeric enzymes are also provided. In certain embodiments, the active heterodimeric enzymes comprise an enzyme prepared according to any of the methods described herein.

In certain embodiments, each of the first species and the second species of the active heterodimeric enzyme is independently within an Arabidopsis genus, a Petunia genus, a Prunus, genus or a Solanum genus. The first species and the second species can be the same species or different species. The active heterodimeric enzyme can have a higher benzaldehyde synthetase activity than a wild-type benzaldehyde synthetase activity of the first species or the second species.

In certain embodiments, the first species is Petunia hybrida or Solanum lycopersicum and the second species is not within the Arabidopsis genus. In certain embodiments of the enzyme, the first species is Arabidopsis thaliana and the second species is Prunus dulcis, Petunia hybrida, or Solanum lycopersicum. In certain embodiments of the enzyme, the first nucleic acid sequence encodes an Arabidopsis thaliana BSα subunit and the second nucleic acid sequence encodes a Prunus dulcis BSβ subunit. In certain embodiments of the enzyme, the first nucleic acid sequence encodes an Arabidopsis thaliana BSα subunit and the second nucleic acid sequence encodes a Petunia hybrida BSβ subunit. Still further, in certain embodiments, the first nucleic acid sequence of the active heterodimeric enzyme encodes a Solanum lycopersicum BSα subunit and the second nucleic acid sequence encodes a Solanum lycopersicum BSβ subunit.

The enzyme can be specific towards benzoyl-CoA in the biosynthesis of natural or semi-natural benzaldehyde. NADPH can be a cofactor of the enzyme.

The BSα subunit of the enzyme can be or comprise a nucleotide sequence of SEQ ID NO: 3 or a nucleotide sequence having at least 50% identity to SEQ ID NO: 3 (e.g., that encodes a BSα subunit). The BSα subunit can be or comprise SEQ ID NO: 101, SEQ ID NO: 103, or SEQ ID NO: 105.

The BSβ subunit of the enzyme can be or comprise a nucleotide sequence of SEQ ID NO: 4 or a nucleotide sequence having at least 50% identity to SEQ ID NO: 4 (e.g., that encodes a BSβ subunit). The BSβ subunit can be or comprise SEQ ID NO: 102, SEQ ID NO: 104, or SEQ ID NO: 106.

Transgenic plants are also provided. The transgenic plants hereof can produce benzaldehyde at a rate that is at least 4-fold greater than benzaldehyde production in a corresponding wild-type plant. In certain embodiments, the benzaldehyde production of the transgenic plant is at or near 7.8-fold greater than benzaldehyde production in a corresponding wild-type plant.

In certain embodiments, such transgenic plants comprise a first heterologous nucleic acid sequence encoding a BSα subunit and a second heterologous nucleic acid sequence encoding a BSβ subunit, wherein one or both of the first and second nucleic acid sequences is/are operably linked to a regulatory element for directing expression of the first and/or second nucleic acid sequences. Additionally, (i) the transgenic plant can overexpress at least the BSβ subunit, (ii) the first heterologous nucleic acid can be from a first species and the second heterologous nucleic acid can be from a second species, or (iii) both (i) and (ii). In certain embodiments, both the first species and the second species are derived from a NAD(P)-binding Rossmann-fold superfamily.

In certain embodiments, the transgenic plant overexpresses both the BSα subunit and the BSβ subunit.

The first nucleic acid sequence can be or comprise a nucleotide sequence of SEQ ID NO: 3, or a nucleotide sequence that is at least 50% identity to SEQ ID NO: 3 and encodes a BSα subunit. In certain embodiments, the first nucleic acid sequence is or comprises SEQ ID NO: 101, SEQ ID NO: 103, or SEQ ID NO: 105. For example and without limitation, the first nucleic acid sequence can encode SEQ ID NO: 1, SEQ ID NO: 5, SEQ ID NO: 6, SEQ ID NO: 7 or a functional fragment or homolog of SEQ ID NO: 1, SEQ ID NO: 5, SEQ ID NO: 6, or SEQ ID NO: 7.

The second nucleic acid sequence can be or comprise a nucleotide sequence of SEQ ID NO: 4, or a nucleotide sequence that is at least 50% identity to SEQ ID NO: 4 and encodes a BSβ subunit. In certain embodiments, the second nucleic acid sequence is or comprises SEQ ID NO: 102, SEQ ID NO: 104, or SEQ ID NO: 106. For example and without limitation, the second nucleic acid sequence can encode SEQ ID NO: 2, SEQ ID NO: 8, SEQ ID NO: 9, SEQ ID NO: 10, or a functional fragment or homolog of SEQ ID NO: 2, SEQ ID NO: 8, SEQ ID NO: 9, or SEQ ID NO: 10.

In certain embodiments of the transgenic plant, the first nucleic acid sequence encodes SEQ ID NO: 1, SEQ ID NO: 5, SEQ ID NO: 6, SEQ ID NO: 7 or a functional fragment or homolog of SEQ ID NO: 1, SEQ ID NO: 5, SEQ ID NO: 6, or SEQ ID NO: 7; and the second nucleic acid sequence encodes SEQ ID NO: 2, SEQ ID NO: 8, SEQ ID NO: 9, SEQ ID NO: 10, or a functional fragment or homolog of SEQ ID NO: 2, SEQ ID NO: 8, SEQ ID NO: 9, or SEQ ID NO: 10.

In certain embodiments, the transgenic plant is Petunia hybrida, Nicotiana benthamiana, or Prunus dulcis.

The first nucleic acid sequence of the transgenic plant can be from a first species and the second nucleic acid sequence can be from a second species. The first species and the second species can be independently within an Arabidopsis genus, a Petunia genus, a Prunus genus, or a Solanum genus. The first species and the second species can be the same or different species. The first species and/or the second species can be the same species as the transgenic plant or different species from the transgenic plant. In certain embodiments, the first species is Arabidopsis thaliana and the second species is Prunus dulcis, Petunia hybrida, or Solanum lycopersicum.

The regulatory element of the transgenic plant can comprise, for example, a tissue-specific promoter for directing expression of the first and/or second nucleic acid sequence in the cells of a leaf, a root, a flower, a developing ovule or a seed of the transgenic plant.

### BRIEF DESCRIPTION OF THE DRAWINGS

The disclosed embodiments and other features, advantages, and aspects contained herein, and the matter of attaining them, will become apparent in light of the following detailed description of various exemplary embodiments of the present disclosure. Such detailed description will be better understood when taken in conjunction with the accompanying drawings. Identical reference numerals have been used, where possible, to designate identical features that are common to the figures.

FIG. 1 shows a schematic representation of the initial proposed pathways for benzaldehyde biosynthesis in plants, with enzymes responsible for each biochemical reaction shown in bold black, established biochemical reactions represented by solid black arrows, and conventionally unidentified steps shown by dashed arrows. Transporters involved in metabolite transport between different subcellular locations are circled with a dashed line, with the flow of metabolites indicated with “X” on the arrows. Abbreviations: CHD, cinnamoyl-CoA hydratase/dehydrogenase; CNL, cinnamate-CoA ligase; CTS, peroxisomal cinnamic acid/cinnamoyl-CoA transporter COMATOSE. The substrate specificity of CTS is unclear, as indicated by a cyan question mark; KAT, 3-ketoacyl thiolase; PAL, phenylalanine ammonia lyase; pCAT, plastidial cationic amino acid transporter; Phe, phenylalanine; 3O3PP-CoA, 3-oxo-3-phenylpropanoyl-CoA.

FIG. 2A shows two predicted routes for the biosynthesis of benzaldehyde and its predicted labeling from [²H₈]-Phe, with established biochemical reactions represented by solid arrows and conventionally unidentified steps shown with dashed arrows; PAL, phenylalanine ammonia lyase.

FIGS. 2B-2D show gas chromatography-mass spectrometry (GC-MS) chromatograms of benzaldehyde (FIG. 2B), benzylalcohol (FIG. 2C), and benzylbenzoate (FIG. 2D) produced in vivo by petunia petals fed with [²²H₈]-Phe for 2 hours presented as extracted ion currents of indicated m/z.

FIGS. 2E-2G shows unlabeled compounds (lines labeled with B), overlayed with the newly synthesized compounds from [²²H₈]-Phe labeled (lines labeled with A) mass spectra for benzaldehyde (FIG. 2E), benzylalcohol (FIG. 2F) and benzylbenzoate (FIG. 2G) of FIGS. 2B-2D, with the response of the most abundant peak in each graph set to 100%;

FIGS. 3A-3C show data related to the partial purification of native Petunia hybrida benzaldehyde synthase (PhBS) from petunia flowers, with FIG. 3A showing GC-MS analyses of benzaldehyde synthase (BS) activity in petunia flower crude protein extracts+benzoyl-CoA+nicotinamide adenine dinucleotide phosphate (NADPH) (a); crude extracts+benzoyl-CoA+NADP⁺ (b); crude extracts+benzoyl-CoA+nicotinamide adenine dinucleotide (NADH) (c); crude extracts+benzoyl-CoA+NAD⁺ (d); crude extracts+benzoyl-CoA (e); crude extracts+benzoic acid+NADPH (f); heat denatured crude extracts+benzoyl-CoA+NADPH (g); and crude extracts+cinnamic acid (h), with the compounds labeled with numbers being: (1) benzaldehyde, (2) benzylalcohol, (3) internal standard (naphthalene), and (4) benzylbenzoate, and the response of internal standard in each run set as 100%; FIG. 3B showing a DE53 anion exchange chromatography of BS activity in petunia crude extracts (fractions with BS activity are numbered); FIG. 3C showing MonoQ™ anion exchange chromatography of combined BS activity containing fractions 14 and 15 from DE53 chromatography of FIG. 3B (fractions with profound BS activity are numbered); and FIG. 3D showing a sodium dodecyl-sulfate polyacrylamide gel electrophoresis (SDS-PAGE) analysis of purification steps for PhBS (active fractions from successive purification steps were run on 12% SDS-PAGE); the indicated lanes correspond to: Crude, petunia flower crude protein extract (˜40 μg); 50-60%, proteins precipitated at 50˜60% ammonium sulphate saturation (˜40 μg); DE53, combined fractions 14 and 15 after DE53 chromatography (˜20 μg); and 22˜27, fractions separated by MonoQ™ chromatography shown in (c) of FIG. 3A (10 μL each); asterisk (*) indicating the size of native PhBS (around 60 kDa); triangle indicating the position of two closely migrated bands representing PhBSα and PhBSβ; total BS activities (pKat) and intensity density data of both bands listed below each gel for fractions 23 to 26.

FIGS. 4A-4C show data related to the heterodimeric nature and substrate specificity of PhBS, with FIG. 4A shows GC-MS chromatograms from products formed by PhBSα and PhBSβ subunits, and their mixture at 1:1 ratio (the response of internal standard in each run was set as 100%); FIG. 4B shows GC-MS chromatograms of products formed by PhBS using various different hydroxycinnamoyl-CoA substrates; FIG. 4C shows data from a pull-down analysis of PhBSβ-His binding to MBP-PhBSα, where purified MBP-PhBSα was incubated with bacterial lysate of pET32b empty vector (EV) or pET32b expressing PhBSβ-His, and protein complex was purified using Amylose resin and analyzed by SDS-PAGE (* indicating the position of MBP-PhBSα, and triangle indicating the position of PhBSβ-His); FIG. 4D shows data from a pull-down analysis of PhBSα binding to PhBSβ-His (* indicating the position of MBP-PhBSα; ** indicating the position of free MBP tag; triangle representing the position of PhBSβ-His and untagged PhBSα, as indicated by BS activity); FIG. 4E shows yeast two-hybrid screen (Y2H) detection of PhBSα and PhBSβ interactions, where yeast cells harboring different combinations of activation domain of pGAD-T7 (AD) and DNA binding domain of pGBK-T7 (BD) were spotted at increasing dilutions on nonselective (-leu/-trp) and selective medium (-leu/-trp/-his) (EV, empty vector) (AD, activation domain of pGAD-T7 and BD, to which petunia BSα and BSβ were fused; EV, empty vector).

FIGS. 5A-5I show data related to studies on the function of PhBS in vivo. FIG. 5A shows graphical data on the changes in PhBSα (black bars) and PhBSβ6 (white bars) transcript levels during a normal light/dark cycle in petunia corolla harvested from day 1 post-anthesis (15:00) to day 3 post-anthesis (3:00), where PhBSα and PhBSβ6 expression was determined by quantitative real-time polymerase chain reaction (qRT-PCR) with gene-specific primers and expressed as a copy number of transcripts per microgram of total RNA×10⁶ (white and gray areas correspond to light and dark, respectively; box and whiskers plot show (n=4 biological replicates), center line, median; box limits, upper and lower quartiles; whiskers, minimum and maximum. FIGS. 5B-5D are related to the effect of PhBS downregulation on benzaldehyde emission and, more specifically, FIG. 5B shows transcript levels of PhBSα and PhBSβ6 in pds control (black bars) and pds-bsα-bsβ (white bars) in 2-day-old VIGS flowers at 21:00 hours determined by qRT-PCR and presented relative to the corresponding levels in pds control set as 1 (data are means±SE (n=6 biological replicates); P values determined by two-way analysis of variance (ANOVA) multiple comparisons test relative to the pds controls); FIG. 5C shows data related to BS activities in crude extracts prepared from corollas of 2-day-old VIGS flowers harvested at 21:00 hours (data are means SE (n=6 biological replicates)); and FIG. 5D shows data related to benzaldehyde emission in 2-day-old VIGS flowers (volatiles were collected from 20:00 hours until 21:00 hours (data are means±SE (n=6 biological replicates)). P values shown in FIGS. 5C and 5D were determined by unpaired two-tailed Student's t-test relative to pds control). FIGS. 5E-5i are related to the reconstitution of benzaldehyde biosynthetic pathway in N. benthamiana leaves, with FIG. 5E showing a biosynthetic pathway for benzaldehyde in petunia flowers, with enzymes used for pathway reconstitution shown in bold (the enzyme responsible for benzaldehyde reduction to benzylalcohol in petunia is unknown as indicated by the question mark); FIG. 5F showing a GC-MS analysis of infiltrated N. benthamiana leaves after mock feeding; FIG. 5G showing a GC-MS analysis of infiltrated tobacco leaves after Phe feeding for 24 hours; and FIG. 5H showing a GC-MS analysis of infiltrated tobacco leaves after Phe feeding and Viscozyme treatment. The constructs used for Agrobacterium infiltrations are shown in FIGS. 5F-5H on the right, and the response of internal standard in each run was set as 100%. FIG. 5I shows data related to the quantification of benzylalcohol formation in infiltrated leaves after Viscozyme treatment shown in FIG. 5H (data are means±SE (n=5 biological replicates); P values determined by ordinary one-way ANOVA multiple comparisons test relative to the EV control).

FIGS. 6A and 6B relate to cross-species interactions of BS subunits, with FIG. 6A showing a protein sequence identity matrix between BS subunits from petunia (PhBS), Arabidopsis (AtBS), almond (PdBS) and tomato (SlBS) with the cells boxed in heavy black lines having the highest sequence identity (e.g., greater than about 50% sequence identity, greater than about 60% sequence identity, and greater than about 70% sequence identity); and FIG. 6B showing apparent activities of cross-species BS heterodimers (freshly purified recombinant proteins were mixed at 1:1 ratio and incubated on ice overnight before standard BS activity assays; data are means±SE (n=3 technical replicates); cells with significant fold changes related to the activity of an α subunit with a β subunit relative to its original homologous BS activity are highlighted with a heavy black box (e.g., greater than about 2-fold change, greater than about 3-fold change, greater than about 4-fold change, greater than about 5-fold change, and greater than about 6-fold change).

FIG. 7 shows graphical data related to benzaldehyde synthase (BS) activity and related protein size within a small fraction (200 μL) of crude petunia flower protein extract using a Superose 12 10/300 size exclusion column and plotted against elution volume (Activity line), where the Superose 12 column was calibrated with the following standard proteins: (1) β-amylase (200 kDa), (2) alcohol dehydrogenase (150 kDa), (3) bovine serum albumin (66 kDa), (4) carbonic anhydrase (29 kDa), and (5) cytochrome c (12.4 kDa).

FIG. 8 shows SDS-PAGE analysis of fractions from size exclusion chromatography (SEC), such fractions taken from the samples of FIG. 7 (identified as SEC 19 to SEC 24), with total BS activity (pKat·mg protein⁻¹) and intensity density of BS subunits for fractions SEC 22 to SEC 24 shown below the gel (M, protein molecular weight standards; triangle indicating the position of BS subunits).

FIG. 9 shows a phylogenetic analysis of benzaldehyde synthase homologs in the Petunia genus, where the tree is drawn to scale with branch lengths measured in the number of substitutions per site.

FIG. 10 shows a phylogenetic analysis of benzaldehyde synthase subunits in the Solanaceae family, where the tree is drawn to scale with branch lengths in the same units as those of the evolutionary distances used to infer the phylogenetic tree. The evolutionary distances were computed using the p-distance method and are shown in the units of the number of amino acid differences per site (129 amino acid sequences were analyzed (SEQ ID NOS: 17-138)).

FIGS. 11A and 11B show SDS-PAGE gels relating to the purification of benzaldehyde synthetase subunits and purified recombinant petunia 4-coumarate: CoA ligase (Ph4CL1) from E. coli, with FIG. 11A showing data regarding aliquots of fractions from prokaryotic expression and purification of maltose binding protein (MBP)-tagged PhBS subunits and FIG. 11B showing data regarding purification of Ph4CL1, where total soluble bacterial lysate after isopropyl-β-D-thiogalactopyranoside (IPTG) induction is labeled as “Crude”, and ˜ 2 μg of purified protein after elution with 10 mM maltose solution is labeled as “Purified”. The triangle indicates the position of MBP-tagged pHBS units in FIG. 11A and the position of Ph4CL1 protein in FIG. 11B; MBP is about 42.5 kDA in size.

FIG. 12A shows confocal laser scanning microscopy images of transient biomolecular fluorescence complementation (BiFC) analysis of the interaction of PhBSα subunit and the PhBSβ subunit in N. benthamiana leaves where the “FP” panels (yellow) represent signals of reconstituted enhanced yellow fluorescent protein (EYFP) as a result of protein-protein interactions; the “mCherry-PTS1” panels (magenta) represent the signal of the peroxisome-targeted mCherry marker protein; the “Merged” panels show merged EYFP and mCherry signals; and blue signals in the “Merged” panels represent chlorophyl autofluorescence. Further, schematic diagrams of the protein topologies and combinations for each experiment are shown on the right as follows (from amino terminus (N-terminus) to carboxyl terminus (C-terminus)): (1) nEYFP-PhBSα+cEYFP-PhBSβ; (2) nEYFP-PhBSα+cEYFP-PhBSβ captured with 63× oil immersion objective lens; (3) nEYFP+cEYFP; (4) nEYFP-PhBSα+cEYFP; and (5) nEYFP+cEYFP-PhBSβ (the experiment was repeated three times with similar results each time).

FIG. 12B shows confocal laser scanning microscopy images of the subcellular localization of PhBS subunits expressed in N. benthamiana leaves where the “FP” panels (yellow) represent signals of PhBS fused fluorescent proteins; the “mCherry-PTS” panels (magenta) represent signal of a peroxisome-targeted mCherry marker protein; the “Merged” panels show merged FP and mCherry signals; and the blue signals in the “Merged” panels represent chlorophyll autofluorescence. Schematic diagrams of the BS-FP fusion proteins for each experiment are illustrated on the right as follows (from amino terminus (N-terminus) to carboxyl terminus (C-terminus)): (6) GFP-PhBSα; (7) GFP-PhBSβ; (8) PhBSα-EYFP; (9) PhBSβ-EYFP; and (10) EYFP (FP, fluorescent protein; PTS, putative peroxisomal targeting signal). All images in FIGS. 12A and 12B were captured using 20× objective lens except for (2) in FIG. 12A. Scale bars=20 μm, except for (2) in FIG. 12A, in which bars are 5 μm.

FIG. 13 shows data from a GC-MS analysis of products formed by MBP-tagged PhBS from different short-chain fatty acyl-CoA substrates and relates to substrate specificity of purified PhBS. The response of internal standard in each run was set as 100%.

FIGS. 14A and 14B relate to the expression in planta of PhBSα and PhBSβ in petunia flowers, with FIG. 14A showing data on the tissue-specific expression of PhBSα (black bars) and PhBSβ (white bars) and FIG. 14B showing data on the developmental expression of PhBSα (black bars) and PhBSβ (white bars) in petunia corolla from mature buds to day 7 post-anthesis. The PhBSα and PhBSβ expression was determined by qRT-PCR with gene-specific primers and is expressed as copy number of transcripts per microgram of total RNA. The data shown are means±SE (n=4 biological replicates for FIG. 14A, and n=3 biological replicates for FIG. 14B).

FIG. 15 shows plot data related to the effect of PhBS downregulation on the emission of individual petunia volatiles, with emission rates of individual volatile organic compounds (VOCs) shown in pds (control) and pds-bsα-bsβ virus-induced gene silencing (VIGs) petunia flowers. Volatiles were collected from 2-day-old flowers from 20:00 until 21:00. Data are means±SE (n=6 biological replicates), and P values, shown on top of each graph, as determined by unpaired two-tailed Student's t-test relative to pds control.

FIG. 16 shows graphical data of transcript levels in pds-bsα-bsβ VIGS flowers measured at 9:00 P.M. and presented relative to the corresponding levels in pds control (set as 1) to assess the effect of PhBs downregulation on expression of scent biosynthetic genes in petunia flowers. Transcript levels were determined by qRT-PCR with gene-specific primers in pds (control) and pds-bsα-bsβ. Data are means±SE (n=6 biological replicates), and P values determined by two-way ANOVA multiple comparisons test relative to the pds controls. The displayed gene identifiers encode the following proteins: BPBT, benzoyl-CoA: benzyl alcohol/2-phenylethanol benzoyltransferase; BSMT, benzoic acid/salicylic acid carboxyl methyltransferase; EGS, eugenol synthase; IGS, isoeugenol synthase; PAAS, phenylacetaldehyde synthase; and PAL, phenylalanine ammonia lyase.

FIG. 17 shows a phylogenetic analysis of BSβ homologs in land plants, with the tree drawn to scale with branch lengths measured in the number of substitutions per site (63 amino acid sequences were analyzed).

FIGS. 18A and 18B show alignment of deduced amino acid sequences for the BS subunits from the four species P. hybrida (petunia), A. thaliana (Arabidopsis), Prunus dulcis (almond) and S. lycopersicum (tomato), with FIG. 18A showing the alignment of amino acid sequences for BSα subunits (P. hybrida (PtBSα) (SEQ ID NO: 1), A. thaliana (Arabidopsis (AtBSα) (SEQ ID NO: 5), Prunus dulcis (PdBSα) (SEQ ID NO: 6), and S. lycopersicum (SlBSα) (SEQ ID NO: 7)) and FIG. 18B showing the alignment of amino acid sequences for BSβ subunits ((P. hybrida (PtBSβ) (SEQ ID NO: 2), A. thaliana (Arabidopsis (AtBSβ) (SEQ ID NO: 8), Prunus dulcis (PdBSβ) (SEQ ID NO: 9), and S. lycopersicum (SlBSβ) (SEQ ID NO: 10)). Conserved residues are boxed in a heavy black line, while similar residues are boxed in a light black line.

FIG. 19A-19E relate to studies on the purification and characterization of BS from phylogenetically distant species, with FIG. 19A showing the results of an SDS-PAGE analysis of about 2 μg of purified MBP-tagged BS subunits from petunia, Arabidopsis, almond and tomato (M, protein molecular weight standards); FIGS. 19B-19D showing the results of a GC-MS analysis of products formed in vitro by Arabidopsis (FIG. 19B), almond (FIG. 19C) and tomato (FIG. 19D) purified recombinant BS proteins in enzymatic assays, with the response of internal standard in each run set as 100%; FIG. 19E showing the results of gel filtration chromatography of purified recombinant PhBS subunits, AtBSβ and PhBSα-AtBSβ hybrid (peak labels are as follows: *, dimers; **, tetramers; and ***, multimers; STD, chromatogram of calibration standards).

FIGS. 20A-20E relate to the expression profile of AtBSα and AtBSβ. Tissue-specific and developmental expression levels of AtBSα (FIG. 20A) and AtBSβ(FIG. 20B) transcripts are shown, with the data in FIG. 20A and FIG. 20B obtained from the publicly available ePlant online directory. FIGS. 20C and 20D show results of peptide enrichment of AtBSα (FIG. 20C) and AtBSβ (FIG. 20D) in different tissues with the data in FIG. 20C and FIG. 20D retrieved from the publicly available Arabidopsis PeptideAtlas project website. Column names represent distinct observed peptides of each protein, and an asterisk denotes single genome mapping. FIG. 20E shows a hierarchical clustering of AtBS genes as well as genes involved in β-oxidation and lignin biosynthesis pathways; the plot was generated using the publicly available ATTED-II Hcluster tool. Genes used to build the plot of FIG. 20E are as follows: AtBSα (AT3G55290; SEQ ID NO: 92), AtBS/I (AT3G01980; SEQ ID NO: 93), PAL1 (AT2G37040), PAL2 (AT3G53260), PAL3 (AT5G04230), PAL4 (AT3G10340), 4CL (AT4G19010), CCR1 (AT1G15950), CCR2 (AT1G80820), C4H (AT2G30490), HCT (AT5G48930), CAD1 (AT1G72680), CCoAOMT1 (AT4G34050), F5H (AT4G36220), Peroxidase (AT5G66390), PRX52 (AT5G05340), CCOAMT (AT1G67980), KAT1 (AT1G04710), KAT2 (AT2G33150), AIM1 (AT4G29010), MFP2 (AT3G06860), and AAE12 (AT1G65890). The subcluster containing both AtBSα and AtBSβ is highlighted with a box.

FIGS. 21A-21F show data related to the characterization of Arabidopsis bs mutants. FIG. 21A are schematic diagrams showing the gene structures of AtBSα (SEQ ID NO: 92), AtBSβ (SEQ ID NO: 93), and T-DNA insertion sites in five bs mutant lines. FIG. 21B shows genotyping data of the bs mutants prepared using polymerase chain reaction (PCR) to analyze the genomic regions flanking each T-DNA insertion and a corresponding region of the wild-type gene. FIG. 21C shows data resulting from a qRT-PCR analysis of AtBSα and AtBS/I transcripts in floral tissues of Arabidopsis wild type (ecotype Col-0) and bs mutants using gene-specific primers (expression of each gene in wild type was set to 1; data are means±SE (n=3 biological replicates, each replicate contains >100 fully opened flowers and flower buds); P values determined by two-way ANOVA multiple comparisons test of each gene relative to the Col-0 controls). FIG. 21D shows the results of a GC-MS analysis of BS activities in crude flower extracts from Arabidopsis wild type and bs mutants (response of internal standard in each run set to 100%). FIG. 21E shows the quantification of the apparent BS activities shown in FIG. 21D (BS activity in wild type set to 1; data are means SE (n=3 biological replicates). FIG. 21F shows the quantification of benzylalcohol formation in in vitro assays with the crude extracts shown in FIG. 21D (benzylalcohol level produced by a crude extract from wild type was set to 1; data are means±SE (n=3 biological replicates); P values in FIGS. 21E and 21F were determined by ordinary one-way ANOVA multiple comparisons test relative to the wild type controls).

FIG. 22 shows data related to substrate specificity of purified MBP-tagged AtBS; more specifically, the results of a GC-MS analysis of products formed by AtBS from different hydroxycinnamoyl-CoA substrates with the formation of cinnamaldehyde and coniferaldehyde by purified PhCCR1 used as a positive control. The combined EICs of mass units 106 (benzaldehyde), 128 (internal standard), 131 (cinnamaldehyde), and 178 (coniferaldehyde) are shown in FIG. 22 and the response of internal standard in each run was set as 100%.

FIG. 23 shows a schematic representation of a VOC biosynthetic network in petunia flowers with the enzymes responsible for each biochemical reaction shown in bold black text, established biochemical reactions presented by solid black arrows, unidentified steps shown by dashed arrows, transporters involved in metabolite transport between different subcellular locations circled by dashed-line circles, and the flow of metabolites indicated by arrows with an X positioned thereon. Cross-organelle transport of metabolites by unknown transporters or diffusion are indicated with question marks. Abbreviations: BALDH, benzaldehyde dehydrogenase; BPBT, benzoyl-CoA:benzyl alcohol/2-phenylethanol benzoyltransferase; BS, benzaldehyde synthase; BSMT, benzoic acid/salicylic acid carboxyl methyltransferase; CAD, cinnamyl alcohol dehydrogenase; CCR, cinnamoyl-CoA reductase; CFAT, coniferyl alcohol acyltransferase; CHD, cinnamoyl-CoA hydratase/dehydrogenase; CNL, cinnamate-CoA ligase; CTS, peroxisomal cinnamic acid/cinnamoyl-CoA transporter COMATOSE; EGS, eugenol synthase; IGS, isoeugenol synthase; KAT, 3-ketoacyl thiolase; PAAS, phenylacetaldehyde synthase; PAL, phenylalanine ammonia lyase; pCAT, plastidial cationic amino acid transporter; Phe, phenylalanine; 3H3PP-CoA, 3-hydroxy-3-phenylpropanoyl-CoA; 303PP-CoA, 3-oxo-3-phenylpropanoyl-CoA; TE, thioesterase; VS, vanillin synthase.

FIG. 24 shows Table 1, which is a list of primers used in the examples described herein.

### DETAILED DESCRIPTION

For the purposes of promoting an understanding of the principles of the present disclosure, reference will now be made to the embodiments illustrated in the drawings and specific language will be used to describe the same. It will nevertheless be understood that no limitation of scope is intended by the description of these embodiments. On the contrary, this disclosure is intended to cover alternatives, modifications, and equivalents as may be included within the spirit and scope of this application as defined by the appended claims.

As previously noted, while this technology can be illustrated and described in one or more preferred embodiments, the nanoparticles, compositions, and methods hereof can comprise many different configurations, forms, materials, and accessories. Further, in the following description, numerous specific details are set forth to provide a thorough understanding of the present disclosure. Particular examples can be implemented without some or all of these specific details, and it is to be understood that this disclosure is not limited to particular biological systems, which can, of course, vary.

To elucidate benzaldehyde biosynthesis in planta, Petunia hybrida cv. Mitchell flowers (which produce high levels of benzaldehyde) were used as a model system. Boatright et al., Understanding in vivo benzenoid metabolism in petunia petal tissue, Plant Physiology 135: 1993-2011 (2004); and Verdonk et al., Regulation of floral scent production in petunia revealed by targeted metabolomics, Phytochemistry 62: 997-1008 (2003). By combining in vivo stable isotope labeling with classical biochemical, proteomics and genetic approaches, it was determined that benzaldehyde is synthesized via the β-oxidative pathway, and a heterodimeric enzyme consisting of alpha (α) and beta (β) subunits, both of which belong to the NAD(P)-binding Rossmann-fold superfamily, is responsible for its formation. Alone, such subunits are catalytically inactive, but when mixed (e.g., in equal amounts), the subunits form an active enzyme. It has further been established that this active enzyme exhibits strict substrate specificity towards benzoyl-CoA and, in certain instances, uses NADPH as a cofactor.

In view of these significant findings, novel enzymes, transgenic plants, and methods for producing benzaldehyde and its derivatives are provided. The transgenic plants hereof can produce naturally-derived benzaldehyde and its derivatives in amounts significantly greater than those produced by corresponding wild-type plants and other biosynthesis platforms (e.g., over 6-fold greater, over 5-fold greater, over 4-fold greater, over 3-fold greater, or over 2-fold greater). As used herein, “naturally-derived” in the context of a product (e.g., benzaldehyde or its derivatives produced according to the methods provided herein) means a substance produced without the use of synthetic chemicals, but instead by leveraging nature-based mechanisms. Such naturally derived products can be food-grade and directly usable in food, nutraceutical, pharmaceutical and cosmetic products without the use of synthetic organic solvents or organic solvents derived from petroleum or petrochemical products.

Like most phenylpropanoid/benzenoid compounds, benzaldehyde biosynthesis begins with the deamination of phenylalanine to trans-cinnamic acid by the well-known enzyme phenylalanine ammonia lyase. Boatright et al. (2004), supra; and Lynch et al., Multifaceted plant responses to circumvent Phe hyperaccumulation by downregulation of flux through the shikimate pathway and by vacuolar Phe sequestration, The Plant J. 92: 939-950 (2017). The following conversion of cinnamic acid to benzaldehyde requires (i) shortening of the side chain by two carbons and (ii) introduction of an aldehyde functional group to the side chain, which can occur as an integral part of chain shortening.

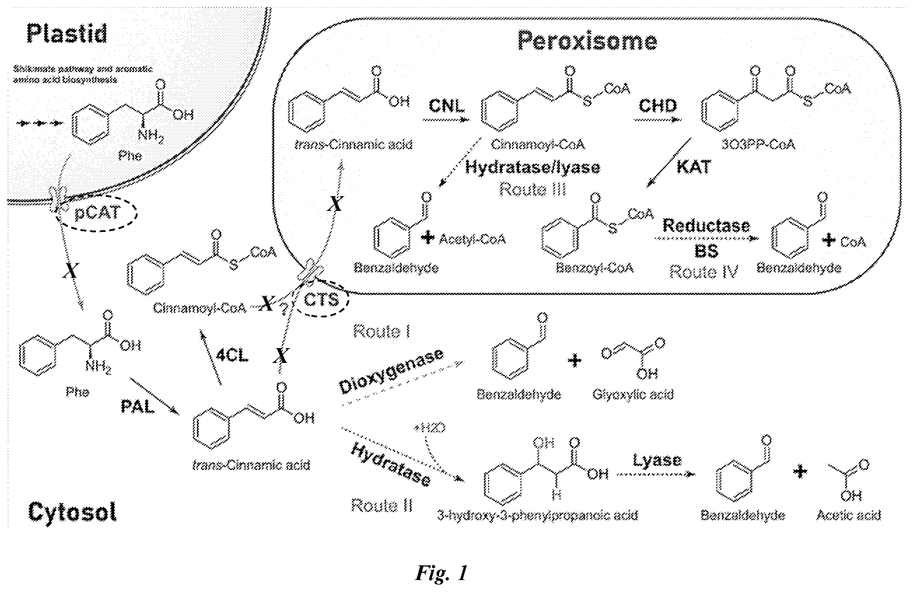

Previously, as summarized in FIG. 1, several routes have been proposed for benzaldehyde formation from cinnamic acid including both β-oxidative and non-β-oxidative pathways, and it was unknown if the non-β-oxidative pathway is CoA-dependent or CoA-independent. In the previously hypothesized CoA-independent non-β-oxidative pathway, it was proposed that benzaldehyde originates from cinnamic acid by the direct cleavage of the double bond by a putative dioxygenase—analogous to partially purified and characterized Vanilla planfolia phenylpropanoid 2,3-dioxygenase, which produces an aldehyde vanillin and glyoxylic acid from ferulic acid (see FIG. 1). Negishi and Negishi, Phenylpropanoid 2,3-dioxygenase involved in the cleavage of the ferulic acid side chain to form vanillin and glyoxylic acid in Vanilla planifolia, Bioscience Biotechnology & Biochemistry 8451: 1-9 (2017). Alternatively, a competing theory was that the double bond in the side chain of cinnamic acid underwent hydration to form 3-hydroxy-3-phenylpropanoic acid intermediate before the cleavage by a hydratase/lyase-type enzyme yielding benzaldehyde and acetate. While enzyme activity catalyzing similar non-oxidative formation of benzaldehyde from cinnamic acid was recently reported in cell culture of Asian pear Pyrus pyrifolia, heretofore a gene that encoded this enzyme had not been isolated. Saini et al., A new enzymatic activity from elicitor-treated pear cell cultures converting trans-cinnamic acid to benzaldehyde, Physiologia Plantarum 167: 64-74 (2019).

Despite the multiple potential non-β-oxidative routes for benzaldehyde biosynthesis, feeding experiments with stable isotope-labeled (2H₆, ¹⁸O)3-hydroxy-3-phenylpropanoic acid supported the existence of a β-oxidative route in cucumber (Cucumis sativus) and Nicotiana attenuata. Jarvis et al., 3-Hydroxy-3-phenylpropanoic acid is an intermediate in the biosynthesis of benzoic acid and salicylic acid but benzaldehyde is not, Planta 212: 119-126 (2000). Moreover, recent genetic studies suggested that benzaldehyde biosynthesis in plants depends on a cinnamoyl-CoA intermediate produced by peroxisomal cinnamate-CoA ligase (CNL), which catalyzes the first committed step in benzoic acid biosynthesis via the β-oxidative pathway. Sas et al. (2016), supra; Colquhoun et al., A peroxisomally localized acyl-activating enzyme is required for volatile benzenoid formation in a Petunia x hybrida cv. ‘Mitchell Diploid’ flower, J. Experimental Botany 63: 4821-4833 (2012); Amrad et al., Gain and loss of floral scent production through changes in structural genes during pollinator-mediated speciation, Current Biology 26: 3303-3312 (2016); Klempien et al., Contribution of CoA ligases to benzenoid biosynthesis in petunia flowers, The Plant Cell 24: 2015-2030 (2012); Lee et al., Benzoylation and sinapoylation of glucosinolate R-groups in Arabidopsis, The Plant J. 72: 411-422 (2012); and Widhalm and Dudareva, A familiar ring to it: Biosynthesis of plant benzoic acids, Molecular Plant 8: 83-97 (2015). Indeed, loss-of-function mutations in CNL led to the loss of benzaldehyde emission in bird-pollinated Petunia exserta and selfing Capsella rubella. Amrad et al. (2016), supra; Sas et al. (2016), supra; Jantzen et al., Retracing the molecular basis and evolutionary history of the loss of benzaldehyde emission in the genus Capsella, New Phytologist 224: 1349-1360 (2019).

It has also been conventionally contemplated that cinnamoyl-CoA could be converted directly to benzaldehyde by an enoyl-CoA hydratase/lyase (FIG. 1), thus linking the β-oxidative and non-β-oxidative pathways. While a protein fraction with an enoyl-CoA hydratase/lyase activity catalyzing the hydration and non-oxidative cleavage of cinnamoyl-CoA to benzaldehyde (probably through an enzyme-bound 3-enoyl-CoA intermediate) has been characterized from Hypericum androsaemum cell culture, the corresponding gene also remains unknown. Abd El-Mawla and Beerhues, Benzoic acid biosynthesis in cell cultures of Hypericum androsaemum, Planta 214: 727-733 (2002).

In the proposed β-oxidative route, it was proposed that cinnamoyl-CoA in peroxisomes is first converted to benzoyl-CoA by cinnamoyl-CoA hydratase/dehydrogenase (CHD) and 3-ketoacyl-CoA thiolase (KAT), followed by reduction to benzaldehyde by an enzyme similar to cinnamoyl-CoA reductase (CCR) (which catalyzes the reduction of hydroxycinnamoyl-CoA thioesters to their corresponding aldehydes in lignin biosynthesis). Widhalm and Dudareva (2015), supra; and Bonawitz and Chapple, The genetics of lignin biosynthesis: Connecting genotype to phenotype, Ann. Rev. Genetics 44: 337-363 (2010). The CCR enzymes typically exhibit broad substrate specificity and utilize p-coumaroyl-CoA, caffeoyl-CoA, feruloyl-CoA, 5-hydroxyferuloyl-CoA and sinapoyl-CoA. Pan et al., Structural studies of cinnamoyl-CoA reductase and cinnamyl-alcohol dehydrogenase, key enzymes of monolignol biosynthesis, The Plant Cell 26: 3709-3727 (2014).

Benzoyl-CoA itself is a poor substrate for most of the characterized plant CCR isoforms with exception of three out of 18 CCR family members in cucumber Cucumis sativus; however, heretofore no benzoyl-CoA specific reductase has been reported. Liu et al., Benzaldehyde synthases are encoded by cinnamoyl-CoA reductase genes in cucumber (Cucumis sativus L.), bioRxiv: doi:10.1101/2019.12.26.889071 (2019).

### Enzymes

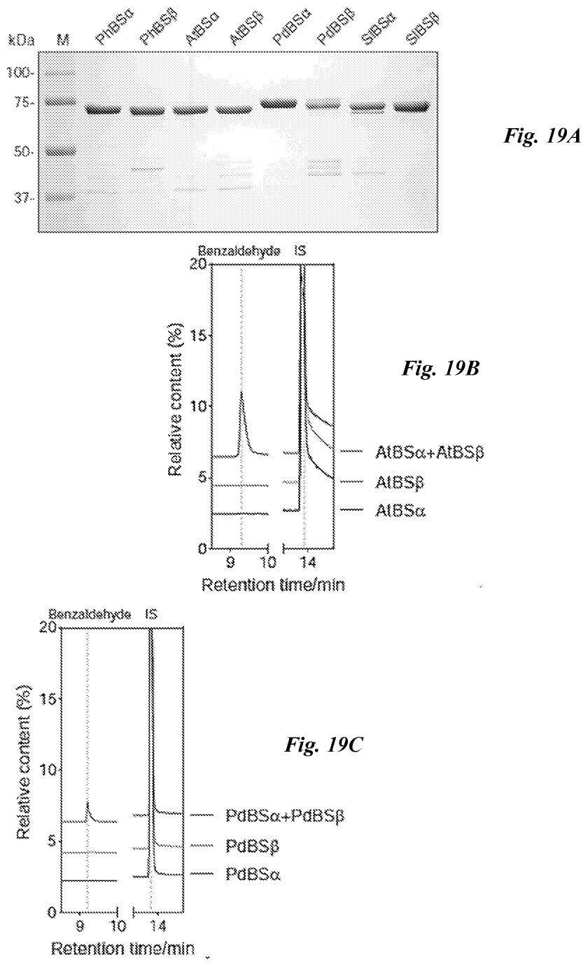

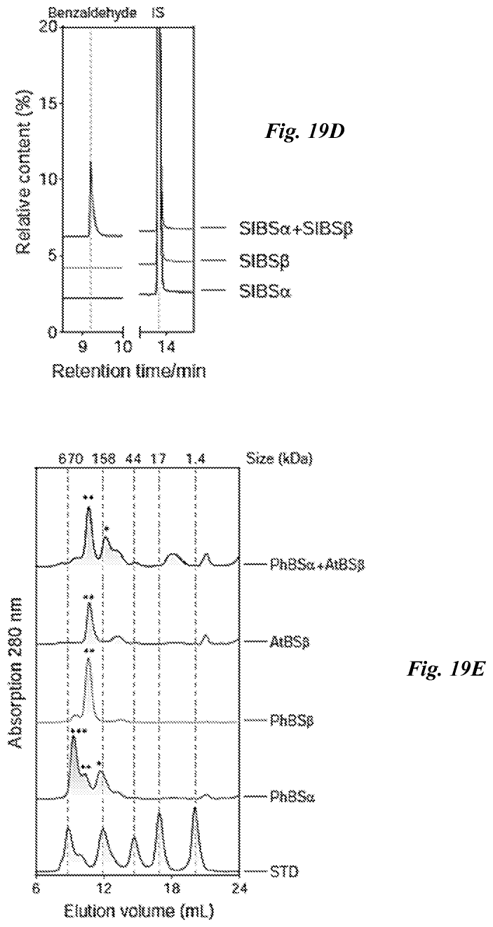

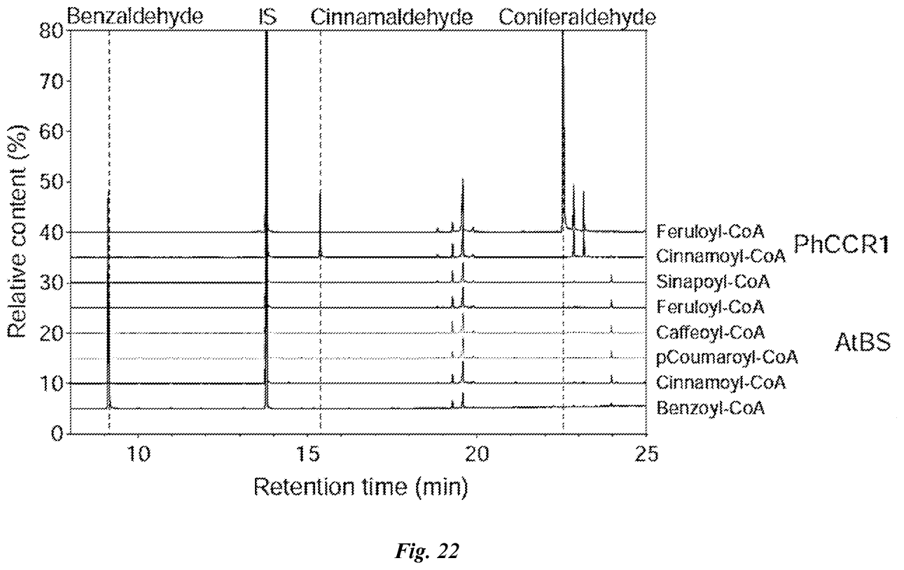

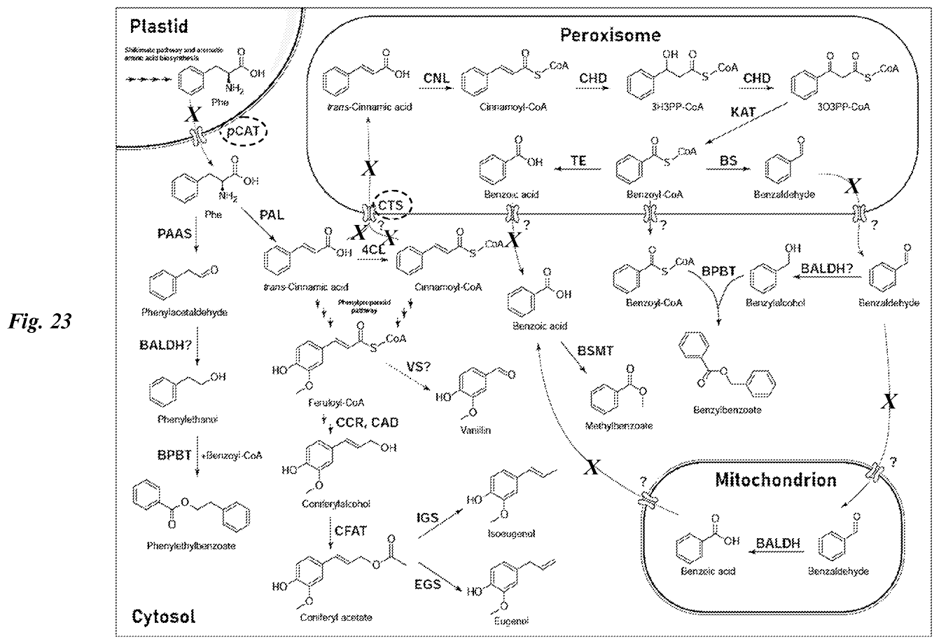

The findings presented herein provide biochemical (FIGS. 4A and 19B-19D) and genetic evidence (FIGS. 5, 14A, 14B, and 20A-20E) that a heterodimeric enzyme comprising two distinct subunits is responsible for benzaldehyde formation in plants. This enzyme can convert benzoyl-CoA to benzaldehyde using nicotinamide adenine dinucleotide phosphate (NADPH) as the reducing power and can exhibit strict substrate specificity towards benzoyl-CoA (e.g., does not accept hydroxycinnamoyl-CoA thioesters) (see FIGS. 4B and 22).

Leveraging these findings, in certain embodiments, a heterodimeric enzyme is provided (e.g., a benzaldehyde synthase (BS)) that comprises a BS alpha (BSα) subunit and a BS beta (BSβ) subunit. The BSα and BSβ subunits can be distinct from each other. The BSα subunit and the BSβ subunit can be present in a molar ratio of about 1:1. In certain embodiments, the heterodimeric enzyme is a hybrid enzyme.

The heterodimeric enzyme can exhibit substrate specificity towards benzoyl-CoA in the biosynthesis of natural or semi-natural benzaldehyde. The heterodimeric enzyme can exhibit strict substrate specificity towards benzoyl-CoA in the biosynthesis of natural or semi-natural benzaldehyde.

In certain embodiments, the BSα subunit and the BSβ subunit are both derived from the NAD(P)-binding Rossmann-fold superfamily. For example, and without limitation, the BSα and/or BSβ subunits can be from Arabidopsis, Petunia, Solanum, or Prunus. In certain embodiments, the BSα and/or BSβ subunits are from Petunia hybrida. In certain embodiments, the BSα and/or BSβ subunits are from Solanum lycopersicum. In certain embodiments, the BSα and/or BSβ beta subunits are from Prunus dulcis. In certain embodiments, the BSα and/or BSβ beta subunits are from a homolog of Petunia hybrida.

The heterodimeric enzyme can be engineered to modify expression of the BSα subunit, the BSβ subunit, or both the BSα and BSβ subunits. For example, the enzyme can be engineered to overexpress the BSβ subunit relative to the BSα subunit. In at least one embodiment, the BSα subunit is a Petunia hybrida BSα subunit and the BSβ subunit is a Petunia hybrida BSβ subunit and is upregulated to achieve a molar ratio of 1:1 between the two subunits. “Overexpression”, “upregulation”, and their variants refer to the level of expression in transgenic cells, organisms and/or a biological marker (e.g., a subunit of a protein or a biochemical product) that exceeds levels of expression in normal or untransformed (non-transgenic or wild-type) cells or organisms or in that particular marker in wild-type (i.e., an unmodified or native corresponding cell, organism, or marker). When used in the context of a gene, “overexpression” or “upregulation” can refer to an increase in the level of protein and/or mRNA product from a target gene, for example, in the range of between about 20% and about 100% as compared to wild type. In certain instances, upregulation can also result in an increase in the level of downstream metabolites of such upregulated gene or protein.

The enzyme can be peroxisomally localized similar to PhBS, or engineered to be localized in other subcellular compartments when in planta or in vivo including, without limitation, the cytosol, plastids, and/or mitochondria.

The BSα subunit can be encoded by a nucleic acid sequence of a first species, and the BSβ subunit can be encoded by a nucleic acid sequence of a second species. In certain embodiments, both the BSα and BSβ subunits of the enzyme are from a single species (i.e., the first and second species are the same). In certain embodiments, both the BSα and BSβ subunits of the enzyme can be from a single species (i.e., the first and second species are the same), but one or both of the subunits are overexpressed.

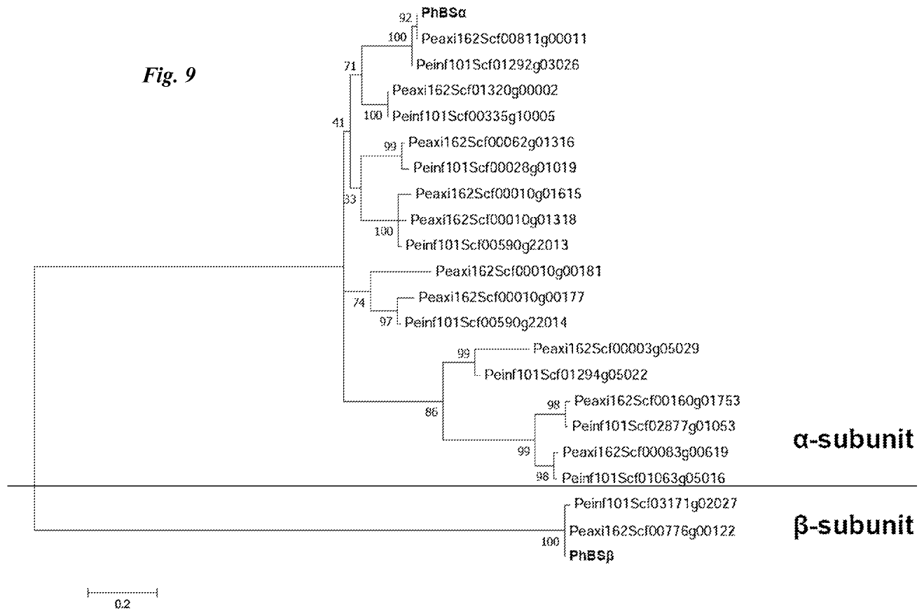

Alternatively, as noted above, the enzyme can be a hybrid enzyme. In such embodiments, the first and second nucleic acid sequences that encode the BSα and BSβ subunits, respectively, are from different species. For example, the first species and the second species can each be a species independently selected from the Arabidopsis genus, the Petunia genus, the Prunus genus, the Solanum genus, or any of the species identified in FIG. 9, 10, or 17. The first species and the second species can both be from the be independently selected from the same genus or be different species within the same genus. In certain embodiments, the first species is Petunia hybrida or Solanum lycopersicum and the second species is not within the Arabidopsis genus. Alternatively, the first species can be Arabidopsis thaliana and the second species can be Prunus dulcis, Petunia hybrida, or Solanum lycopersicum.

In certain embodiments, the first nucleic acid sequence encodes an Arabidopsis thaliana BSα, and the second nucleic acid sequence encodes a Petunia hybrida BSβ. In certain embodiments, the first nucleic acid sequence encodes a Solanum lycopersicum BSα, and the second nucleic acid sequence encodes a Solanum lycopersicum BSβ.

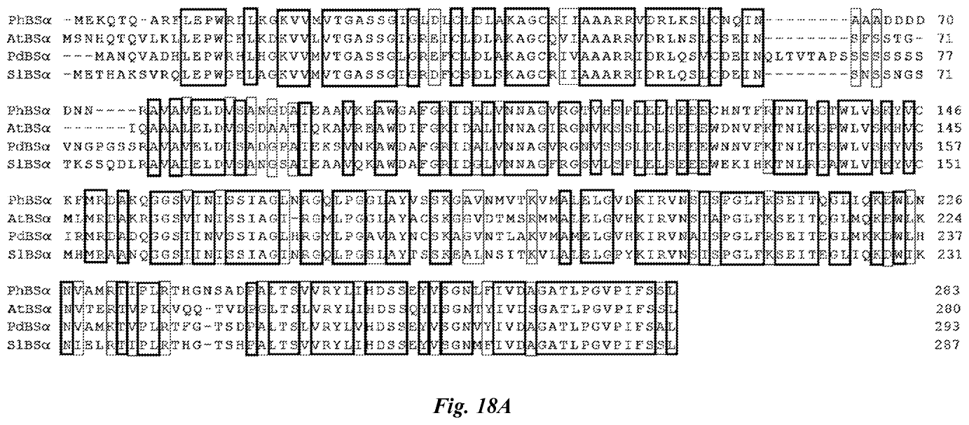

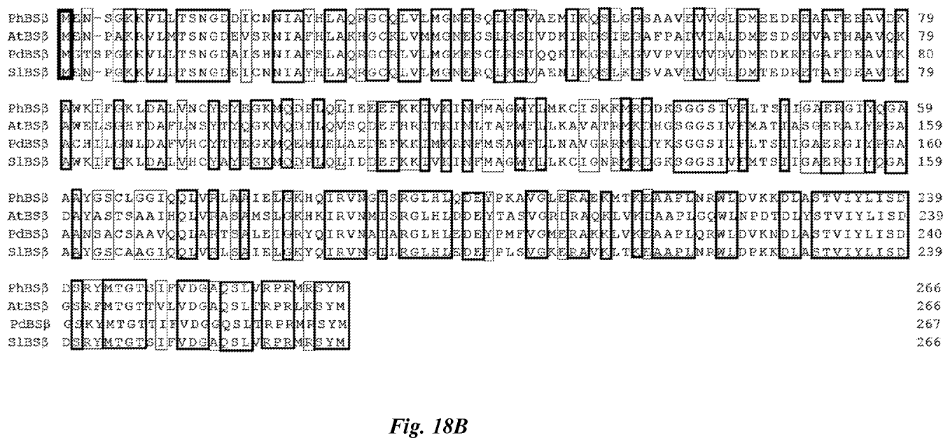

The BSα can comprise SEQ ID NO: 3 or at least 50% identity to SEQ ID NO: 3. The BSβ can comprise SEQ ID NO: 4 or at least 50% identity to SEQ ID NO: 4. “Percent identity” or “% identity” describes the extent to which polynucleotides or protein segments are invariant in an alignment of sequences, for example nucleotide sequences or amino acid sequences. As shown in FIGS. 18A and 18B, an alignment of sequences is created by manually aligning two sequences, for example, a stated sequence as a reference and another sequence, to produce the highest number of matching elements (e.g., individual nucleotides or amino acids) while allowing for the introduction of gaps into either sequence. An “identity fraction” for a sequence aligned with a reference sequence is the number of matching elements, divided by the full length of the reference sequence, not including gaps introduced by the alignment process into the reference sequence. “Percent identity” as used herein is the identity fraction times 100.

In certain embodiments, the BSα is encoded by SEQ ID NO: 101, SEQ ID NO: 103, or SEQ ID NO: 105 (or is encoded by a sequence that has at least about 50% identity therewith, at least about 60% identity therewith, or at least about or greater than 70% identity therewith). In certain embodiments, the BSβ is encoded by SEQ ID NO: 102, SEQ ID NO: 104, or SEQ ID NO: 106 (or is encoded by a sequence that has at least about 50% identity therewith, at least about 60% identity therewith, or at least about or greater than 70% identity therewith).

A “homolog” or “homologs” means a protein in a group of proteins that perform the same biological function, for example, proteins that belong to the same protein family and that provide a common enhanced trait (e.g., in the transgenic plants provided herein). Homologs can be expressed by homologous genes. With reference to homologous genes, homologs include orthologs (i.e., genes expressed in different species that evolved from a common ancestral gene by speciation and encode proteins that retain the same function), but do not include paralogs (i.e., genes that are related by duplication but have evolved to encode proteins with different functions). Homologous genes include naturally occurring alleles and artificially created variants. Degeneracy of the genetic code provides the possibility to substitute at least one base of the protein encoding sequence of a gene with a different base without causing the amino acid sequence of the polypeptide produced from the gene to be changed. When optimally aligned, homolog proteins, or their corresponding nucleotide sequences, have typically at least about 50% identity, at least about 60% identity, in some instances at least about 70%, at least about 75%, at least about 80%, at least about 85%, at least about 90%, at least about 92%, at least about 94%, at least about 95%, at least about 96%, at least about 97%, at least about 98%, at least about 99%, and even at least about 99.5% identity over the full length of a protein or its corresponding nucleotide sequence identified as being associated with imparting an enhanced trait when expressed in plant cells or another organism. In one aspect of the disclosure, homolog proteins have at least about 50%, least about 55%, at least about 60%, at least about 65%, at least about 70%, at least about 75%, at least about 80%, at least about 85%, at least about 90%, at least about 92%, at least about 94%, at least about 95%, at least about 96%, at least about 97%, at least about 98%, at least about 99%, and at least about 99.5% identity to a consensus amino acid sequence of proteins and homologs that can be built from sequences disclosed herein.

Homologs are inferred from sequence similarity, by comparison of protein sequences, for example, manually or by use of a computer-based tool using known sequence comparison algorithms such as BLAST and FASTA. A sequence search and local alignment program (e.g., BLAST) can be used to search query protein sequences of a base organism against a database of protein sequences of various organisms, to find similar sequences, and the summary Expectation value (E-value) can be used to measure the level of sequence similarity. A further aspect of the homologs encoded by DNA useful in the transgenic plants hereof are those proteins that differ from a disclosed protein as the result of deletion or insertion of one or more amino acids in a native sequence.

Other functional homolog proteins differ in one or more amino acids from those of a trait-improving protein disclosed herein as the result of one or more of known conservative amino acid substitutions; for example, valine is a conservative substitute for alanine and threonine is a conservative substitute for serine. Conservative substitutions for an amino acid within the native sequence can be selected from other members of a class to which the naturally occurring amino acid belongs. Representative amino acids within these various classes include, but are not limited to: (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, tyrosine, asparagine, and glutamine; and (4) neutral nonpolar (hydrophobic) amino acids such as alanine, leucine, isoleucine, valine, proline, phenylalanine, tryptophan, and methionine. Conserved substitutes for an amino acid within a native protein or polypeptide can be selected from other members of the group to which the naturally occurring amino acid belongs. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side 30 chains is cysteine and methionine. Naturally conservative amino acids substitution groups are valine-leucine, valine-isoleucine, phenylalanine-tyrosine, lysine-arginine, alanine-valine, aspartic acid-glutamic acid, and asparagine-glutamine. A further aspect of the disclosure includes proteins that differ in one or more amino acids from those of a described protein sequence as the result of deletion or insertion of one or more amino acids in a native sequence.

Benzaldehyde synthases are widespread in the plant kingdom. While plant genomes contain multiple copies of genes encoding a subunit homologs, most species have only a single copy of a β subunit gene (FIGS. 9, 10, and 17). Nevertheless, the findings hereof support that a homolog of, for example, Petunia hybrida BS (e.g., the homologs listed in FIGS. 9, 10, and 17) and the subunits thereof can be, where desired, used interchangeably with Petunia hybrida BS.

### Transgenic Plants

Novel transgenic plants are also provided that produce the enzymes hereof The transgenic plants can leverage the novel findings described herein to produce active and natural or semi-natural benzaldehydes (e.g., the enzymes hereof) and other downstream products in amounts significantly greater than those produced by a corresponding wild-type plant and/or as heretofore been possible using conventional methodologies. The biological mechanisms in planta can be directed to provide efficient benzaldehyde production. The existence of efficient benzaldehyde reducing capacities in planta can further be used to produce downstream benzaldehyde derivatives (e.g., benzylalcohol) as desired.

In at least one embodiment, the transgenic plants, platforms, and inventive methods provide for at least a 6-fold increase in benzaldehyde production as compared to a corresponding wild-type plant. In certain embodiments, the benzaldehyde production of the transgenic plant is at least 4-fold greater than benzaldehyde production in a corresponding wild-type plant. In certain embodiments, the benzaldehyde production of the transgenic plant is at or near 7.8-fold greater than benzaldehyde production in a corresponding wild-type plant.

The term “transgenic plant” refers herein to a plant, plant tissue, or plant cell whose genome has been altered by the stable integration of recombinant DNA. For example, and without limitation, a transgenic plant can have incorporated DNA sequences including, but not limited to, one or more genes that are perhaps not normally present, one or more genes or DNA sequences that are upregulated or downregulated as compared to wild-type expression in a like native plant, DNA sequences not normally transcribed into RNA or translated into a protein (“expressed”), or any other genes or DNA sequences that one desires to introduce into the non-transformed plant, but which one desires to either genetically engineer or to have altered expression. “Transformation” refers to a process of introducing an exogenous nucleic acid sequence (vector or construct, for example) into a cell or protoplast in which that exogenous nucleic acid is incorporated into the nuclear DNA, plastid DNA, or is otherwise capable of autonomous replication. Notably, as used herein, the term “plant” means and includes an entire plant, a portion of a plant (e.g., roots and leaves, etc.), a plant tissue or a portion thereof, or one or more plant cells, unless otherwise expressly specified.

It is contemplated that in some instances the genome of the transgenic plants hereof will have been augmented through the stable introduction of the transgene; however, in other instances, an introduced gene will replace an endogenous sequence. A transgenic plant includes a plant regenerated from an originally transformed plant or plant cell and progeny transgenic plants from later generations or crosses of a transgenic plant. “Regeneration” refers to the process of growing a plant from a plant cell, and “regeneration medium” means a plant tissue culture medium required for containing a selection agent.

The transgenic plant can be any species of plant capable of expressing the nucleic acid sequences hereof. For example, the transgenic plant can be Petunia hybrida, Nicotiana benthamiana, or Prunus dulcis. While certain examples and embodiments describe a transgenic plant to be of a particular species, it will be appreciated that this is not limiting, but rather one of skill in the art will appreciate the concepts hereof can be applied to numerous plant species.

The transgenic plant can comprise a first nucleic acid sequence encoding a BSα subunit and a second nucleic acid sequence encoding a BSβ subunit (e.g., of the heterodimeric enzyme described above).

One or both of the first and second nucleic acid sequences can be operably linked to a regulatory element (e.g., a promoter) for directing expression of the first and/or second nucleic acid sequences. The term “operably linked” means a first polynucleotide molecule, such as a promoter, connected with a second transcribable polynucleotide molecule, such as a gene of interest, where the polynucleotide molecules are so arranged that the first polynucleotide molecule affects the function of the second polynucleotide molecule. The two polynucleotide molecules can or need not be part of a single contiguous polynucleotide molecule and can (but need not) be adjacent. For example, a promoter is operably linked to a gene of interest if the promoter modulates transcription of the gene of interest in a cell.

Regulatory sequences are common to the person of the skill in the art and can include, for example, an origin of replication, a promoter sequence, and/or an enhancer sequence. The polynucleotide encoding the desired protein can exist extrachromosomally or can be integrated into the host cell chromosomal DNA. Promoters, leaders, enhancers, introns, transit, or targeting or signal peptides, and 3′ transcriptional termination regions are genetic elements that can be operably linked in an expression construct.

In certain embodiments, the transgenic plant is engineered to modify expression of the BSα subunit, the BSβ subunit, or both the BSα and BSβ subunits. Expression of both the first and second nucleic acid sequences can be upregulated such that the transgenic plant overexpresses both the BSα and BSβ subunits as compared to a corresponding wild-type plant. For example, expression of the first and/or second nucleic acid sequences can be upregulated (e.g., via incorporation of a regulatory element such as a promoter or the like) such that the transgenic plant overexpresses at least the BSβ subunit and/or both the BSα and BSβ subunits.

In certain embodiments, both the first and second nucleic acid sequences are operably linked to one or more regulatory elements that upregulate production of the BSα and BSβ subunits such that both BSα and BSβ subunits are overexpressed in the transgenic plant. In certain embodiments, only the second nucleic acid sequence is operably linked to one or more regulatory elements that upregulate production of the BSβ subunit such that the BSβ subunit is overexpressed in the transgenic plant. In at least one embodiment, expression of the BSα subunit and/or the BSβ subunit are modified (e.g., upregulated) to achieve a molar ratio of 1:1 between the two subunits.

The regulatory element can, in certain instances, comprise a tissue-specific promoter for directing expression of the first and/or second nucleic acid sequence in the plant cells of a leaf, root, flower, developing ovule or seed of the transgenic plant.

Now referring to the nucleic acid sequences that encode the BSα and BSβ subunits, such nucleic acid sequences can be heterologous to the transgenic plant. In certain embodiments, the BSα subunit and the BSβ subunit are both from the NAD(P)-binding Rossmann-fold superfamily. For example, and without limitation, the BSα and/or BSβ subunits can be from Arabidopsis, Petunia, Solanum, or Prunus.

In certain embodiments, the BSα and/or BSβ subunits are from (or obtained from) Petunia hybrida. In certain embodiments, the BSα and/or BSβ subunits are from Solanum lycopersicum. In certain embodiments, the BSα and/or BSβ beta subunits are from Prunus dulcis. In certain embodiments, the BSα and/or BSβ beta subunits are from a homolog of Petunia hybrida.

The first nucleic acid sequence can be from a first species, and the second nucleic acid sequence can be from a second species. In certain embodiments, both the BSα and BSβ subunits are from a single species (i.e., the first and second species are the same). Both BSα and BSβ subunits can be from a single species (i.e., the first and second species are the same), but one or both of the subunits are overexpressed.

In certain embodiments, the first and second nucleic acid sequences that encode the BSα and BSβ subunits, respectively, are from different species. For example, the first species and the second species can each be a species independently selected from the Arabidopsis genus, the Petunia genus, the Prunus genus, the Solanum genus, or any of the species identified in FIG. 9, 10, or 17. The first species and the second species can both be independently selected from the same genus or be different species within the same genus. In certain embodiments, the first species is Petunia hybrida or Solanum lycopersicum, and the second species is not within the Arabidopsis genus. Alternatively, the first species can be Arabidopsis thaliana, and the second species can be Prunus dulcis, Petunia hybrida, or Solanum lycopersicum.

In certain embodiments, the first nucleic acid sequence encodes an Arabidopsis thaliana BSα, and the second nucleic acid sequence encodes a Petunia hybrida BSβ subunit. In certain embodiments, the first nucleic acid sequence encodes a Solanum lycopersicum BSα subunit, and the second nucleic acid sequence encodes a Solanum lycopersicum BSβ subunit.

The first nucleic acid sequence can be or comprise a nucleotide sequence of SEQ ID NO: 3 or a nucleotide sequence that has at least 50% identity to SEQ ID NO: 3 and encodes a BSα subunit. The second nucleic acid sequence can be or comprise a nucleotide sequence of SEQ ID NO: 4 or a nucleotide sequence that has at least 50% identity to SEQ ID NO: 4 and encodes a BSβ subunit. In certain embodiments, the first nucleic acid sequence is or comprises a nucleotide sequence of SEQ ID NO: 101, SEQ ID NO: 103, or SEQ ID NO: 105 (or a nucleotide sequence that has at least about 50% identity therewith, at least about 60% identity therewith, or at least about or greater than 70% identity therewith) and encodes a BSα subunit. In certain embodiments, the second nucleic acid sequence is or comprises a nucleotide sequence of SEQ ID NO: 102, SEQ ID NO: 104, or SEQ ID NO: 106 (or a nucleotide sequence that has at least about 50% identity therewith, at least about 60% identity therewith, or at least about or greater than 70% identity therewith) and encodes a BSβ subunit.

The first nucleic acid sequence can encode SEQ ID NO: 1, SEQ ID NO: 5, SEQ ID NO: 6, SEQ ID NO: 7 or a functional fragment or homolog of SEQ ID NO: 1, SEQ ID NO: 5, SEQ ID NO: 6, or SEQ ID NO: 7. The second nucleic acid sequence can encode SEQ ID NO: 2, SEQ ID NO: 8, SEQ ID NO: 9, SEQ ID NO: 10, or a functional fragment or homolog of SEQ ID NO: 2, SEQ ID NO: 8, SEQ ID NO: 9, or SEQ ID NO: 10.

In certain embodiments, the first nucleic acid sequence encodes SEQ ID NO: 1, SEQ ID NO: 5, SEQ ID NO: 6, SEQ ID NO: 7 or a functional fragment or homolog of SEQ ID NO: 1, SEQ ID NO: 5, SEQ ID NO: 6, or SEQ ID NO: 7; and the second nucleic acid sequence encodes SEQ ID NO: 2, SEQ ID NO: 8, SEQ ID NO: 9, SEQ ID NO: 10, or a functional fragment or homolog of SEQ ID NO: 2, SEQ ID NO: 8, SEQ ID NO: 9, or SEQ ID NO: 10.

### Methods for Producing Benzaldehyde

Methods are also provided for producing benzaldehyde (e.g., natural or semi-natural benzaldehyde). Such methods can utilize the novel enzymes, nucleic acid sequences and combinations thereof, and/or transgenic plants hereof.

In certain embodiments, a method for producing benzaldehyde comprises providing a biosynthesis platform is provided. The biosynthesis platform can comprise a first nucleic acid sequence encoding a BSα subunit and a second nucleic acid sequence encoding a BSβ subunit. In certain embodiments, the first and/or second nucleic acid sequences are overexpressed in the biosynthesis platform. The method further comprises subjecting the biosynthesis platform to conditions such that benzaldehyde is produced. Benzaldehyde can thereafter be isolated from the biosynthesis platform. Such methods can further comprise purification steps as desired.

The first and second nucleic acid sequences can be any of those (or combinations of those) described herein. Expression of these nucleic acid sequences and the subsequent combination of the subunits results in the heterodimeric enzyme described herein that is active. In certain embodiments, the BSα and BSβ subunits are produced independently of each other, and the method further comprises combining the BSα and BSβ subunits. Alternatively, the BSα and BSβ subunits can be produced in the same population of microbes or transgenic plant as is described in additional detail below. In certain embodiments, the BSα subunit and BSβ subunit are present in a molar ratio of about 1:1 when combined (whether such combination is an active combination step or simply by virtue of the subunits both being produced in the same population or cells).

The method can further comprise isolating the benzaldehyde or other downstream products from the biosynthesis platform (e.g., benzylalcohol).

Benzoyl-CoA, NADPH, or both benzoyl-CoA and NADPH can be supplied to the biosynthesis platform. In certain embodiments, such addition(s) can facilitate (i.e., increase) production of benzaldehyde.

The biosynthesis platform can be a genetically engineered plant (e.g., a transgenic plant, a transgenic plant cell, and/or a transgenic plant part (e.g., an organ) or tissue), one or more genetically engineered eukaryotic cells, tissues, organs or organisms (e.g., algae, insect, or animal cells), or one or more microorganisms or microbes. In certain embodiments, where a biosynthesis platform comprises a transgenic plant, the transgenic plant can be genetically engineered to upregulate production of benzoyl-CoA and/or NADPH.

In certain embodiments, the biosynthesis platform is transformed with a vector carrying the nucleic acid sequences hereof under conditions that allow for the overexpression of the BSα and/or BSβ subunit(s) (e.g., by the transgenic plant). In certain embodiments, the biosynthesis platform is transformed with a first vector carrying the nucleic acid sequences hereof under conditions that allow for the overexpression of the BSα and/or BSβ subunit(s), and a second vector carrying a regulatory element and/or other nucleic acid sequence that upregulates production of benzoyl-CoA and/or NADPH in the plant.

The nucleic acid sequences can be transformed into the biosynthesis platform pursuant to methods well-known in the art. In certain embodiments, the genetic components (e.g., the first and second nucleic acid sequences) are incorporated into a DNA composition such as a vector. The vector can be any molecule that can be used as a vehicle to transfer genetic material into a cell. Examples of vectors include plasmids (e.g., double-stranded plasmids), viral vectors, autonomously replicating sequences, recombinants, phages, cosmids, artificial chromosomes, and any linear or circular single- or double-stranded DNA or RNA nucleotide segment derived from any source, capable of genomic integration or autonomous replication, comprising a nucleic acid molecule in which one or more nucleic acid sequences can be linked in a functionally operative manner.

Examples of molecular biology techniques used to transfer nucleotide sequences into a microorganism or other cell include, without limitation, transfection, electroporation, transduction, and transformation. Insertion of a vector into a target cell is usually called transformation for bacterial cells and transfection for eukaryotic cells, however insertion of a viral vector is often called transduction. The terms transformation, transfection, and transduction are used interchangeably herein. These methods are well-known in the art.

The (poly)nucleotide encoding the desired enzyme can be endogenous or heterologous to the host cell. In certain embodiments, the polynucleotide is introduced into the cell using a vector; however, naked DNA can also be used. The polynucleotide can be circular or linear, single-stranded or double-stranded, and can be DNA, RNA, or any modification or combination thereof.

In certain embodiments, the vector is an expression vector. An “expression vector” or “expression construct” is any vector or cassette that can be used to introduce a specific (poly)nucleotide into a target cell such that once the expression vector is inside the cell, the protein encoded by the polynucleotide is produced by the cellular transcription and translation machinery. Typically, an expression vector includes regulatory sequences operably linked to the polynucleotide encoding the desired protein.

As will be appreciated by a person of skill in the art, overexpression of an enzyme can be achieved through a number of molecular biology techniques. For example, overexpression can be achieved by introducing into the host cell one or more copies of a polynucleotide encoding the desired enzyme. Transcription of DNA into mRNA is regulated by a region of DNA usually referred to as the “promoter.” The promoter region contains a sequence of bases that signals RNA polymerase to associate with the DNA and to initiate the transcription into mRNA using one of the DNA strands as a template to make a corresponding complementary strand of RNA.

Promoters are generally known it the art. A number of promoters that are active in plant cells include, without limitation, nopaline synthase (NOS) and octopine synthase (OCS) promoters that are carried on tumor-inducing plasmids of Agrobacterium tumefaciens, the caulimovirus promoters such as the cauliflower mosaic virus (CaMV) 19S and 35S promoters, and the figwort mosaic virus (FMV) 35S promoter, the enhanced CaMV35S promoter (e35S), and the light-inducible promoter from the small subunit of ribulose bisphosphate carboxylase (ssRUBISCO). Promoter hybrids can also be constructed to enhance transcriptional activity or to combine desired transcriptional activity, inducibility and tissue specificity or developmental specificity. Promoters that function in plants include, without limitation, promoters that are inducible, viral, synthetic, and temporally regulated, spatially regulated, and spatio-temporally regulated. Other promoters that are tissue-enhanced, tissue-specific or developmentally regulated are also known in the art.

The promoters used in the methods hereof can be modified, if desired, to affect their control characteristics. Promoters can be derived by means of ligation with operator regions, random or controlled mutagenesis, etc. Further, the promoters can be altered to contain multiple “enhancer sequences” to facilitate elevating gene expression.

The nucleic acids that can be introduced by the methods hereof can include, for example, DNA sequences or genes from another species (i.e., as compared to the biosynthesis platform), or genes or sequences that originate with or are present in the same species (i.e., as compared to the biosynthesis platform), but are incorporated into recipient cells by genetic engineering methods rather than classical reproduction or breeding techniques. “Heterologous” refer to genes or DNA that are not necessarily present in the cell being transformed, or simply not present in the form, structure, etc. as found in the transforming DNA segment or gene, or genes that are normally present yet that one desires, for example, to have overexpressed. Thus, term “heterologous” gene or sequence refers to any gene or DNA segment that is introduced into a recipient cell, regardless of whether a similar gene may already be present in such a cell. The type of DNA included in the heterologous DNA can include DNA that is already present in the host cell, DNA from another species (e.g., another plant where the biosynthesis platform comprises a transgenic plant), DNA from a different organism, or a DNA generated externally such as a DNA sequence containing an antisense message of a gene, or a DNA sequence encoding a synthetic or modified version of a gene.

The technologies for the introduction of DNA into cells are well-known to those of skill in the art and include, without limitation: (1) chemical methods; (2) physical methods such as microinjection, electroporation, and micro-projectible bombardment; (3) vectors; (4) receptor-mediated mechanisms; and (5) bacterial (e.g., Agrobacterium)-mediated plant transformation methods. For Agrobacterium-mediated transformation, for example, after the construction of the plant transformation vector or construct, the nucleic acid molecule, prepared as a DNA composition in vitro, can be introduced into a suitable host such as E. coli and mated into another suitable hosted such as Agrobacterium, or directly transformed into component Agrobacterium. The use of various bacterial strains to introduce one or more genetic components into plants can be used.

In certain embodiments, the method comprises: transforming eukaryotic cells or microbes with a vector carrying the first nucleic acid sequence under conditions that allow for the overexpression of the BSα; transforming eukaryotic cells or microbes with a vector carrying the second nucleic acid sequence under conditions that allow for the overexpression of the BSβ; selecting transformants that overexpress both BSα and BSβ; and growing the transformants to facilitate de novo production of benzaldehyde in the biosynthesis platform.

The biosynthesis platform can comprise microbes or bacteria. In certain embodiments, the biosynthesis platform comprises genetically engineered microbes of an Escherichia coli strain, a Saccharomyces cerevisiae strain, or a Pichia pastoris strain in a fermentation medium. Isolating the produced benzaldehyde can comprise recovering the benzaldehyde from the fermentation medium after fermentation.

Where the biosynthesis platform comprises microbes, both the first and second nucleic acid sequences can be transformed into the same population of microbial cells or multiple cohorts can be employed. For example, and without limitation, a first population of microbes can be transformed with a vector carrying the first nucleic acid sequence (e.g., that encodes a BSα subunit) and a second population of microbes can be transformed with a vector carrying the second nucleic acid sequence (e.g., that encodes a BSβ subunit). In such embodiments, transformants that overexpress BSα can be selected from the first population of microbes, transformants that overexpress BSβ can be selected from the second population of microbes, and the BSα from the first population of microbes can be mixed with the second population of microbes (or vice versa) to produce benzaldehyde.

Alternatively, a single population (e.g., of microbes) can be transformed to express both the first and second nucleic acid sequences such that the single population produces both BSα and/or BSβ subunits. There, a mixing step may not be required.

Benzoyl-CoA, NADPH, or both benzoyl-CoA and NADPH can be added to the mixture of the BSα and BSβ subunits (whether such mixture is within the same population or after combination of distinctly produced BSα and BSβ subunits.

The biosynthesis platform can alternatively comprise a transgenic plant, a transgenic plant cell, and/or a transgenic plant tissue or part. In such embodiments, isolating the benzaldehyde from the biosynthesis platform can comprise isolating the benzaldehyde from the transgenic plant, a transgenic plant cell, and/or a transgenic plant tissue or part after growth.

Derivatives and downstream products of benzaldehyde can also be isolated from the biosynthesis platforms hereof In addition, the biochemical products (such as benzaldehyde, benzylalcohol, etc.) produced by the methods hereof can be purified using methods generally known in the art.

Various techniques and mechanisms of the present disclosure will sometimes describe a connection or link between two components. Words such as attached, linked, coupled, connected, and similar terms with their inflectional morphemes are used interchangeably, unless the difference is noted or made otherwise clear from the context. These words and expressions do not necessarily signify direct connections but include connections through mediate components and devices. It should be noted that a connection between two components does not necessarily mean a direct, unimpeded connection, as a variety of other components may reside between the two components of note. Consequently, a connection does not necessarily mean a direct, unimpeded connection unless otherwise noted.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference. In the event of inconsistent usages of terms between this document and those documents so incorporated by reference, the usage in the incorporated reference should be considered supplementary to that of this document; for irreconcilable inconsistencies, the usage in this document controls.

Further, any use of section headings is intended to aid reading of the document and is not to be interpreted as limiting. Further, information that is relevant to a section heading may occur within or outside of that particular section.

### Certain Definitions

As used herein, the following terms and phrases shall have the meanings set forth below. Unless defined otherwise, all technical and scientific terms used herein have the same meaning as commonly understood to one of ordinary skill in the art. In addition, it is to be understood that the phraseology or terminology employed herein, and not otherwise defined, is for the purpose of description only and not of limitation.

The term “about” can allow for a degree of variability in a value or range, for example, within 10%, within 5%, or within 1% of a stated value or of a stated limit of a range. In the present disclosure the term “substantially” can allow for a degree of variability in a value or range, for example, within 90%, within 95%, 99%, 99.5%, 99.9%, 99.99%, or at least about 99.999% or more of a stated value or of a stated limit of a range.

The terms “a,” “an,” or “the” are used herein to include one or more than one unless the context clearly dictates otherwise. The term “or” is used to refer to a nonexclusive “or” unless otherwise indicated. Thus, for example, reference to “a tRNA” includes a combination of two or more tRNAs; reference to “bacteria” includes mixtures of bacteria, and the like.

As used herein, the terms “gene overexpression” and “overexpression” (when used in connection with a gene) have the meaning ascribed thereto by one of ordinary skill in the relevant arts, which includes (without limitation) the overexpression or misexpression of a wild-type gene product that may cause mutant phenotypes and/or lead to abundant target protein expression.

“Nucleic acid” refers to deoxyribonucleotides or ribonucleotides and polymers thereof in either single- or double-stranded form and complements thereof. The term encompasses nucleic acids containing known nucleotide analogs or modified backbone residues or linkages, that are synthetic, naturally occurring, and non-naturally occurring, have similar binding properties as the reference nucleic acid, and metabolized in a manner similar to the reference nucleotides. Examples of such analogs include, without limitation, phosphorothioates, phosphoramidates, methyl phosphonates, chiral-methyl phosphonates, 2-O-methyl ribonucleotides, and peptide-nucleic acids.

The terms “polypeptide,” “peptide,” and “protein” are used interchangeably herein (unless otherwise indicated) to refer to a molecule composed of one or more chains of amino acid residues, a polypeptide, or a fragment of a polypeptide, peptide, or fusion polypeptide. The terms apply to amino acid polymers in which one or more amino acid residue is an artificial chemical mimetic of a corresponding naturally occurring amino acid, as well as to naturally occurring proteins.

The term “amino acid” refers to naturally occurring and synthetic amino acids, as well as amino acid analogs and amino acid mimetics that function in a manner similar to the corresponding naturally occurring amino acids. Naturally occurring amino acids are those encoded by the genetic code, as well as those amino acids that are later modified, e.g., hydroxyproline, γ-carboxyglutamate, and O-phosphoserine. Amino acid analog refers to a compound that has the same basic chemical structure as a naturally occurring amino acid, i.e., a carbon that is bound to a hydrogen, a carboxyl group, an amino group, and an R group (e.g., homoserine, norleucine, methionine sulfoxide, methionine methyl sulfonium). Such analogs can have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

The term “regulatory element” means and includes, in its broadest context, a polynucleotide molecule having gene regulatory activity, for example, one that has the ability to affect the transcription or translation of an operably linked transcribable polynucleotide molecule. Regulatory elements can comprise a series of nucleotides that determines if, when, and at what level a particular gene is expressed. Regulatory elements such as promoters, leaders, introns and transcription termination regions are polynucleotide molecules having gene regulatory activity that can play an integral part in the overall expression of genes in living cells. Promoters can be derived from a classical eukaryotic genomic gene, including (without limitation) the TATA box often used to achieve accurate transcription initiation, with or without a CCAAT box sequence and additional regulatory or control elements (i.e., upstream activating sequences, enhancers, and silencers) or can be the transcriptional regulatory sequences of a classical prokaryotic gene.

The terms “promote,” “promoter”, and “promoter region” refers to a synthetic or fusion molecule, or derivative thereof, that controls (e.g., confers, activates, or enhances) expression of a nucleic acid molecule in a cell, tissue, or organ. Promoters are typically found 5′ to a coding sequence and can contain additional copies of one or more specific regulatory elements to further enhance expression and/or to alter the spatial expression and/or temporal expression of a nucleic acid molecule, or to confer expression of a nucleic acid molecule to specific cells or tissues such as meristems, callus, cotyledons, leaves, roots, embryos, flowers, seeds or fruits (i.e., a tissue-specific promoter). In certain embodiments, a promoter is a plant-expressible promoter sequence, meaning that the promoter sequence (including any additional regulatory elements added thereto or contained therein) is at least capable of inducing, conferring, activating, or enhancing expression in a plant cell, tissue or organ. Promoters that also function or solely function in non-plant cells such as bacteria, yeast cells, insect cells, and animal cells, however, are not excluded from the invention hereof.

“Coding sequence,” “coding region,” or “open reading frame” refers to a region of continuous sequential nucleic acid triplets encoding a protein, polypeptide, or peptide sequence.

A cell that has been genetically engineered to express one or more metabolic enzyme(s) and/or to disrupt expression of one or more metabolically active genes as described herein is referred to as a “host” cell, a “recombinant” cell, a “genetically engineered” cell, or an “engineered” cell. For example, a genetically engineered cell can contain one or more artificial and/or heterologous sequences of nucleotides that have been created through standard molecular cloning techniques to bring together genetic material that is not natively found together. DNA sequences used in the construction of recombinant DNA molecules can originate from any species. For example, plant DNA can be joined to bacterial DNA, or human DNA can be joined with fungal DNA. Alternatively, DNA sequences that do not occur anywhere in nature can be created by the chemical synthesis of DNA and incorporated into recombinant molecules. Proteins that result from the expression of recombinant DNA are often termed recombinant proteins. Examples of recombination are commonly known in the relevant arts and can include, for example, inserting foreign polynucleotides (e.g., obtained from another species of cell) into a cell, inserting synthetic polynucleotides into a cell, or relocating or rearranging polynucleotides within a cell. Any form of recombination can be considered to be genetic engineering and, therefore, any recombinant cell can also be considered to be a genetically engineered cell. Additionally or alternatively, a genetically engineered cell can contain one or more genetic mutations that alter (e.g., disrupt or enhance) at least one normal cellular activity. For example, a microbe that contains a gene knockout is a genetically engineered organism even if it does not contain any artificial nucleotide sequences. In certain embodiments, a genetically engineered cell can be engineered to modify or alter one or more particular metabolic pathways so to cause a change in metabolism. The goal of metabolic engineering can be to improve the rate and conversion of a substrate into a desired product. General laboratory methods for introducing and expressing or overexpressing native and non-native proteins such as enzymes in many different cell types (including bacteria and plants) are routine and well-known in the art. Metabolic pathway modifications can take any number of different forms including, without limitation, modifications that reduce, attenuate, disrupt, lessen, downregulate or eliminate the expression of a metabolic enzyme, or upregulate the expression of endogenous (i.e., native to the wild-type cell) or exogenous (not native to the wild-type cell) enzymes, or that introduce new (non-native) enzymes, including non-native biosynthetic pathways for metabolic precursors or intermediates, into the cell.

“Downregulation” refers to a level of expression in transgenic cells or organisms that is lower than the levels of expression in normal or untransformed (non-transgenic or wild-type) cells or organisms. In particular, “downregulation” refers to a decrease in the level of protein and/or mRNA product from a target gene, for example, in the range of between about 20% and about 100% as compared to wild-type.

A “functional fragment” refers to a portion of a polypeptide that retains full or partial molecular, physiological, or biochemical function of the full-length polypeptide. A functional fragment often contains the domain(s) identified in the polypeptide provided in the sequence listing.

It is intended that that the scope of the present methods and compositions be defined by the following claims. However, it must be understood that this disclosure can be practiced otherwise than is specifically explained and illustrated without departing from its spirit or scope. It should be understood by those skilled in the art that various alternatives to the embodiments described herein can be employed in practicing the claims without departing from the spirit and scope as defined in the following claims.

### EXAMPLES

The following examples illustrate certain specific embodiments of the invention and are not meant to limit the scope of the invention in any way.

### Materials and Methods

### Plant Materials

Petunia hybrida cv. Mitchell diploid (W115, Ball Seed Co., Oxford, Canada) plants were grown under standard greenhouse condition with a light period from 6:00 to 21:00 hour. To link the simultaneous downregulation of PhBSα and PhBSβ6 genes via virus induced gene silencing (VIGS) to the silencing of a marker gene, a previously generated pTRV2-PDS construct containing a 300 bp fragment of Nicotiana benthamiana Phytoene Desaturase (PDS) (CDS nucleotides 580˜879; SEQ ID NO: 11) was used (deposited with the National Center for Biotechnology Information (NCBI) GenBank and having the accession number DQ469932.1 (accepted Dec. 4, 2007)). See Spitzer et al., Reverse genetics of floral scent: Application of tobacco rattle virus-based gene silencing in petunia, Plant Physiology 145: 1241-1250 (2007). The N. benthamiana PDS fragment shared 94.3% sequence identity with P. axillaris PDS gene.

Next, a 301 bp fragment of PhBSα (CDS nucleotides 3˜303 of SEQ ID NO: 3) and a 301 bp fragment of PhBSβ6 (CDS nucleotides 3˜303 of SEQ ID NO: 4) were tandemly cloned downstream of the PDS fragment in a pTRV2-PDS plasmid using ClonExpress II One Step Cloning Kit (Vazyme Biotech Co., Piscataway, NJ), yielding the final construct pTRV2-PDS-PhBSαp.

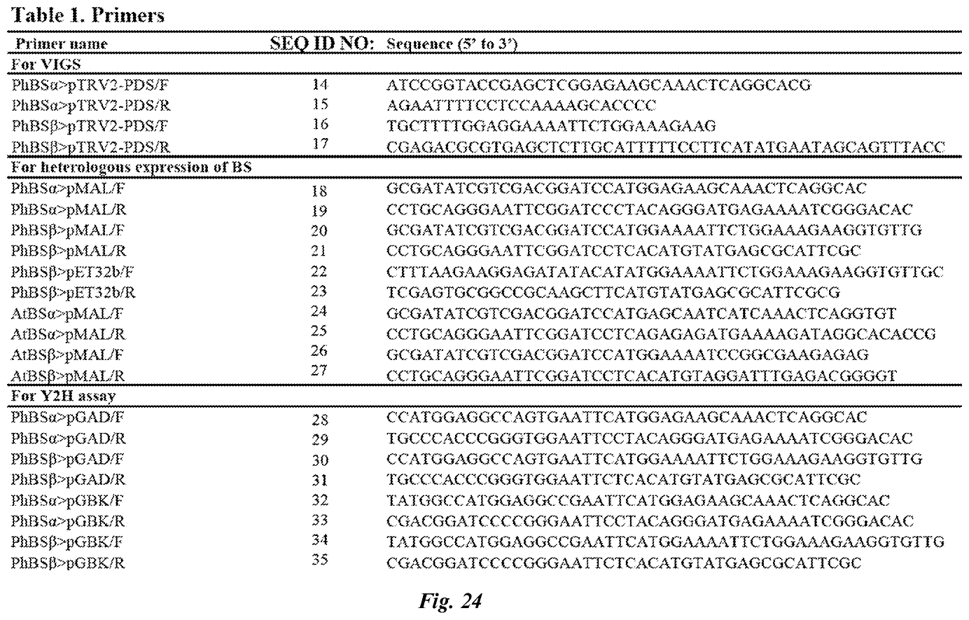

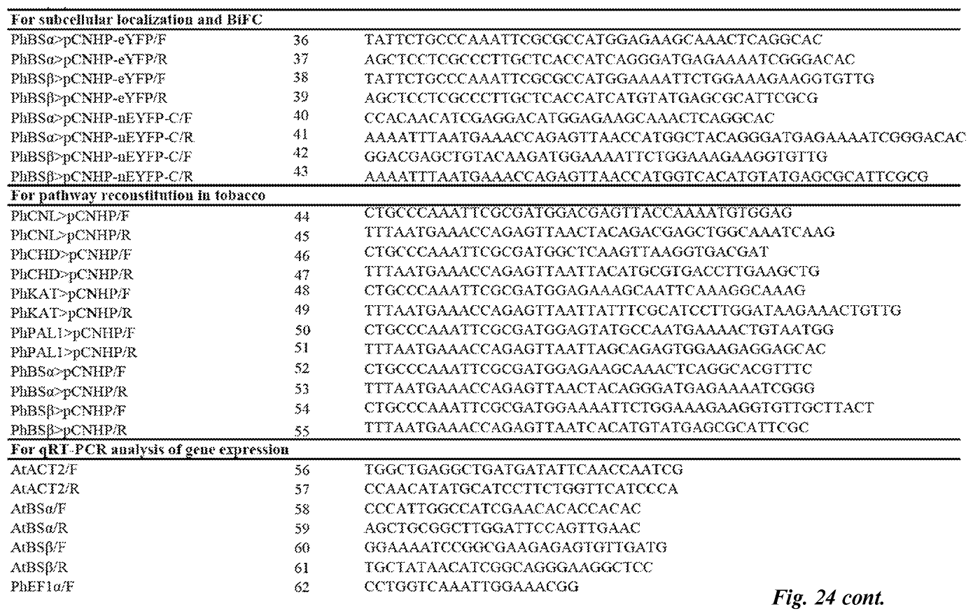

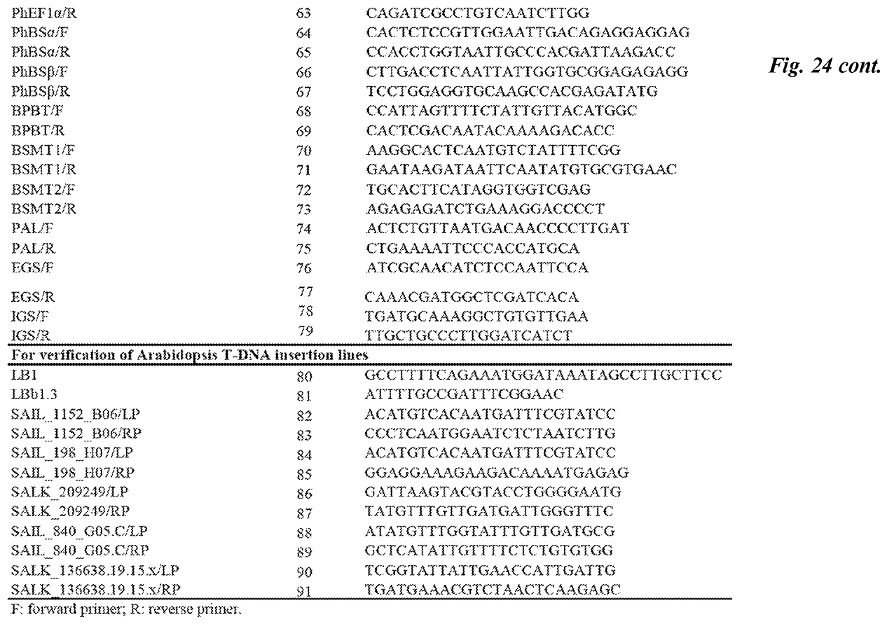

Before cloning, all fragments were verified to target only the desired genes and not to lead to off-target interference using the Sol Genomics Network VIGS Tool (publicly available online). The analysis was performed by comparing the PDS, PhBSα and PhBSβ6 VIGS target sequences against the P. axillaris and P. inflata (parental lines of P. hybrida) genomes using default parameters. See Bombarely et al., Insight into the evolution of the Solanaceae from the parental genomes of Petunia hybrida, Nature Plants 2: 16074 (2016). This analysis revealed that no other genes would be targeted by the PDS, PhBSα and PhBSβ double-stranded RNAs generated during the replication of viral genomes. PhBSα and PhBSβ fragments were amplified from P. hybrida petal cDNA via polymerase chain reaction (PCR) using gene-specific primers (see Table 1 in FIG. 24).

All binary vectors, including pTRV1, deposited with the NCBI GenBank and having accession number AF406990 (accepted Jun. 11, 2002), pTRV2-PDS and pTRV2-PDS-PhBSαp, were transformed into Agrobacterium tumefaciens strain GV3101. Single colonies for each construct were cultured at 28° C. in Luria-Bertani (LB) medium containing 50 mg/L rifampicin, 50 mg/L gentamycin, and 50 mg/L kanamycin to an optical density at a wavelength of 600 nm (OD₆₀₀) of 2.4.

Cells were pelleted, washed with infiltration buffer (50 mM MES, pH 5.7, 2 mM Na₃PO₄, 0.5% glucose and 200 μM acetosyringone), and incubated in the same buffer for 2 hours at room temperature. Yoo et al., An alternative pathway contributes to phenylalanine biosynthesis in plants via a cytosolic tyrosine:phenylpyruvate aminotransferase, Nature Communications 4: 2833 (2013).

Before infiltration, cultures containing pTRV1 and pTRV2 were mixed at a 1:1 ratio to reach a final OD₆₀₀ of 2.0. Using a blunt end syringe, the suspension was injected into the abaxial surface of all leaflets of 3- to 4-week-old petunia seedlings. Infiltrated seedlings were grown under normal greenhouse conditions until plant blooming. Plants infiltrated with pTRV1 and pTRV2-PDS served as negative controls.

The Arabidopsis T-DNA insertion lines were obtained from Arabidopsis Biological Resource Center (ABRC, Ohio State University, Columbus, OH) and propagated under normal growth room conditions. Genomic DNA was extracted from several individuals of each line. Homozygous individuals were identified using primers (Table 1 of FIG. 24) designed with SIGnAL (Salk Institute Genomic Analysis Laboratory, La Jolla, CA) a publicly accessible, online T-DNA Primer Design program.

### In Silico Analysis of Arabidopsis Genes

Tissue specific and developmental expression data of AtBSα and AtBSβ were obtained from the publicly available ePlant website. Fucile et al., ePlant and the 3D data display initiative: Integrative systems biology on the world wide web, PLoS One 6: e15237 (2011). Data for peptide enrichment of AtBSα and AtBSβ proteins in different tissues were retrieved from the publicly available Arabidopsis Peptide Atlas project website. van Wijk et al., The Arabidopsis PeptideAtlas: Harnessing worldwide proteomics data to create a comprehensive community proteomics resource, The Plant Cell (2021): doi:10.1093/plcell/koab211. Hierarchical clustering plot of AtBS genes as well as genes involved in β-oxidation and lignin biosynthesis pathways was generated using the publicly available ATTED-II Hcluster tool. Obayash et al., ATTED-II in 2018: A plant coexpression database based on investigation of the statistical property of the mutual rank index, Plant Cell Physiology 59: 440 (2018).

### Data Availability

The data supporting the findings hereof are available within Huang et al., A peroxisomal heterodimeric enzyme is involved in benzaldehyde synthesis in plants, Nature Communications 13: 1352 (2022) and its Supplementary Information files. The sequences reported in this paper have been deposited in NCBI GenBank database (all accepted Jan. 25, 2022) with the following accession numbers OK095279 for PhBSα (SEQ ID NO: 3), OK095280 for PhBSβ (SEQ ID NO: 4), OK095281 for AtBSα (SEQ ID NO: 101), OK095282 for AtBSβ (SEQ ID NO: 102), OK095283 for PdBSα (SEQ ID NO: 103), OK095284 for PdBSβ (SEQ ID NO: 104), OK095285 for SlBSα (SEQ ID NO: 105), and OK095286 for SlBSβ (SEQ ID NO: 106). Proteomic raw data and MaxQuant search results have been made publicly available through MassIVE with submission ID: MSV000088175, the entirety of which is incorporated by reference herein.

### Example 1

### Benzaldehyde Synthesis Via the β-Oxidation Pathway in Petunia

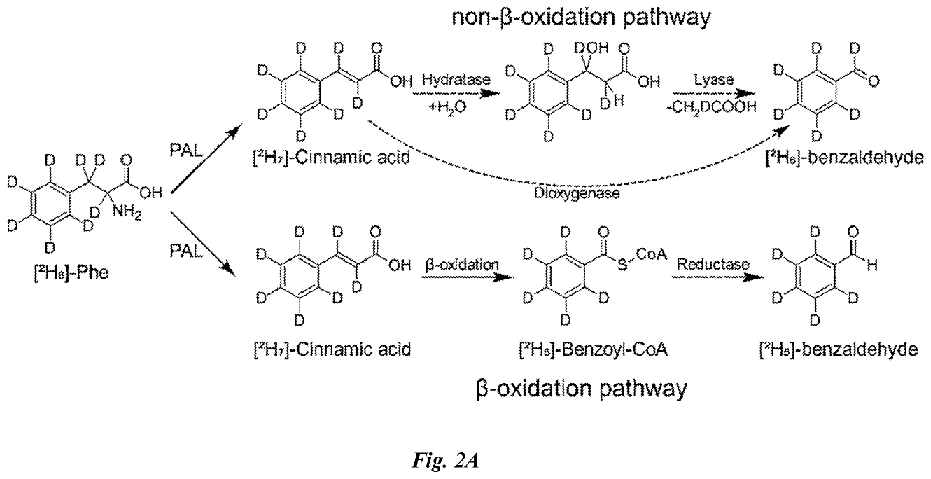

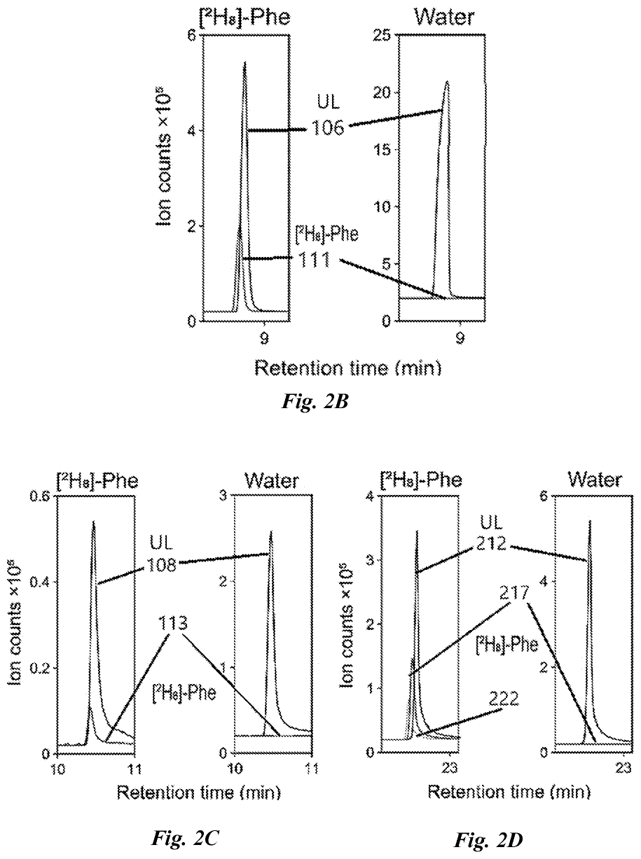

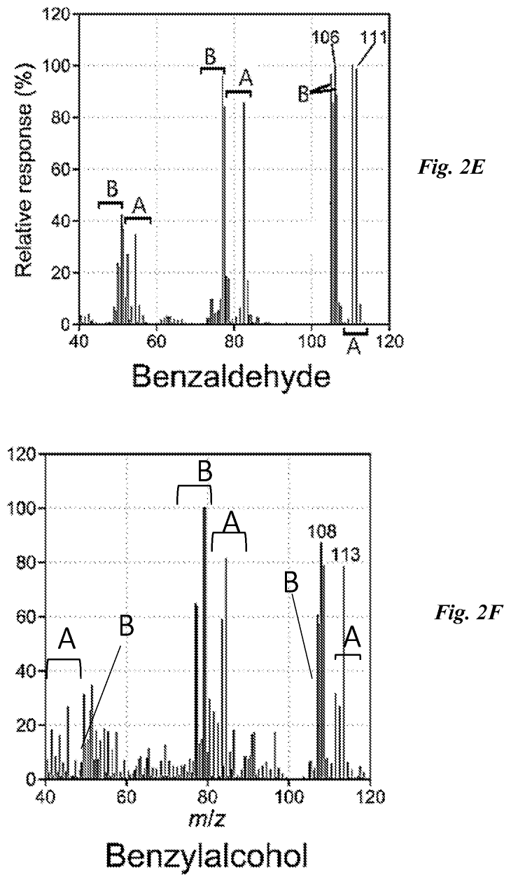

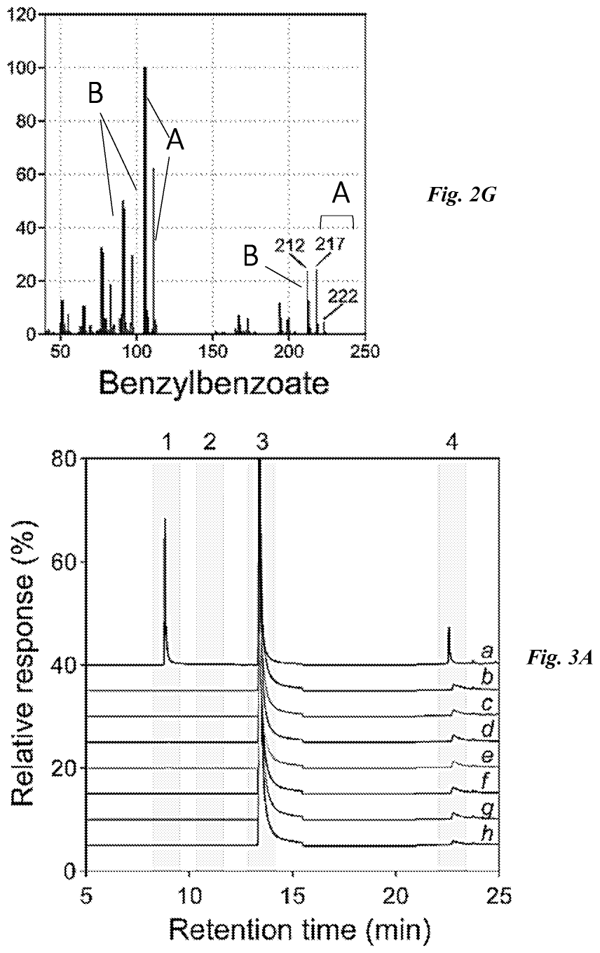

Two possible routes for biosynthesis of benzaldehyde and its predicted labeling from [2H₈]-Phe are shown in FIG. 2A, with established biochemical reactions presented by solid arrows and unidentified steps shown by dashed arrows. To determine whether benzaldehyde biosynthesis occurs via the β-oxidative or non-β-oxidative pathway, feeding experiments were performed as described in Boatright et al. (2004), supra.

Briefly, deuterium labeled L-phenylalanine (²H₈, 98%) and benzoic acid (ring-²H₈, 98%) were purchased from Cambridge Isotope Laboratories, Inc. (Andover, MA) and 50 mM [2H₈]-Phe or 100 mM [2H₈]-benzoic acid (neutralized with sodium hydroxide) were fed to excised limbs of 2-day-old petunia flowers for 2 hours, followed by the collection of volatiles for 4 hours from 18:00 to 22:00 (i.e., the time of the day with the highest scent emission).

Petunia flower volatiles were collected by a closed-loop stripping method and analyzed by gas chromatography mass spectrometry (GC-MS) as described in Qian et al., Completion of the cytosolic post-chorismate phenylalanine biosynthetic pathway in plants, Nature Communications 10: 15 (2019), with minor modifications. More specifically, volatiles were collected during the indicated hours from a minimum of three 2-day-old flowers per biological replicate. Absorbed volatiles were eluted from collection traps containing 20 mg of Porapak Q (80-100 mesh) (Waters, Milford, MA) with 200 μl dichloromethane (DCM) supplemented with 1 μg of naphthalene as the internal standard. For the analysis of internal pools, 100 mg of ground tissue was extracted twice at 4° C. with 500 μL DCM containing 1 μg naphthalene as the internal standard. The extracts were pooled together and concentrated to about 200 μL under a mild stream of nitrogen gas, and samples were analyzed on an Agilent 7890B-5977B gas chromatography mass spectrometry (GC-MS) system (Agilent Technologies, Inc., Santa Clara, CA) using an analytical method translated from an established method with the Agilent 6890N-5975B GC-MS system as previously described. Qian et al. (2019), supra.

FIG. 2B shows the results of the GC-MS analysis of benzaldehyde, FIG. 2C shows the results of the GC-MS analysis of benzyalcohol, and FIG. 2D shows the results of the GC-MS analysis of benzylbenzoate (indicated in m z). Unlabled compounds in each graph are identified with “UL” and [²H₈]-Phe labeled compounds are identified with “[²H₈]-Phe”.

Quantification of different volatile organic compounds (VOCs) was then performed based on standard curves generated with authentic standards, with the results shown in Table 2. The values shown in Table 2 are percentage of labeling, means±SE, and n=3 biological replicates (n.d., not detected). The data were calculated based on peak areas integrated from extracted ion current (EIC) chromatograms of labeled (number of additional mass units, m/z used for calculation, as indicated in the brackets) and unlabeled compounds.

TABLE 2
Labeling of VOCs after feeding of deuterium
labeled phenylalanine and benzoic acid

| | ²H₈- | ²H₅-Benzoic |
|---|---|---|
| Labeled compounds | Phenylalanine (%) | acid (%) |
| Benzaldehyde (D5, 111) | 22.51 ± 5.45 | 23.03 ± 2.20 |
| Benzylalcohol (D5, 113) | 12.87 ± 3.36 | 12.37 ± 1.03 |
| Phenylacetaldehyde (D7 + | 29.76 ± 8.26 | n.d. |
| D8, 97 + 98) | | |
| Methylbenzoate (D5, 110) | 25.69 ± 4.65 | 71.45 ± 3.44 |
| Phenylethanol (D7, 98) | 22.32 ± 7.34 | n.d. |
| Eugenol (D5, 169) | 5.79 ± 1.84 | n.d. |
| Vanillin (D4, 156) | 4.41 ± 1.05 | n.d. |
| Isoeugenol (D5, 169) | 4.94 ± 0.94 | n.d. |
| Benzylbenzoate (D5, 217) | 11.92 ± 1.90 | 24.33 ± 4.09 |
| Benzylbenzoate (D10, 222) | 2.88 ± 0.64 | 9.97 ± 3.53 |
| Benzoyl moiety of | 7.76 ± 1.12 | 22.88 ± 5.54 |
| Benzylbenzoate (105) | | |
| Alcohol moiety of | 10.80 ± 2.31 | 21.86 ± 4.98 |
| Benzylbenzoate (91) | | |

If formation of benzaldehyde occurred via the non-β-oxidative route, the label at the aldehyde position of the product would be expected to result in benzaldehyde molecules+6 atomic mass units larger (FIG. 2A). However, benzaldehyde was labeled by +5 atomic mass units, supporting that its biosynthesis instead proceeds via the β-oxidative pathway (FIGS. 2A and 2E and Table 2). Similarly, benzylalcohol was labeled by +5 atomic mass units (FIGS. 2A and 2E and Table 2) indicating that benzylalcohol also originates from the β-oxidative pathway. These findings support the interconversion between benzylalcohol and benzaldehyde. Boatright et al. (2004), supra.

The lower labeling of benzylalcohol relative to benzaldehyde was due to its dilution by the large internal pool of unlabeled benzylalcohol that exists in petunia flowers. Liao et al., Cuticle thickness affects dynamics of volatile emission from petunia flowers, Nature Chemical Biology 17: 138-145 (2021). Indeed, both benzoyl-CoA and benzylalcohol moieties were labeled by +5 atomic mass units in benzylbenzoate (FIGS. 2D and 2G and Table 2).

To further examine precursor-product relationships and because the in vivo labeling experiments with petunia petals supported that benzoyl-CoA serves as a benzaldehyde precursor, it was tested whether benzoyl-CoA reductase activity in planta was present.

First, ²H₈-benzoic acid was supplied to excised petunia corollas for 2 hours and floral volatiles were analyzed for their isotopic abundances. Benzoic acid can be activated to benzoyl-CoA by 4-coumarate:CoA ligase-like enzymes, thus providing potential precursors for benzaldehyde and subsequently benzylalcohol syntheses. Both benzaldehyde and benzylalcohol were labeled in these experiments along with benzylbenzoate (Table 2), which relies on benzoyl-CoA derived predominantly from the β-oxidative pathway. Adebesin et al., A peroxisomal thioesterase plays auxiliary roles in plant β-oxidative benzoic acid metabolism, The Plant J. 93: 905-916 (2018).

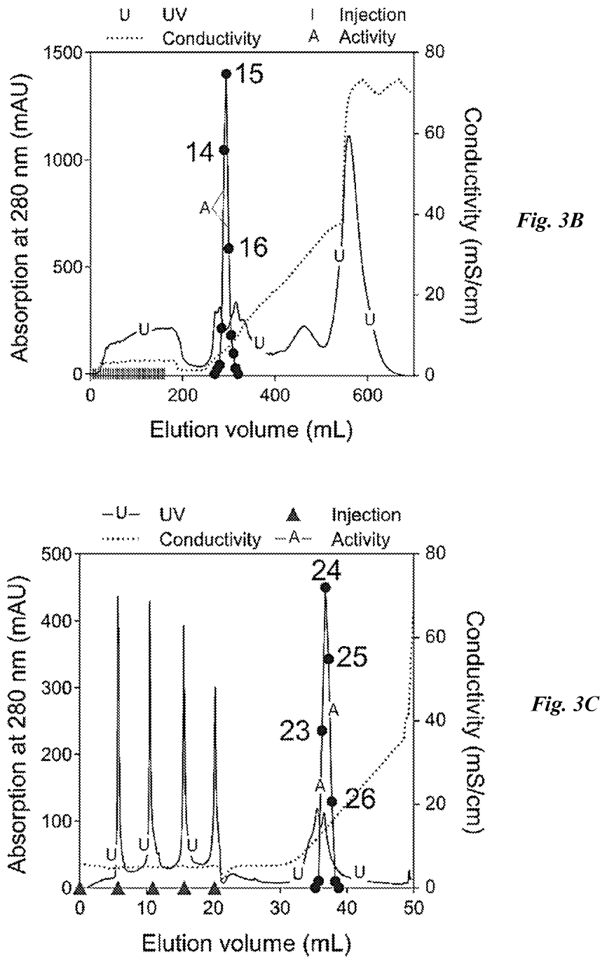

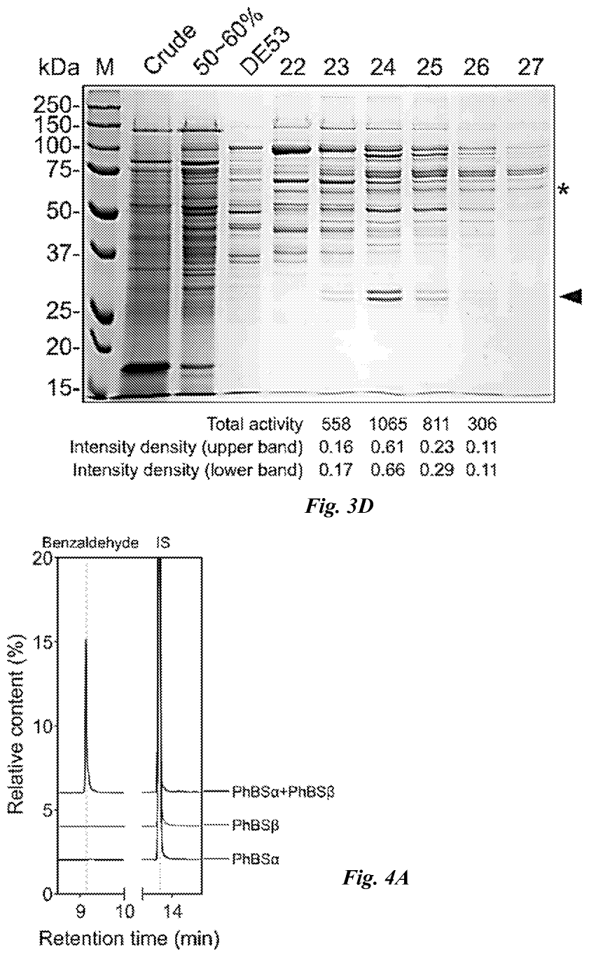

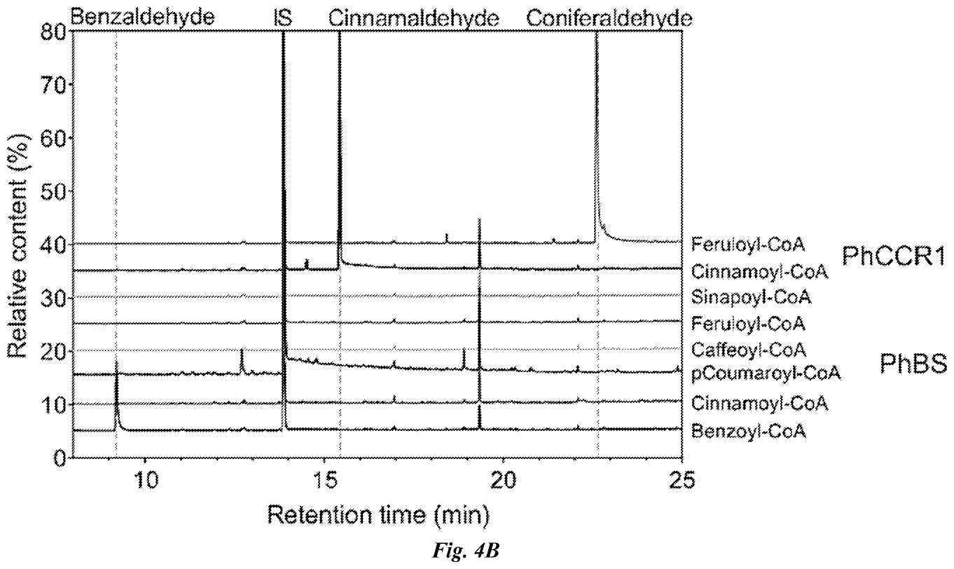

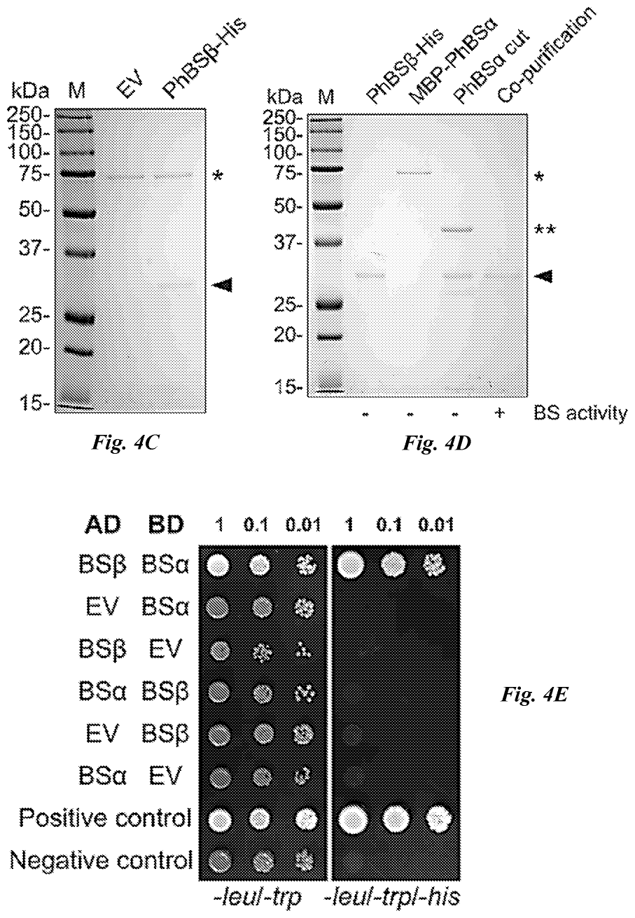

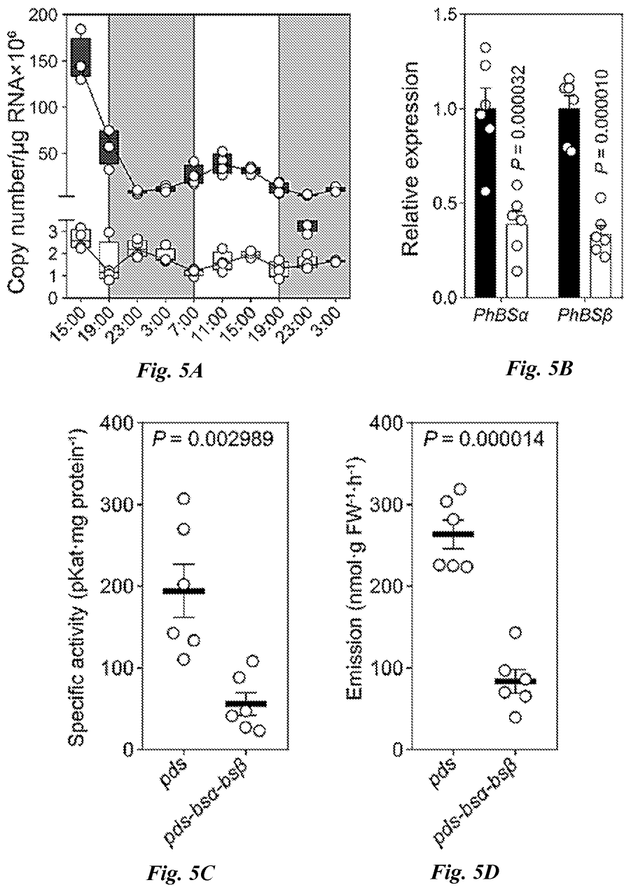

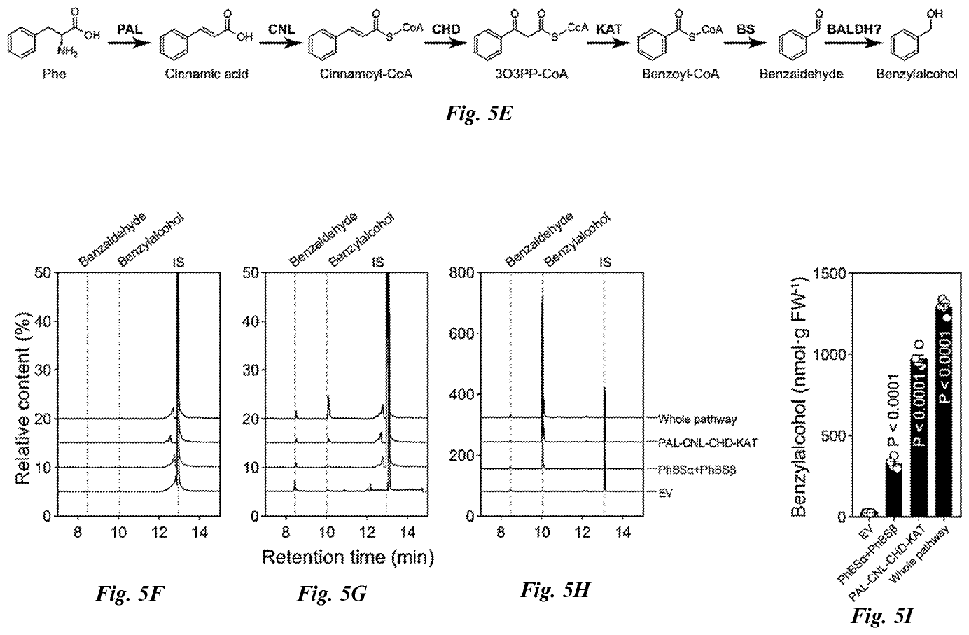

Crude protein extracts from petunia petals collected around the peak of emission were then incubated with several different assay mixtures containing benzoyl-CoA and different reducing cofactors; namely, (a) crude extracts+benzoyl-CoA+NADPH; (b) crude extracts+benzoyl-CoA+NADP⁺; (c) crude extracts+benzoyl-CoA+NADH; (d) crude extracts+benzoyl-CoA+NAD⁺; (e) crude extracts+benzoyl-CoA; (f) crude extracts+benzoic acid+NADPH; (g) heated denatured crude extracts+benzoyl-CoA+NADPH; and (h) crude extracts+cinnamic acid. The crude protein extracts were obtained by extraction of petunia petal tissue with buffer A (3:1 [v/w] buffer/tissue). After centrifugation of slurry at 15,000 g for 20 minutes, the supernatant was desalted with Econo-Pac 10DG Columns (Bio-Rad, Hercules, CA) according to the manufacturer's protocol and 20 μL of desalted protein was used in the activity assays. The reaction was performed for 30 minutes at 28° C. and the product was extracted with 200 μL DCM containing 1 μg naphthalene (internal standard). After centrifugation at 15,000 g for 20 minutes, 2 μL of the bottom DCM phase was injected in GC-MS, with results shown in FIG. 3A. The compounds labeled in FIG. 3A with numbers are (1) benzaldehyde, (2) benzylalcohol, (3) internal standard (naphthalene), and (4) benzylbenzoate and the different assay mixtures described above are identified with their above-associated letters.

Efficient conversion of benzoyl-CoA to benzaldehyde was observed only in the presence of NADPH (a of FIG. 3A). No benzaldehyde was formed when benzoyl-CoA was replaced by benzoic acid or cinnamic acid or when NADH, NADP⁺, NAD⁺ were supplied instead of NADPH in reaction with benzoyl-CoA (b-h of FIG. 3A). Controls lacking NADPH or containing denatured crude extract produced no detectable product (e, g, and h of FIG. 3A).

Since petunia petals contain a large internal pool of benzylbenzoate, this compound was detected in all reactions despite using desalted proteins in assays. However, its level increased only upon incubation of crude protein extracts with benzoyl-CoA and NADPH, supporting that part of the produced benzaldehyde was reduced to benzylalcohol, which was then rapidly converted to benzylbenzoate in the presence of excessive benzoyl-CoA amount through the action of benzoyl-CoA:benzyl alcohol/2-phenylethanol benzoyltransferase (BPBT). Boatright et al. (2004), supra; Orlova et al., Reduction of benzenoid synthesis in petunia flowers reveals multiple pathways to benzoic acid and enhancement in auxin transport, The Plant Cell 18: 3458-3475 (2006).

Taken together, these results support that benzaldehyde is synthesized via the β-oxidative pathway and a putative benzaldehyde synthase catalyzes the NADPH-dependent reduction of benzoyl-CoA to the corresponding aldehyde.

### Example 2

### Partial Purification and Identification of Benzaldehyde Synthase

The petunia genome contains two cinnamoyl-CoA reductase (CCR) genes, out of which PhCCR1 is highly expressed in scent-emitting flower tissues. Muhlemann et al., The monolignol pathway contributes to the biosynthesis of volatile phenylpropenes in flowers, New Phytologist 204: 661-670 (2014). Biochemical characterization of the corresponding enzyme revealed that it is most active with feruloyl-CoA, followed by sinapoyl-CoA and p-coumaroyl CoA, and possesses a very low activity with caffeoyl-CoA and benzoyl-CoA. Pan et al., Structural studies of cinnamoyl-CoA reductase and cinnamyl-alcohol dehydrogenase, key enzymes of monolignol biosynthesis, The Plant Cell 26: 3709-3727 (2014). Moreover, the lack of changes in the benzoyl-CoA pool size and benzaldehyde emission upon 90% RNA interference (RNAi) suppression of PhCCR1 expression indicates that efficient benzaldehyde formation by petunia petals and detected benzaldehyde synthase (BS) activity (FIG. 3A) respectively relies on and belongs to an enzyme other than PhCCR1. Muhlemann et al. (2014), supra.

To test this, BS was partially purified using ammonium sulfate precipitation, DE53 anion exchange (FIG. 3B) and Mono Q chromatography (FIG. 3C). Briefly, petunia flower limbs were collected from 9 PM to 10 PM, flash frozen in liquid nitrogen and stored at −80° C. until protein purification. All purification procedures were performed on ice or at 4° C. except as noted. In a typical purification, 60 grams of petal tissue were ground in liquid nitrogen to a fine powder using a mortar and pestle. 240 mL of protein extraction buffer A (100 mM Tris, pH 7.4, 150 mM NaCl, 1 mM ethylenediaminetetraacetic acid, 1% (v/v) Triton X-100, 10% (v/v) glycerol, 10 mM dithiothreitol, and 1 mM phenylmethanesulfonyl fluoride) were immediately added to the powder.

The slurry was ground for additional 10 minutes with a pestle and incubated on ice for 30 minutes with frequent mixing. After centrifugation at 10,000 g for 30 minutes, the supernatant was passed through double layer of Miracloth (Calbiochem Research Biochemicals, St. Louis, MO) and ammonium sulfate was added sequentially in a 10% increment starting from 40% saturation up to 80% saturation.

After 20 minutes of gentle rotation at each step, the ammonium sulfate precipitation solution was centrifuged at 12,000 g for 10 minutes. Protein pellets from each precipitation step were re-suspended in 24 mL of buffer B (20 mM Tris, pH 7.4, 10% (v/v) glycerol).

The fraction of 50˜60% ammonium sulfate saturation was used for further purification. Briefly, the fraction was diluted to 144 mL with buffer B, passed through a 0.45 μm filter and loaded onto a diethylaminoethyl (DEAE)-cellulose anion exchange column (25×65 mm column containing 10 grams DE53; Whatman plc, Maidstone, UK) at the flow rate of 2 mL min⁻¹ using a fast protein liquid chromatography (FPLC) system (AKTA, GE Healthcare, Chicago, IL). After the unabsorbed material was washed with 60 mL of buffer B, proteins were eluted from the column with a linear gradient (300 mL) from 0 to 500 mM NaCl in buffer B. FIG. 3B shows BS activity in the petunia crude extracts, with the fractions with BS activity numbered (i.e., fractions 14, 15, and 16).

Fractions of 5 mL were collected and assayed for BS activity.

BS activity assays of the fractions were carried out in 100 μL reaction mixture containing 50 mM Bis-Tris buffer, pH 6.5, 200 μM benzoyl-CoA, and 2 mM freshly prepared NADPH. The reaction was initiated by adding a protein and incubated at 28° C. for 30 minutes, and the reactions were then terminated by adding 100 μL of 100% ice-cold methanol (MeOH). The BS enzyme assays were performed at an appropriate enzyme concentration so that reaction velocity was proportional to enzyme concentration and linear during the incubation time period. Kₘ and Vₘₐₓ were determined by non-linear fit to the Michaelis-Menten equation using Graphpad Prism, v8.2.0.

Triplicate assays were performed for all data points.

After centrifugation at 15,000 g for 20 minutes, 5 μL of the supernatant were subjected to high-performance liquid chromatography (HPLC) analysis using an Agilent 1260 Infinity II system (Agilent Technologies, Inc., Santa Clara, CA) equipped with an InfinityLab Poroshell 120 EC-C18 column (3.0×150 mm, 2.7 μm) maintained at 35° C. Products were separated with a 15-minute linear gradient of 10% aqueous acetonitrile solution to 100% acetonitrile and monitored at 248 nm using a diode array detector (DAD). Benzaldehyde was identified by comparing the retention time and absorption spectrum with authentic standard.

Quantitation was achieved by using standard curve generated from authentic standard and product verification was performed by GC-MS. Namely, crude protein extracts were obtained by extraction of petunia petal tissue with buffer A (3:1 [v/w] buffer/tissue). After centrifugation of slurry at 15,000 g for 20 minutes, the supernatant was desalted with Econo-Pac 10DG Columns (Bio-Rad Laboratories, Inc., Hercules, CA) according to the manufacturer's protocol and 20 μL of desalted protein was used in BS activity assays. The reaction was performed for 30 minutes at 28° C. and the product was extracted with 200 μL DCM containing 1 μg naphthalene (internal standard). After centrifugation at 15,000 g for 20 minutes, 2 μL of the bottom DCM phase was injected in GC-MS.

Fractions 14 and 15 with the highest BS activity eluted at about 125 mM NaCl were pooled, diluted to 15 mL with buffer B and loaded at the flow rate of 0.5 mL min⁻¹ onto anion exchange column MonoQ™ 5/50 GL (GE Healthcare, Chicago, IL) pre-equilibrated with buffer B. The column was washed with 5 mL of buffer B and the bound protein was eluted using a linear gradient (20 mL) from 0 to 500 mM NaCl in buffer B. Fractions of 0.5 mL were collected and analyzed for BS activity.

Fraction SEC 24, with the highest BS activity eluted at about 170 mM NaCl, was further subjected to size-exclusion gel filtration chromatography on a Superose 12 10/300 GL column (GE Healthcare, Chicago, IL) using buffer C for column equilibration and elution at the flow rate of 0.4 mL min-1. Fractions of 0.5 mL were collected and analyzed for BS activity.

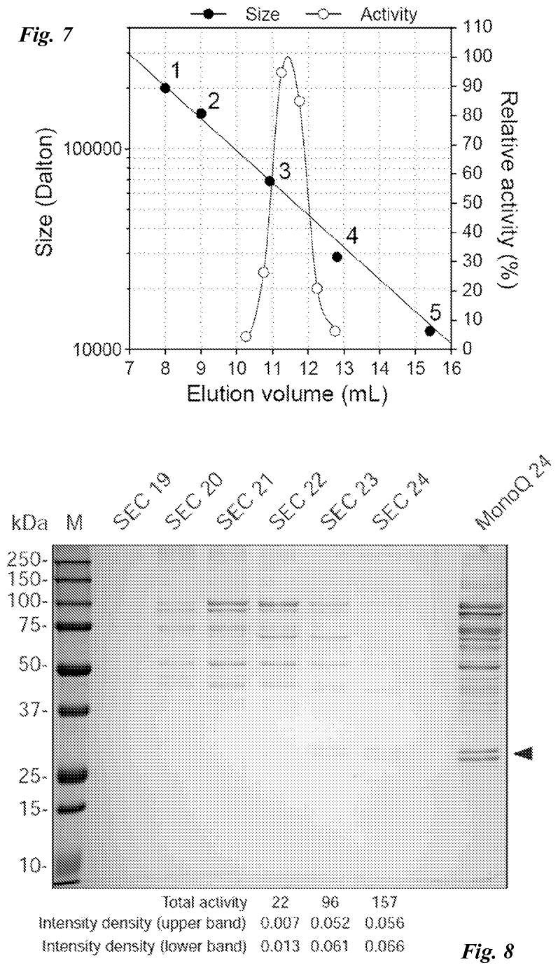

In addition to the foregoing, to determine the apparent molecular weight of native PhBS, a small fraction (200 μL) of crude petunia flower protein extract was loaded onto a Superose 12 10/300 size exclusion column and eluted with buffer C (20 mM Tris, pH 7.4, 150 mL NaCl). Fractions of 0.5 mL were also assayed for BS activity which is plotted against elution volume (curved line) in FIG. 7. The column was calibrated with the following standard markers from Gel Filtration Markers Kit (Millipore Sigma, St. Louis, MO): (1) β-amylase (200 kDa), (2) alcohol dehydrogenase (150 kDa), (3) bovine serum albumin (66 kDa), (4) carbonic anhydrase (29 kDa), and (5) cytochrome c (12.4 kDa), with their elution behavior shown with the straight diagonal line in FIG. 7. Based on these results, native BS appeared to be 58 kDa (FIG. 7).

Fractions from all purification steps were precipitated with acetone, re-dissolved in 10 μL of loading buffer, and analyzed with 12% sodium dodecyl-sulfate polyacrylamide gel electrophoresis (SDS-PAGE) analysis followed by gel staining with Coomassie brilliant blue (CBB) R-250. FIG. 3D shows the results of an SDS-PAGE analysis, with the indicated lanes corresponding to crude; petunia flower crude protein extract (˜40 μg); 50-60%, proteins precipitated at 50-60% ammonium sulfate saturation (˜40 μg); DE53, combined fractions 14 and 15 after DE53 chromatography (˜20 μg); and 22-27, fractions separated by MonoQ chromatography shown in FIG. 3C (10 μL each). The asterisk (*) in FIG. 3D indicates the apparent size of native PhBS (around 60 kDa), and the triangle indicates the position of two closely migrated bands representing PhBSα and PhBSβ.

FIG. 8 shows the SDS-PAGE analysis results for fractions SEC 19 to SEC 24 from the size exclusion chromatography study of FIG. 7 as well as 10 μL of the MonoQ fraction 24 (loaded into the far-right lane of the gel). Total BS activity (pKat·mg protein⁻¹) and intensity density of BS subunits for fractions SEC 22-SEC 24 are shown below the gel in FIG. 8 and the triangle indicates the position of the BS subunits.

Finally, fractions 24, 25 and 26 from MonoQ™ chromatography (MonoQ 24 to 26) and one fraction from gel filtration chromatography of the MonoQ fraction 24 containing BS activity were also subjected to proteomic analysis at Purdue Proteomics Facility (Bindley Bioscience Center, Purdue University, West Lafayette, IN) as described in Qian et al. (2019), supra.

This purification protocol resulted in up to 80.5-fold increase in specific activity over the crude extract, with a recovery of 3.8% (see Table 3 below for purification data; sum of recovery percentages of MonoQ-23 through MonoQ-26).

TABLE 3
PhBS purification from petunia petals.

| | Total | Protein | Specific | Purifi- | |
|---|---|---|---|---|---|
| | activity | content | activity | cation | Recovery |
| Fraction | (pKat) | (mg) | (pKat/mg) | fold | (%) |
| Crude extract | 72,533 | 1094.4 | 66.3 | 1.0 | 100.0 |
| (60 g tissue) | | | | | |
| Ammonium | 23,680 | 199.7 | 118.6 | 1.8 | 32.7 |
| sulfate (50-60%) | | | | | |
| DE53-14 | 3167 | 2.33 | 1359 | 20.5 | 4.4 |
| DE53-15 | 4246 | 2.38 | 1788 | 27.0 | 5.8 |
| MonoQ-23 | 558 | 0.203 | 2748 | 41.5 | 0.8 |
| MonoQ-24 | 1065 | 0.232 | 4589 | 69.2 | 1.5 |
| MonoQ-25 | 811 | 0.152 | 5336 | 80.5 | 1.1 |
| MonoQ-26 | 306 | 0.086 | 3559 | 53.7 | 0.4 |

Ultimately, four fractions with specific activities ranging from 2748 pKat·mg protein⁻¹ to 5336 pKat·mg protein⁻¹ were obtained after MonoQ anion-exchange chromatography (Table 3). However, when the four MonoQ fractions with the highest specific activities were analyzed on SDS-PAGE, no proteins with size between 50 kDa and 75 kDa displayed a positive correlation with BS activities in the various fractions; instead, intensities of two closely migrating bands around 30 kDa well correlated with BS activities (FIG. 3D; see arrow). The sum of molecular masses of these two bands calculated from their migration in SDS-PAGE gel was close to that of native plant BS as determined by gel filtration chromatography. Moreover, gel filtration chromatography of the MonoQ fraction 24, which contained the highest amounts of the two proteins, also revealed a strong positive correlation between the presence of the two bands and BS activities (FIG. 8), suggesting that petunia BS is a homodimer of either of the individual proteins or composed of two distinct subunits.

Peptides obtained by ultra-performance liquid chromatography quadrupole time of flight mass spectrometry (UPLC-QTOF MS/MS) were searched against the protein sequences encoded by Petunia axillaris genome (downloaded from Sol Genomics Network draft genome sequence v1.6.2 (publicly available)) with NAD(P)-binding proteins being of a particular interest. A total of 744 proteins were identified from all analyzed fractions, with only Peaxi162Scf00811g00011 (SEQ ID NO: 12) and Peaxi162Scf00776g00122 (SEQ ID NO: 13) encoding proteins around 30 kDa in fraction SEC 24. Since fraction SEC 24 contained no other NAD(P)-binding proteins despite the highest b activity among all gel filtration fractions, this supported one or both of these two proteins are responsible for the BS activity. Therefore, based on results presented below, the slightly larger protein encoded by Peaxi162Scf00811g00011 (SEQ ID NO: 12) was designated a Petunia hybrida alpha subunit of benzaldehyde synthase (PhBSα), and the smaller protein encoded by Peaxi162Scf00776g00122 (SEQ ID NO: 13) was designated a beta subunit of benzaldehyde synthase (PhBSβ).

PhBSα and PhBSβ6 encode proteins of 30.5 kDa and 29.6 kDa, respectively, which belong to NAD(P)-binding Rossmann-fold superfamily and exhibit 30.3% identity and 50.4% similarity to each other. Accordingly, phylogenetic analysis of three petunia species, Petunia hybrida and its two parental lines Petunia axillaris and Petunia inflata was performed. The evolutionary history was inferred by using the Maximum Likelihood method based on the Jones-Taylor-Thornton (JTT) matrix-based model. Maximum-likelihood phylogenetic tree was constructed using MEGA7 with the suitable model inferred by “Find Best DNA/Protein Models (ML)” algorithm. Phylogenetic trees were tested with Bootstrap method for 500 replications.

The tree with the highest log likelihood (˜1988.16) is shown FIG. 9. The percentage of trees in which the associated taxa clustered together is shown next to the branches. Initial tree(s) for the heuristic search were obtained automatically by applying Neighbor-Join and BioNJ algorithms to a matrix of pairwise distances estimated using a JTT model, and then selecting the topology with superior log likelihood value. A discrete Gamma distribution was used to model evolutionary rate differences among sites (8 categories (+G, parameter=1.3343)). The rate variation model allowed for some sites to be evolutionarily invariable ([+1], 8.33% sites)). The phylogenetic analysis revealed that only a single gene exists in the Petunia genus for the 0-subunit, while the α-subunit gene has more homologs (FIG. 9).

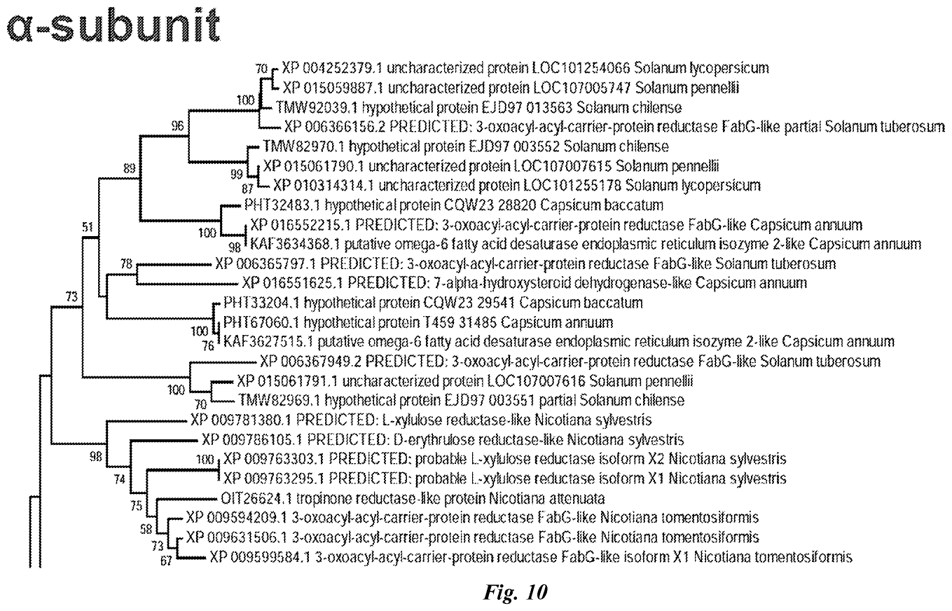

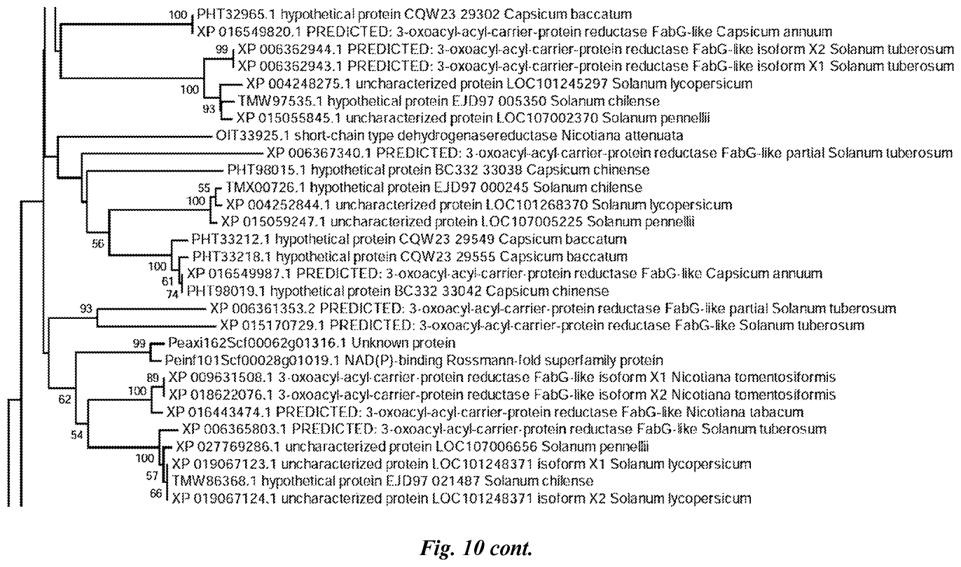

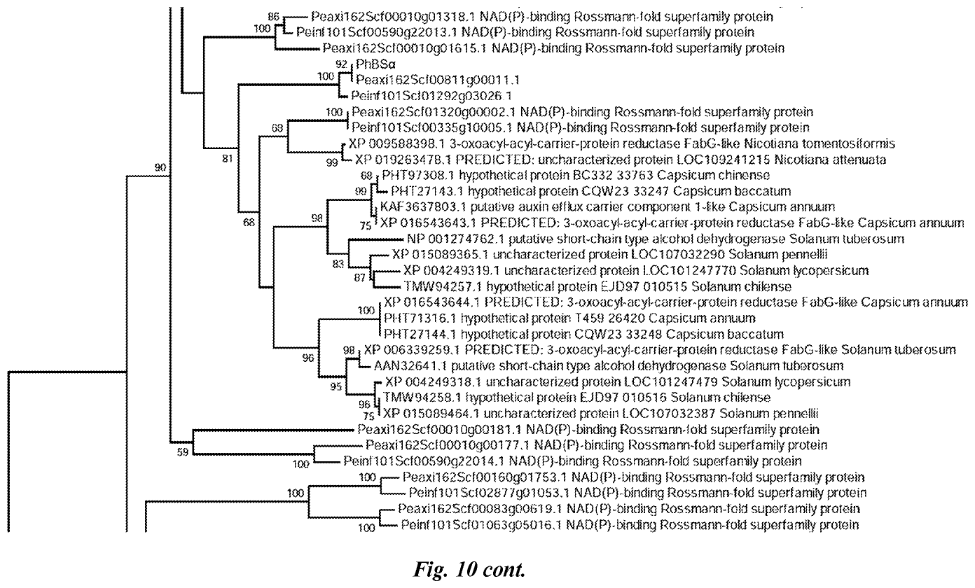

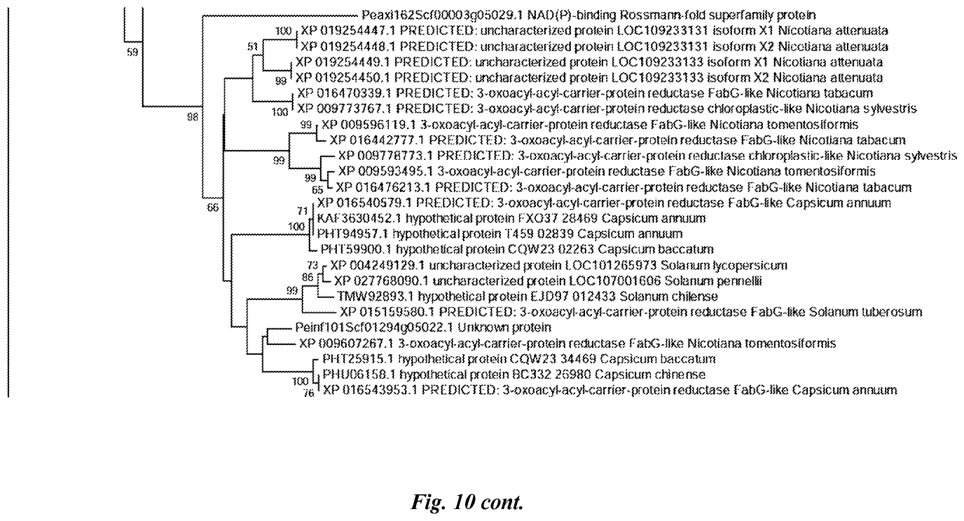

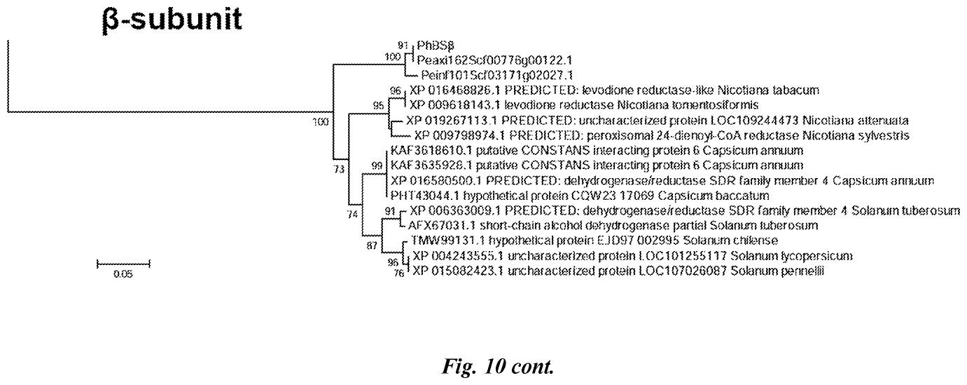

Thereafter, the phylogenetic analysis was extended to the whole Solanaceae family, with the evolutionary history inferred using the Neighbor-Joining method. Namely, the Neighbor-Joining algorithm in MEGA7 program was used to build the phylogenetic tree shown on FIG. 10. The phylogenetic trees were tested with Bootstrap method for 1000 replications (Neighbor-Joining tree).

The optimal tree with the sum of branch length equals 6.0 is shown in FIG. 10. The percentage of replicate trees in which the associated taxa clustered together in the bootstrap test (1000 replicates) are shown next to the branches. In the Solanaceae family, the α-subunit appeared to reside in a large clade containing 3-oxoacyl-(acyl-carrier-protein) reductase FabG-like proteins that participate in fatty acid biosynthesis, while the β-subunit is a member of a more limited clade with unknown functions (FIG. 10). Pursuant to the phylogenetic analysis results, the sequences related to the identified proteins in FIG. 10 are homologs for SEQ ID NOS: 1 and 2, as applicable.

### Example 3

### Benzaldehyde Synthase Uses Benzoyl-CoA to Produce Benzaldehyde In Vitro

To determine the possible function of the proteins encoded by PhBSα and PhBSβ, the coding regions of both subunits were amplified from petunia petal cDNAs, subcloned into expression vectors, and expressed in Escherichia coli (see Materials section above for protocol details).

Recombinant BS proteins were produced in E. coli by expressing the corresponding coding regions (CDS) subcloned into an expression vector pMAL-c5X (New England BioLabs, Ipswich, MA) containing a maltose binding protein (MBP) tag using ClonExpress II One Step Cloning Kit (Vazyme Biotech Co., Piscataway, NJ).

Both Petunia and Arabidopsis CDSs were PCR amplified from the corresponding flower cDNAs with gene-specific primers (see Table 1 of FIG. 24), while almond (PdBSα: deposited with the NCBI GenBank and having accession number XP_034223352 (accepted May 14, 2020) (SEQ ID NO: 97); PdBSβ: deposited with NCBI GenBank and having accession number XP_034208025.1 (accepted May 14, 2020) (SEQ ID NO: 98) and tomato proteins (SlBSα: deposited with NCBI GenBank and having accession number XP_004249319.1 (accepted Aug. 7, 2018) (SEQ ID NO: 99); SlBSβ: deposited with NCBI GenBank and having accession number XP_004243555.1 (accepted Aug. 8, 2018) (SEQ ID NO: 100)) were identified by BLAST of amino acid sequences of PhBS subunits against the NCBI non-redundant protein database.

Candidates with the highest sequence identities were chosen, and their corresponding CDSs were codon-optimized and synthesized (GenScript Biotech Corp, Piscataway, NJ). After sequence verification, the desired plasmids were transformed into Rosetta2 (DE3) pLysS competent cells (MilliporeSigma, Burlington, MA). Single colonies were picked and cultured overnight in 2 ml LB medium containing 100 mg/L carbenicillin and 34 mg/L chloramphenicol at 37° C. Overnight cultures were then diluted 100-fold with LB medium and continued to culture at 37° C. until OD₆₀₀ reached ˜0.5.

After cooling the cell culture on ice for 10 minutes, isopropyl β-D-1-thiogalactopyranoside (IPTG) was added to a final concentration of 0.1 mM and cells were incubated at 28° C. with shaking for additional 2.5 to 3 hours. After harvesting cells by centrifugation, their lysis and protein purification were performed according to the manufacturer's protocol of Amylose Resin (New England Biolabs, Ipswich, MA).

For pull-down experiments, the PhBSβ CDS was amplified, cloned into the NdeI and HindIII sites of pET32b expression vector in-frame with a C-terminal 6×His-tag (Sigma-Aldrich, St. Louis, MO) using ClonExpress II One Step Cloning Kit (Vazyme Biotech Co., Piscataway, NJ), and the protein was expressed in E. coli. The MPB tag (˜42.5 kDa) was then removed from the purified MBP-PhBSα by digestion with Factor Xa protease (New England Biolabs, Ipswich, MA) overnight at 4° C., and PhBSα was mixed with bacterial cell lysate containing soluble PhBSβ-His₆. The reconstituted PhBS complex was purified using Ni-NTA agarose (Qiagen Sciences, Hilden, Germany) following the manufacturer's protocol and analyzed for BS activity. Alternatively, bacterial lysates containing MBP-PhBSα were mixed with PhBSβ-His₆ and the reconstituted PhBS complex was purified using amylose resin.

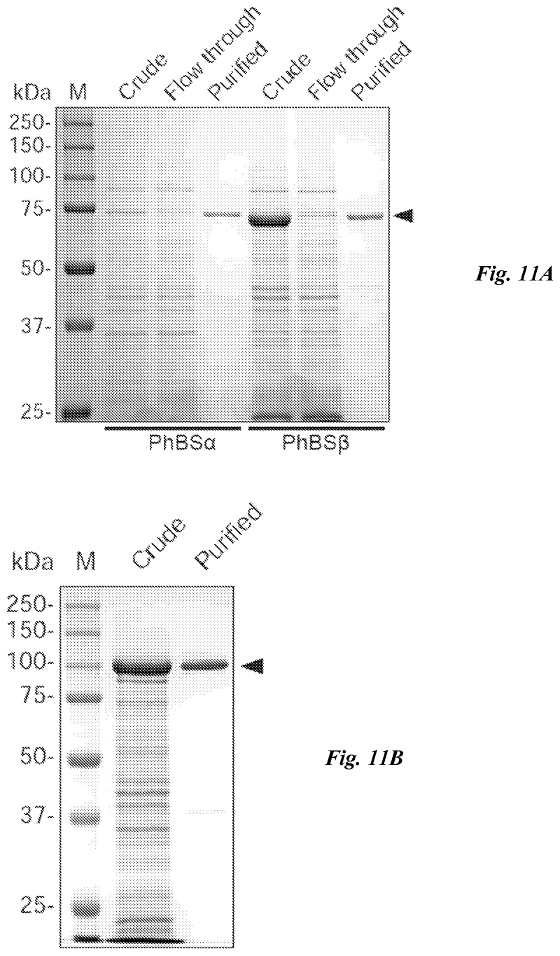

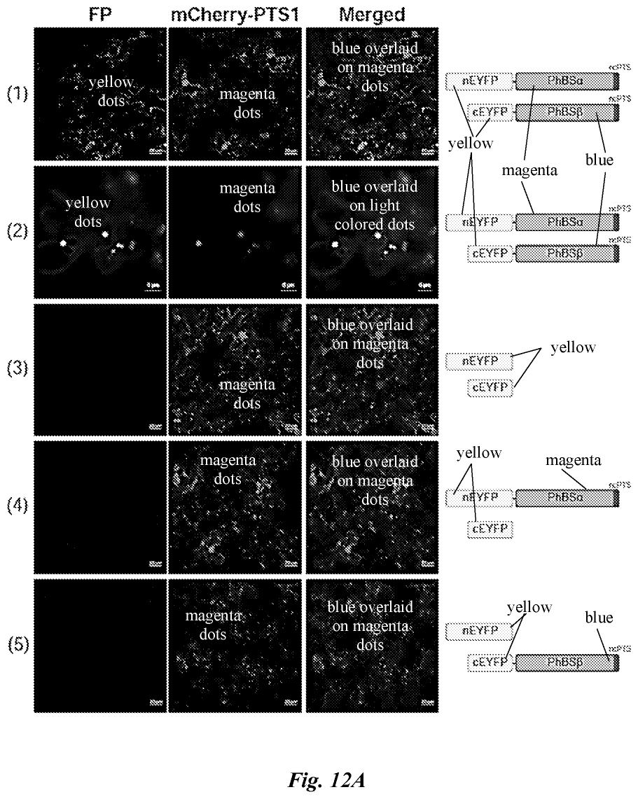

Aliquots of fractions from prokaryotic expression and purification of MBP-tagged PhBS submits were subjected to 15% SDS-PAGE analysis, including total soluble bacterial lysate after IPTG induction (Crude), fractions passed through amylose resin (Flow through), and ˜2 μg of purified protein after elution with 10 mM maltose solution (Purified). The experiment was repeated at least six times with similar results. Neither of the two purified recombinant proteins displayed benzaldehyde synthase activity when tested alone in assay mixture containing benzoyl-CoA and NADPH (FIG. 11A; triangle indicates the position of the MBP-tagged PhBS subunits); however, benzaldehyde was efficiently formed when the two subunit proteins were mixed in equal amounts (e.g., a 1:1 ratio) (FIG. 4A). MBP is about 42.5 kDa in size for reference.

Purified PhBS (1:1 ratio between α and β subunits) was also incubated with benzoyl-CoA and its structural analogs including cinnamoyl-CoA, para-coumaroyl-CoA, caffeoyl-CoA, feruloyl-CoA, and sinapoyl-CoA, and the formation of cinnamaldehyde and coniferaldehyde by purified PhCCR1 was used as a positive control. FIG. 4B shows the combined extracted ion current (EICs) chromatograms of mass units 106 (benzaldehyde), 128 (internal standard (IS)), 131 (cinnamaldehyde), and 178 (coniferaldehyde). The response of internal standard in each run was set as 100%. The purified recombinant petunia PhCCR1 that was used as a positive control successfully reduced synthesized hydroxycinnamoyl-CoA thioesters to their respective aldehydes; however, no corresponding aldehyde production was detected when these CoA esters were incubated with PhBSα and PhBSβ (1:1 ratio) and NADPH (FIG. 4B). These results support that both subunits are required for activity of PhBS, which displays strict substrate selectivity for benzoyl-CoA.

To analyze substrate specificity of PhBS, CoA esters were enzymatically synthesized from cinnamic acid, para-coumaric acid, caffeic acid, ferulic acid and sinapic acid using purified recombinant petunia 4-coumarate:CoA ligase (Ph4CL1) purified using the protocols described herein (FIG. 11B; triangle indicates the position of the Ph4CL1 protein). As shown in FIG. 11B, the purified Ph4CL1 was subjected SDS-PAGE analysis to confirm purity, with the total soluble bacterial lysate after IIPTG induction identified as “Crude” and ˜2 μg of purified protein after elution with maltose solution identified as “Purified”. The experiment was repeated at least six times with similar results.

Additional PhBS biochemical characterization further revealed that the apparent Kₘ for benzoyl-CoA was 677.2±80.5 μM and the catalytic efficiency (k_{cat}/Kₘ) was 13.9±1.8 M⁻¹ s⁻¹ (Table 4).

TABLE 4
Kinetic parameters of recombinant BS from four species.

| | Kₘ | Vₘₐₓ | k_{cat} | k_{cat}/Kₘ |
|---|---|---|---|---|
| Organism | (μM) | (nKat/mg) | (s⁻¹)^{a} | (mM⁻¹ s⁻¹) |
| Petunia hybrida^{b} | 677.2 ± 80.5 | 63.5 ± 2.7 | 9.39 ± 0.40 | 13.9 ± 1.8 |
| Petunia hybrida^{c} | 1342.0 ± 43.1 | 67.1 ± 0.9 | 9.92 ± 0.14 | 7.4 ± 0.3 |
| Arabidopsis tholiana^{b} | 242.8 ± 28.2 | 14.4 ± 0.8 | 2.12 ± 0.08 | 8.7 ± 1.1 |
| Prunus dulcis^{b} | 629.4 ± 67.7 | 35.4 ± 1.3 | 5.26 ± 0.20 | 8.4 ± 1.0 |
| Solanum lycopersicum^{b} | 767.3 ± 216.5 | 13.2 ± 3.0 | 1.96 ± 0.23 | 2.5 ± 0.8 |

Data are means ± error.
Error values are standard errors as derived from nonlinear fit analyses.
n = 3 technical replicates.
^{a}Assuming one catalytic center per heterodimer of BS.
^{b}Kₘ data were for benzoyl-CoA, measured at 4 mM NADPH.
^{c}Kₘ date were for NADPH, measured at 3.6 mM benzoyl-CoA.

These values lie within the range of catalytic efficiencies previously reported for other proteins (Ph4CL1, PhCNL, PAAS) responsible for the formation of volatile benzenoid/phenylpropanoid compounds in petunia flowers. Klempien et al., Contribution of CoA ligases to benzenoid biosynthesis in petunia flowers, The Plant Cell 24: 2015-2030 (2012); Kaminaga et al., Plant phenylacetaldehyde synthase is a bifunctional homotetrameric enzyme that catalyzes phenylalanine decarboxylation and oxidation, J Biological Chemistry 281: 23357-23366 (2006). The apparent Km value for NADPH was 1342.0±43.1 μM (Table 4).

### Example 4

### Benzaldehyde Synthase is a Peroxisomal Heterodimeric Enzyme

To examine if the two subunits interact with each other to form a heterodimeric benzaldehyde synthase, pull-down, yeast two-hybrid (Y2H) and bimolecular fluorescence complementation (BiFC) assays were used.

For pull-down assays, PhBS was purified from a mixture of bacterial lysates containing MBP-tagged α-subunit (MBP-PhBSα) and C-terminal His-tagged β-subunit (PhBSβ-His) using amylose resin. PhBSβ-His was co-purified with MBP-PhBSα as evidenced by two bands visible on the SDS-PAGE (FIG. 4C; the asterisk (*) indicating the position of MBP-PbBSα and the triangle indicates the position of PhBSβ-His). More specifically, purified MBP-PhBSα was digested with Factor Xa protease, incubated with purified PhBSβ-His, and the complex was again purified using a Ni-nitrilotriacetic acid (Ni-NTA) agarose column and analyzed by SDS-PAGE. Since tag-free PhBSα and PhBSβ-His have the same apparent sizes, purified PhBS showed only one band on the SDS-PAGE but possessed benzaldehyde synthase activity which indicates the presence of both subunits (FIG. 4D; the asterisk (*) indicating the position of MBP-PhBSα, the double-asterisk (**) indicating the position of free MBP tag, and the triangle representing the position of PhBSβ-His and untagged PhBSβ interactions).

The Y2H assays were performed to detect PhBSα and PhBSβ interactions. Coding sequences of both PhBSα and PhBSβ were PCR amplified using gene-specific primers (see Table 1 of FIG. 24) and cloned into EcoRI sites of the GAL4 Y2H system plasmids pGADT7-AD and pGBKT7-BD, containing the activation domain (AD) and the DNA-binding domain (BD), respectively, using ClonExpress II One Step Cloning Kit (Vazyme Biotech Co., Piscataway, NJ). Different combinations of AD and BD plasmids were co-transformed into yeast strain Y2H Gold and spotted at increasing dilutions on -leu/-trp double drop out (DDO) (i.e., non-selective) medium. After incubation at 30° C. for 3 days, individual large colonies (>2 mm diameter) that survived on DDO plates were resuspended in 100 μL sterile TE buffer (10 mM Tris, 1 mM EDTA, pH 7.5), subjected to a series of stepwise dilutions and blotted at increasing dilutions onto -leu/-trp/-his triple drop out (TDO) (i.e., selective) medium to test their growth response at 30° C. At least 10 colonies from each combination were tested on selective medium with the same results.

All TDO plates were image documented for colony growth 48 hours after blotting to control false positive growth, which is common after prolonged incubation. Constructs containing the PhBS subunits were also co-transformed with empty vectors to exclude unspecific interactions between PhBS subunits and AD/BD domains. pGADT7-T/pGBKT7-53 and pGADT7-T/pGBKT7-lam were used as positive and negative controls of the GAL4 Y2H system.

The Y2H assays showed that the two subunits directly interacted with each other when PhBSα was fused to GAL4 DNA-BD and PhBSβ was fused to AD. However, a negative growth response was observed when BD and AD were switched between the two subunits probably due to a spatial blockage of the protein-protein interaction interface (FIG. 4E).

To analyze whether the two subunits can form heterodimers in vivo, BiFC was performed, which also allowed for the detection of subcellular localization of interacting proteins. Citovsky et al., Localizing protein-protein interactions by bimolecular fluorescence complementation in planta, Methods 45: 196-206 (2008). Protein targeting prediction program WoLF PSORT predicted a possible peroxisomal localization for both subunits, despite neither containing the most common peroxisomal targeting sequence (PTS1). Lingner et al., Identification of novel plant peroxisomal targeting signals by a combination of machine learning methods and in vivo subcellular targeting analyses, The Plant Cell 23: 1556-1572 (2011).

To examine the subcellular localization of the PhBS subunits, full-length CDSs of each subunit was PCR amplified using gene-specific primers (see Table 1 of FIG. 24) and cloned into binary plasmids pK7WGF2 and pCNHP-EYFP, which expressed fusion proteins with N-terminal green fluorescent protein (GFP) and C-terminal enhanced yellow fluorescent protein (EYFP), respectively. For BiFC, PhBSα and PhBSβ6 CDSs were amplified and cloned into plasmids pCNHP-nEYFP-C and pCNHP-cYFP-C, which expressed fusion proteins with either N-terminal half of EYFP (nEYFP) or C-terminal half of EYFP (cEYFP) at their N-terminus, respectively. The resulting constructs were used for transient expression in N. benthamiana leaves.

Transformation of constructs into A. tumefaciens and infiltration of cell cultures in tobacco leaves were performed as described above except that the final OD₆₀₀ of the culture was adjusted to 0.6. Plasmid expressing mCherry-labelled peroxisomal marker obtained from ABRC (ABRC stock: CD3-984) was co-infiltrated with PhBS subunit expression constructs. 48 hours after infiltration, the fluorescent signals in the leaves were imaged using a Zeiss LSM-880 laser-scanning confocal microscope (Zeiss, Thornwood, NY, USA). The excitation wavelength and emission bandwidth recorded for each fluorescent protein as well as chlorophyll autofluorescence were optimized by the default presets in the ZEN 2.6 software (Carl Zeiss AG, Jena, Germany) and were as follows: EYFP (excitation 514 nm, emission 519˜583 nm), GFP (excitation 488 nm, emission 493˜556 nm), mCherry (excitation 561 nm, emission 580˜651 nm), chlorophyll autofluorescence (excitation 633 nm, emission 652˜721 nm).

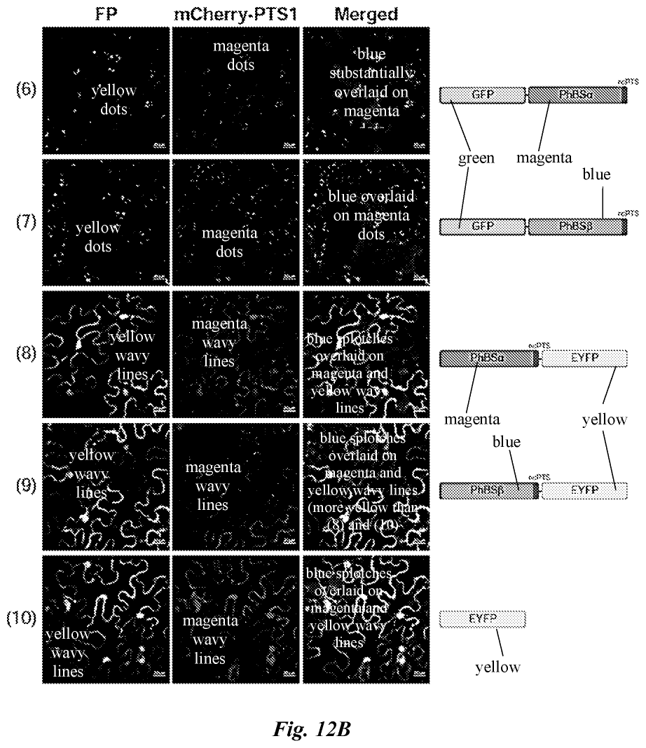

The co-expression of PhBSα tagged with the nEYFP (nEYFP-PhBSα) with PhBSβ tagged with cEYFP (cEYFP-PhBSβ) in Nicotiana benthamiana resulted in a reconstituted fluorescent signal, which was detected in peroxisomes, confirming that PhBS is a peroxisomal heterodimeric enzyme (FIG. 12A). To further verify the subcellular localization of PhBS subunits in planta, the coding region of each subunit was fused to either C-terminus of GFP or N-terminus of EYFP reporter gene and transiently co-expressed in N. benthamiana leaves with peroxisomal mCherry-labeled marker protein. The N-terminal fusion proteins, GFP-PhBSα and GFP-PhBSβ, showed peroxisomal localization while the C-terminal fusion proteins, PhBSα-EYFP and PhBSβ-EYFP, with blocked peroxisomal targeting signals failed to enter peroxisomes and accumulated in the cytosol instead (FIG. 12B).

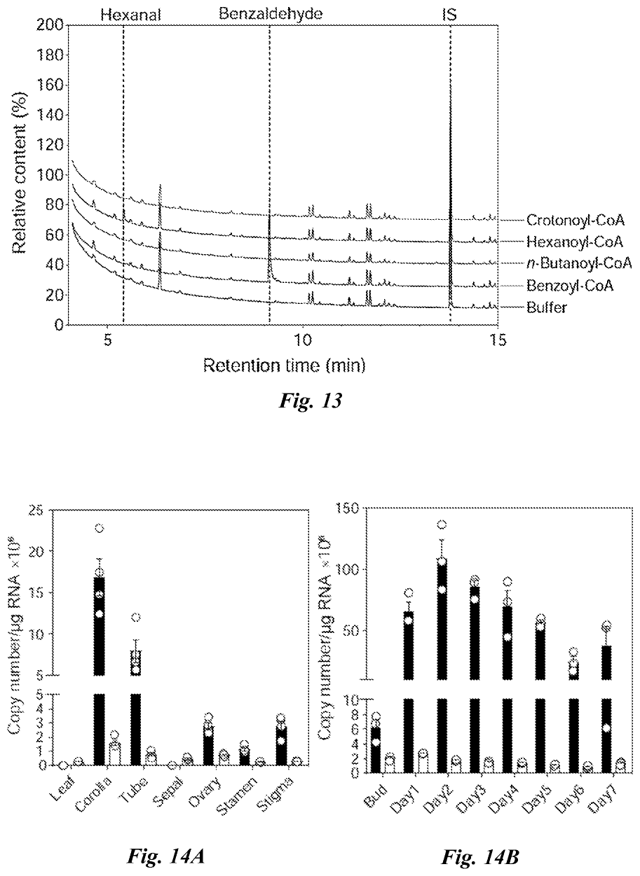

Given the peroxisomal localization of PhBS, it was also analyzed if purified PhBS could be involved in metabolism of short-chain fatty acyl-CoA esters. Purified PhBS (1:1 ratio between α and β subunits) was incubated with 200 μM benzoyl-CoA or 1 mM fatty acyl-CoA, including n-butanoyl-CoA, hexanoyl-CoA, and crotonoyl-CoA. All reactions were carried out at 28° C. for 1 hour. As shown in FIG. 13, only trace amounts of hexanal were detected with hexanoyl-CoA substrate (total ion currents (TICs) of scan mode (m z 35 to 250 shown).

### Example 5

### The Expression of PhBSα and PhBSβ6 is Differentially Regulated in Flowers

To assess the involvement of genes encoding PhBSα and PhBSβ in benzaldehyde formation, their spatial, developmental and temporal expressions were analyzed using quantitative real-time polymerase chain reaction (qRT-PCR) with gene-specific primers. Unexpectedly, expression of PhBSα was significantly higher than that of PhBSβ and only PhBSα displayed expression profiles typical for genes involved in scent production. Colquhoun et al., Petunia floral volatile benzenoid/phenylpropanoid genes are regulated in a similar manner, Phytochemistry 71: 158-167 (2010).

Sample collection, RNA isolation and quantitative real-time PCR (qRT-PCR) were performed as described in Klempien (2012), supra. Briefly, samples were collected from tissues and time points indicated in the text and RNA was extracted using the Spectrum Plant Total RNA Kit (Millipore Sigma, St. Louis, MO). About 1 μg of total RNA was reverse transcribed to first strand cDNA in 10 μl reaction using the EasyScript cDNA synthesis kit (Applied Biological Materials Inc., Vancouver, Canada). Individual qRT-PCR reactions were performed in 5 μL of Fast SYBR Green Master Mix (Applied Biosystems, Waltham, MA) with the gene-specific primers shown in Table 1 of FIG. 24 using a StepOne Real-Time PCR System (Applied Biosystems, Waltham, MA). PhBSα and PhBSβ were expressed as a copy number of transcripts per microgram of total RNA×10⁶.

For relative expression quantification, Elongation factor 1-alpha (PhEF1α) and Actin 2 (AtACT2, AT3G18780) were used as internal reference genes for petunia and Arabidopsis cDNAs, respectively. Absolute quantities of transcripts were calculated based on standard curves generated from purified templates of the corresponding CDSs and expressed as copy numbers per microgram of total RNA.

FIG. 5A shows the changes in PhBSα (white box) and PhBSβ (black box) transcript levels during a normal light/dark cycle in petunia corolla harvested 1-day post-anthesis (15:00) to day 3 post-anthesis (3:00). White and gray areas of the graph in FIG. 5A correspond to light and dark cycles, respectively.

Tissue-specific (FIG. 14A) and developmental (FIG. 14B) expression of PhBSα and PhBSβ was also assessed. As shown in FIG. 14A, PhBSα (black bar) was highly expressed in petal limbs and tubes, the parts of the flower that were previously shown to be primarily responsible for scent emission in petunia, with low transcript levels in leaves and sepals. Underwood et al., Ethylene-regulated floral volatile synthesis in petunia corollas, Plant Physiology 138: 255-266 (2005). Additionally, as shown in FIG. 14B, PhBSα mRNA levels in corolla limbs were developmentally regulated, increasing from bud to day 2 post-anthesis and changing rhythmically during a daily light/dark cycle, with a peak before 19:00, which preceded the peak of benzaldehyde emission. Boatright et al. (2014), supra; Liao et al. (2021), supra.

In contrast, PhBSβ was constitutively expressed in all tissues examined. The transcript levels of PhBSβ in limbs moderately fluctuated during flower development (FIG. 14B) and a daily light/dark cycle (FIG. 5A) and did not correlate with the known pattern of benzaldehyde emission. This data supports that PhBSα determines the transcriptional specificity of PhBS. Boatright et al. (2014), supra.

### Example 6

### PhBS is Responsible for Benzaldehyde Synthesis in Planta

To investigate the in vivo function of PhBS, the expression of both PhBSα and PhBSβ were downregulated in flowers by virus induced gene silencing (VIGS), a technique that has been previously successful in studying floral scent in petunia. Spitzer et al. (2007), supra; Spitzer-Rimon et al., EOBII, a gene encoding a flower-specific regulator of phenylpropanoid volatiles' biosynthesis in Petunia, The Plant Cell 22: 1961-1976 (2010); Cna'ani et al., Two showy traits, scent emission and pigmentation, are finely coregulated by the MYB transcription factor PH4 in petunia flowers, New Phytologist 208: 708-714 (2015); Liu et al., PhERF6, interacting with EOBI, negatively regulates fragrance biosynthesis in petunia flowers, New Phytologist 215: 1490-1502 (2017). Benzaldehyde emission was then assessed.

A 301-bp fragment of each gene was placed in a tandem in the Tobacco rattle virus (TRV)-based vector downstream of a 300 bp fragment of Phytoene Desaturase (PDS), the silencing of which was used as a visual marker for VIGS effectiveness, using protocols commonly known in the art. PDS silencing alone did not affect the benzaldehyde levels, which were similar to that previously detected in wild-type flowers. Body et al., Caterpillar chewing vibrations cause changes in plant hormones and volatile emissions in Arabidopsis thaliana, Frontiers in Plant Science 10: 810 (2019). FIG. 5B shows the transcript levels of PhBSα and PhBSβ in pds control (black bars) and pds-bsα-bsβ (white bars) in 2-day-old VIGS flowers determined by qRT-PCR at 21:00 hours, presented relative to the corresponding levels in pds control set as 1. On average, a 61% and 66% reduction were observed in PhBSα and PhBSβ mRNA levels, respectively, in flowers collected from mosaic photobleached branches of pds-bsα-bsβ VIGS plants relative to that in pds control plants.

BS activities in crude extracts prepared from corollas of 2-day-old VIGS flowers harvested at 21:00 hours were also assessed using the previously described protocols, as was benzaldehyde emission (volatiles collected from 2-day-old VIGS flowers from 20:00 hours to 21:00 hours). Consistent with the decrease in PhBS expression see in FIG. 5B, benzaldehyde synthase activity in petal crude extracts and benzaldehyde emission was reduced by 71.2% and 68.3%, respectively, in pds-bsα-bsβ flowers relative to pds control (FIGS. 5C and 5D), which provides strong genetic evidence that the heterodimeric PhBS is responsible for benzaldehyde formation in petunia.

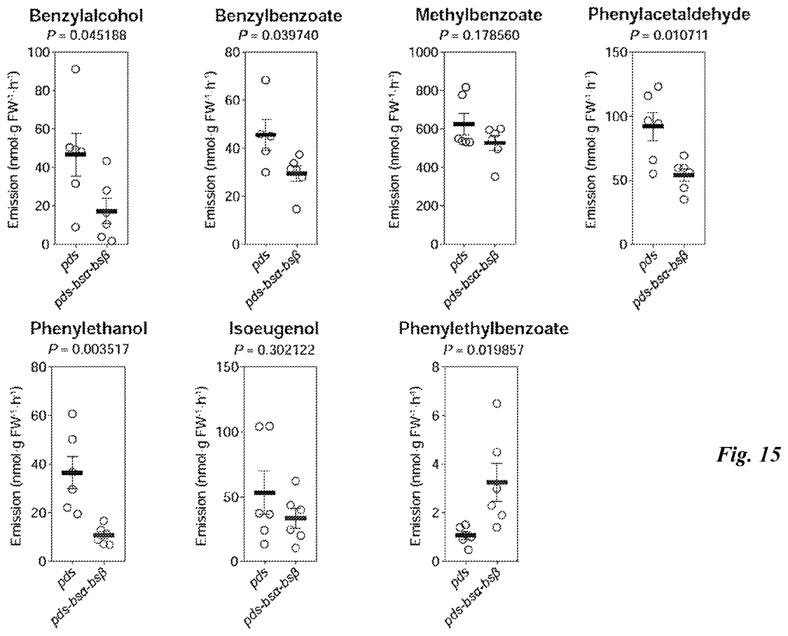

In addition, the emission rates of several individual VOCs were measured to determine the effect, if any, of PhBS downregulation thereon. As shown in FIG. 15, benzylalcohol (the product of benzaldehyde reduction) and benzylbenzoate (which relies on benzylalcohol as a co-substrate) were reduced on average by 62.9% and 35.6%, respectively, in pds-bsα-bsβ flowers relative to control. In contrast, emission of methylbenzoate, the biosynthesis of which depends on benzoic acid formed by both β-oxidative and non-β-oxidative pathways, remained unaffected. Interestingly, a 41.2% and 70.7% decrease on average was observed in phenylacetaldehyde and phenylethanol emission, respectively, while phenylethylbenzoate emission was increased by 3.07-folds (FIG. 15). These results suggest that the reduced benzaldehyde production in the pds-bsα-bsβ flowers (e.g., due to the downregulation of PhBS) led to accumulation of benzyl-CoA and redirection of flux towards production of phenylethylbenzoate, which increased the consumption of phenylethanol (and its substrate, phenylacetaldehyde), thereby decreased the overall emission of such VOCs. No statistically significant changes were observed in isoeugenol emission (FIG. 15).

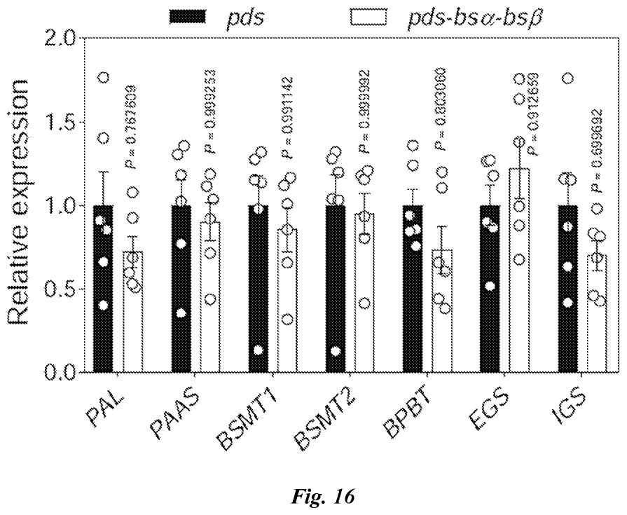

Analysis of expression of scent biosynthetic genes by qRT-PCR revealed that their mRNA levels were unchanged in VIGS flowers relative to control (FIG. 16).

To further evaluate the PhBS biosynthetic capacity in planta, both subunits α and β were expressed in N. benthamiana leaves, a tissue that does not naturally produce detectable amount of benzaldehyde. In addition, the complete benzaldehyde β-oxidative biosynthetic pathway was reconstituted in N. benthamiana by expressing together with the PhBS four additional genes encoding phenylalanine ammonia lyase 1 (PhPAL1), PhCNL, PhCHD and PhKAT. Klempien et al. (2012), supra; Qualley et al., Completion of the core 0-oxidative pathway of benzoic acid biosynthesis in plants. Proceedings of the National Acad of Sciences of the U.S.A. 109: 16383-16388 (2012); Moerkercke et al., A plant thiolase involved in benzoic acid biosynthesis and volatile benzenoid production, The Plant J. 60: 292-302 (2009). FIG. 5E shows a biosynthetic pathway for benzaldehyde in petunia flowers and the enzymes used for pathway reconstitution.

The coding sequences of PhPAL1, PhCNL, PhCHD, PhKAT, PhBSα (SEQ ID NO: 1) and PhBSβ (SEQ ID NO: 2) were PCR amplified with gene-specific primers (see Table 1 of FIG. 24) from petunia flower cDNAs and cloned into binary vector pCNHP using ClonExpress II One Step Cloning Kit (Vazyme Biotech Co., Piscataway, NJ). After sequence verification, plasmids were transformed into Agrobacterium tumefaciens strain GV3101. Single colonies for each construct were picked and cultured at 28° C. in 3 mL of LB medium supplied with 50 mg/L rifampicin, 50 mg/L gentamycin and 50 mg/L kanamycin to an OD₆₀₀ of about 2.0. Bacterial cultures were pelleted, washed with a 10 mM MgCl₂ solution containing 200 μM acetosyringone, and incubated in the same solution for additional 2 hours at room temperature. Before infiltration, cultures were mixed to reach a final OD₆₀₀ of 1.0 for each of the constructs used.

The suspension was injected into the abaxial surface of 4 to 6-week-old N. benthamiana leaves with a needleless syringe. Infiltrated plants were grown under dim light for 3 days before leaves were detached and submerged in 150 mM Phe solution for 24 hours. Leaf tissues were then ground into fine powder in liquid nitrogen. 200 mg of ground tissue were extracted twice with 500 μL of DCM containing 1 μg naphthalene (internal standard), concentrated to about 200 μL and subjected for GC-MS analysis.

To release benzylalcohol from its potential glucosides, 200 mg of ground tissue was suspended in 500 μL phosphate-citrate buffer (150 mM, pH 5.0) and 100 μL Viscozyme® L (Sigma-Aldrich, St. Louis, MO) was added in accordance with the protocols described in Boachon et al., Natural fumigation as a mechanism for volatile transport between flower organs, Nature Chemical Biology 15: 583-588 (2019). Samples were incubated at 37° C. for 6 hours with frequent mixing. Metabolites were extracted twice with DCM and subjected for GC-MS analysis as described above.

Results are shown in FIGS. 5F-5I, with FIG. 5F showing a GC-MS analysis of infiltrated N. benthamiana leaves after mock feeding, FIG. 5G showing a GC-MS analysis of infiltrated tobacco leaves after Phe feeding for 24 hours, and FIG. 5H showing a GC-MS analysis of infiltrated tobacco leaves after Phe feeding and Viscozyme® treatment. Only trace amounts of benzaldehyde were detected in all three different combinations of enzymes 3 days after N. benthamiana leaves infiltration, which could be the result of substrate limitation (FIG. 5F). Indeed, feeding of transformed leaves with 150 mM Phe for 24 hours increased not only the levels of benzaldehyde, but also benzylalcohol in all groups, with the highest levels in leaves that express the entire pathway (FIG. 5G) suggesting that part of produced benzaldehyde was efficiently reduced to benzylalcohol.

Notably, leaves expressing the empty vector also showed an increase in benzaldehyde content, indicating the existence of some endogenous benzaldehyde biosynthetic capacity in tobacco. Indeed, a weak BS activity (<0.3 pKat·mg protein⁻¹) was detected in crude extracts of N. benthamiana leaves. Despite the low benzaldehyde levels detected, tobacco leaves with the introduced whole pathway produced 321.5 nmol·g FW⁻¹ more benzylalcohol than the leaves expressing the whole pathway without PhBS (1295±19.0 nmol·g FW⁻¹ versus 973.5±23.4 nmol·g FW⁻¹ in leaves infiltrated with the whole pathway and PAL-CNL-CHD-KAT, respectively) (FIGS. 5H and 5I). This result was consistent with the amount of benzylalcohol produced in leaves expressing only PhBS (327.9±14.3 nmol·g FW⁻¹) versus the empty vector control (26.2±0.7 nmol·g FW⁻¹) (FIG. 5H). More than 95% of detected benzylalcohol were found in its glycosylated form (FIGS. 5H and 5I), suggesting the presence of strong glycosylation activity in tobacco leaves.

### Example 7

### BS Homologs From Other Species Harbor BS Activity

To expand application of the inventive findings hereof about benzaldehyde biosynthesis to other plant species, phylogenetic analysis was performed using protein sequences of the less diverse BSβ subunits. PhBSβ homologs were found in many land plants including monocotyledonous, dicotyledonous species and Physcomitrella patens, most of which have only a single copy of BSβ6 in their genomes.

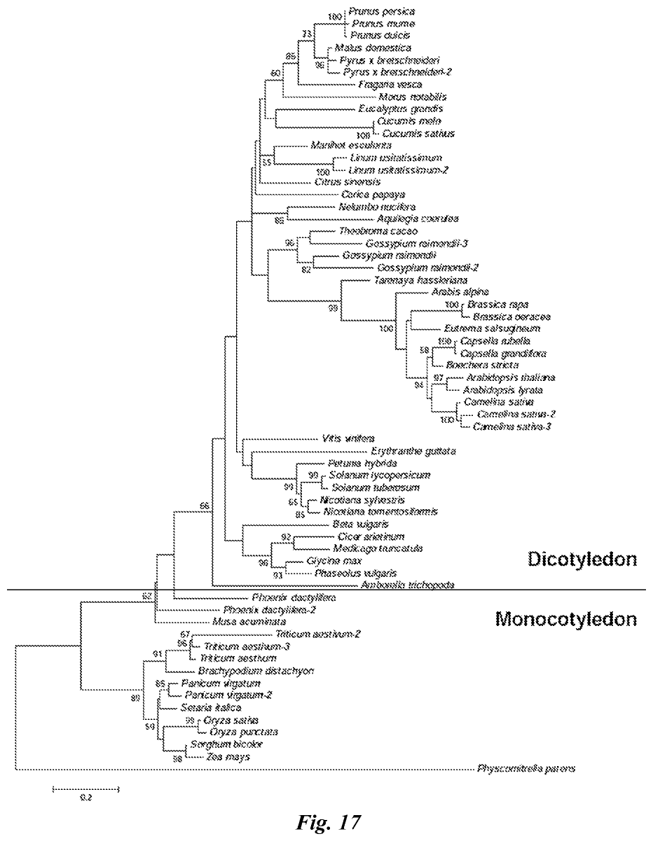

FIG. 17 shows a phylogenetic analysis of such PhBSβ homologs in land plants, obtained by the publicly available EggNOG Sequence search using the PhBSβ amino acid sequence (SEQ ID NO: 2). Huerta-Cepas et al., EggNOG 5.0: A hierarchical, functionally and phylogenetically annotated orthology resource based on 5090 organisms and 2502 viruses, Nucleic Acids Research 47: D309-D314 (2019).

Homologs of both PhBSα and PhBSβ from Petunia genus were also obtained by BLASTP search against published genomes on the publicly available Solanaceae Genome Project website. The amino acid sequences were aligned using MUSCLE algorithm in MEGA7 package. Kumar et al., MEGA7: Molecular evolutionary genetics analysis version 7.0 for bigger datasets, Molecular Biology & Evolution 33: 1870-1874 (2016).

As previously described, the Neighbor-Joining algorithm in MEGA7 program was used to build the phylogenetic tree shown on FIG. 10. The phylogenetic trees were tested with Bootstrap method for 1000 replications.

The tree with the highest log likelihood (−7504.55) is shown. The percentage of trees in which the associated taxa clustered together is shown next to the branches. Initial tree(s) for the heuristic search were obtained automatically by applying Neighbor-Join and BioNJ algorithms to a matrix of pairwise distances estimated using the JTT model, and then selecting the topology with superior log likelihood value. A discrete Gamma distribution was used to model evolutionary rate differences among sites (8 categories (+G, parameter=1.9404)).

Three representative organisms including tomato (Solanum lycopersicum), almond (Prunus dulcis), and Arabidopsis thaliana were chosen from the analysis to test the hypothesis that a heterodimeric enzyme consisting of a and β subunits is responsible for benzaldehyde formation in species other than Petunia. In tomato, benzaldehyde contributes to the fruit aroma, in almonds benzaldehyde accumulates to high levels in leaves, flowers, and young fruits, while low benzaldehyde production was found in Arabidopsis leaves. Baldwin et al., Effect of volatiles and their concentration on perception of tomato descriptors, J. Food Science 69: S310-S318 (2004); Nawade et al., Profiling of volatile terpenes from almond (Prunus dulcis) young fruits and characterization of seven terpene synthase genes, The Plant Science 287: 110187 (2019); Body et al. (2019), supra.

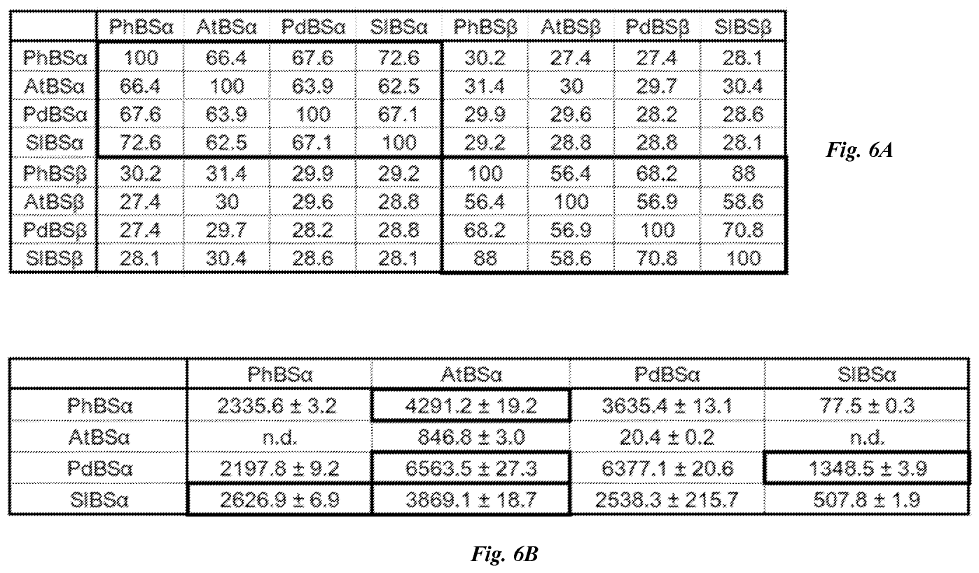

A protein sequence identity matrix between BS subunits from petunia (PhBS), Arabidopsis (AtBS), almond (PdBS) and tomato (SlBS) is shown in FIGS. 6A and 6B. Selected a subunits share 62.5% to 72.6% of amino acid identity, while β subunits are 56.4% to 88% identical with the Arabidopsis β subunit being the most distantly related (FIGS. 6A, 18A, and 18B).

To verify these homologs express characteristics and activities that equate with those of PhBS, the BSs of each species were purified using the protocols described herein and SDS-PAGE analysis of about 2 μg of purified MBP-tagged BS subunits from petunia, Arabidopsis, almond and tomato was performed to determine molecular weight. The experiment was repeated at least six times with similar results. All enzymes were successfully purified and FIG. 19A supports each purified subunit had equivalent molecular weights.

GC-MS analysis was also performed of the purified recombinant BS proteins formed in vitro by Arabidopsis (FIG. 19B), almond (FIG. 19C), and tomato (FIG. 19D). Like PhBS, benzaldehyde synthases from all three organisms (i.e., Solanum lycopersicum, Prunus dulcis, and Arabidopsis thaliana) showed activity only when two purified recombinant subunits were combined (FIGS. 19B-19D), indicating that the mechanism observed in Petunia was preserved in the identified homologs for benzaldehyde synthesis during the evolutionary process. Kinetic evaluation of these enzymes revealed that Arabidopsis AtBS and tomato SlBS had very similar apparent Km values for benzoyl-CoA, which were slightly lower than that of almond enzyme, PdBS, and PhBS (see Table 4). PhBS had the highest catalytic efficiency followed by PdBS, while AtBS and SlBS had nearly equal catalytic efficiencies (Table 4).

To determine whether benzaldehyde synthase a subunits could interact productively with β subunits from phylogenetically distant species, enzyme assays were performed with purified recombinant petunia, Arabidopsis, almond and tomato a and β subunits in different combinations (FIG. 6B). Out of the four homolog β subunits examined, only the AtBSβ subunit was not able to produce active enzymes upon interaction with PhBSα and SlBSα. Instead, the AtBSβ subunit formed a low activity hybrid heterodimer with PdBSα.

Since the inertness of PhBSα-AtBSβ hybrid could be the result of the inability of AtBSβ to form heterodimers, purified PhBSα, PhBSβ, AtBSβ and PhBSα-AtBSβ were subjected to size exclusion chromatography. 500 μL purified protein (˜ 500 μg) were loaded onto a Superdex 200 Increase 10/300 GL size exclusion column and eluted with PBS. The column was calibrated with the following markers: bovine thyroglobulin (670 kDa), bovine gamma globulin (158 kDa), chicken ovalbumin (44 kDa), horse myoglobulin (17 kDa), and vitamin B-12 (1.4 kDa).

At the concentration tested (˜1 mg/mL), both PhBSβ and AtBSβ exist predominantly as tetramers by themselves, while the PhBSα forms mostly multimers (FIG. 19E). Mixing of AtBSβ with PhBSα, prevented formation of PhBSα multimers, indirectly indicating that AtBSβ indeed physically interacts with PhBSα consistent with the other homologs (FIG. 19E). The other three R subunits formed active hybrid proteins with all a subunits, but the effect on activities of hybrid enzymes relative to the native benzaldehyde synthases was different. While interactions of PdBSβ and SlBSβ with PhBSα resulted in hybrid enzymes with activities almost identical to that of PhBS, interactions of the three tested β subunits with AtBSα promoted activities of hybrid proteins, increasing activity from 4.6- to 7.8-fold in AtBSα-SlBSβ and AtBSα-PdBSβ, respectively, relative to AtBS (FIG. 6B). In contrast, the activities of hybrid enzymes comprising PdBSα were up to 2.5-fold lower than that of PdBS enzyme, while activities of SlBSα-PhBSβ and SlBSα-PdBSβ were 6.5-fold lower and 2.7-fold higher, respectively, as compared with SlBS. Interestingly, the AtBSα-PdBSβ hybrid was as active as PdBS, but the opposite combination PdBSα-AtBSβ exhibited very low activity (FIG. 6B). Overall, the results support that BSα subunits can form an active enzyme with β subunits from phylogenetically distant species; however, not all BSβ subunits (e.g., Arabidopsis) can form active enzymes with a subunits. Moreover, the effect of BSβ subunits on the activities of hybrid enzymes depends on the origin of interacting a subunit.

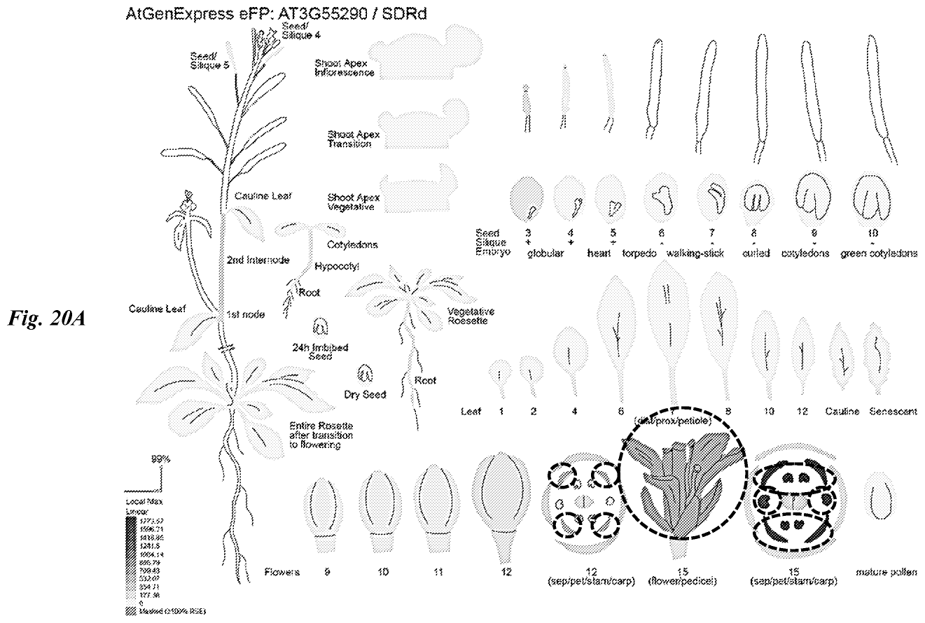

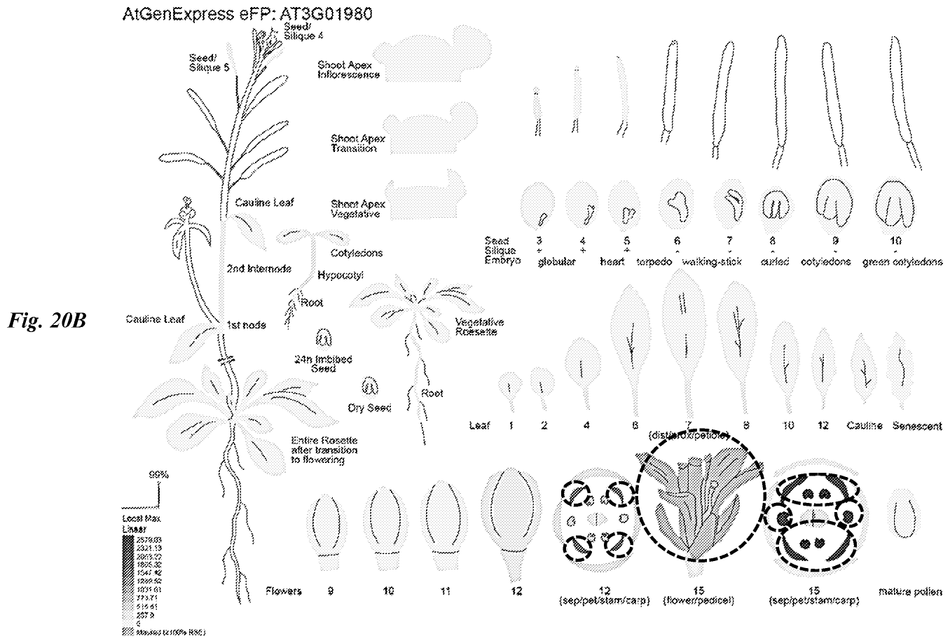

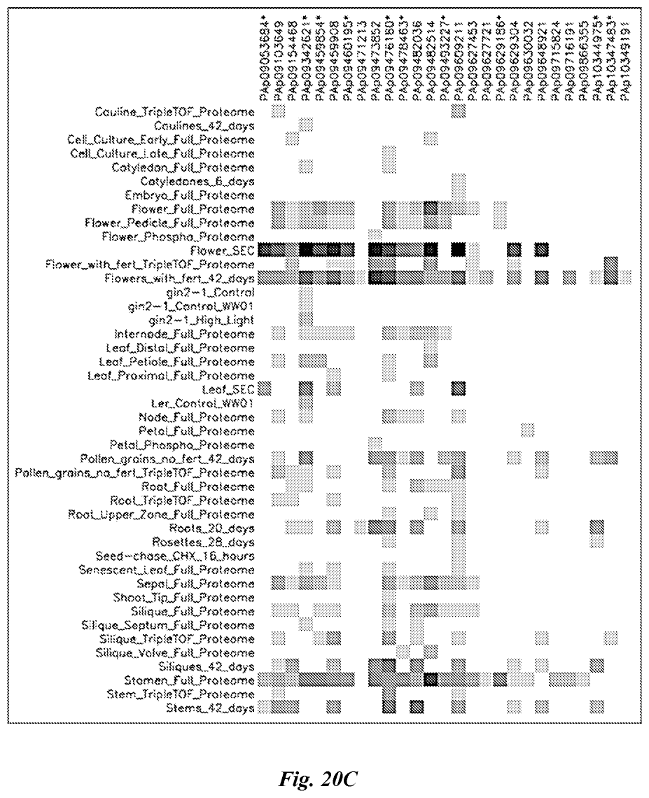

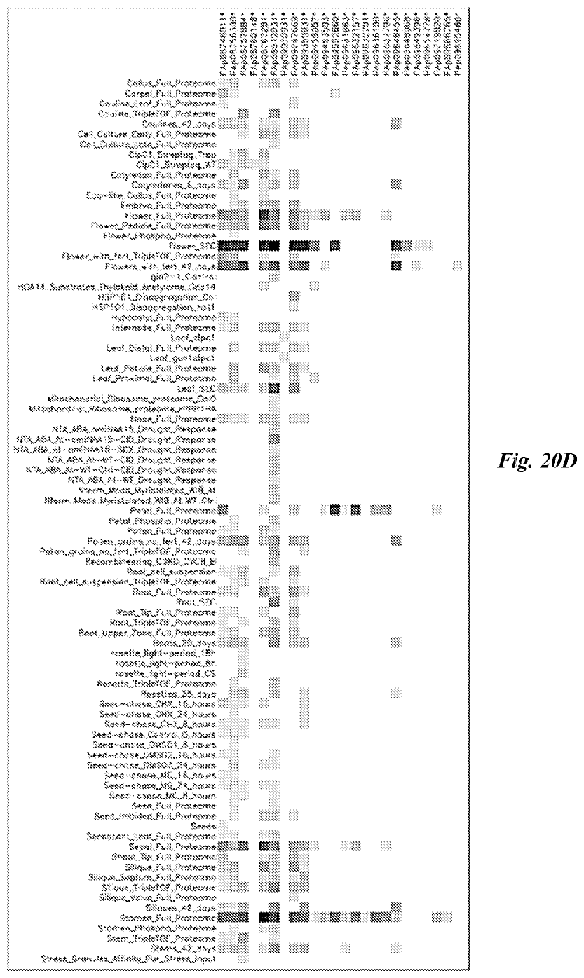

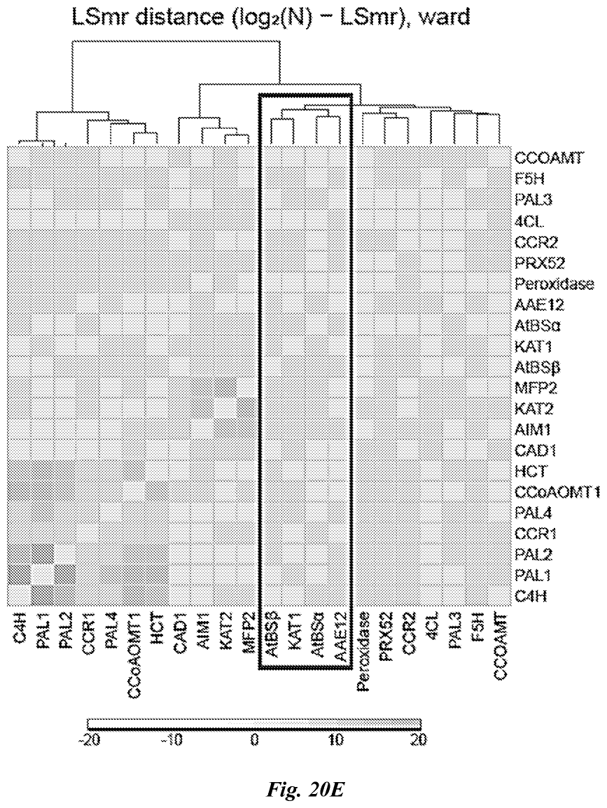

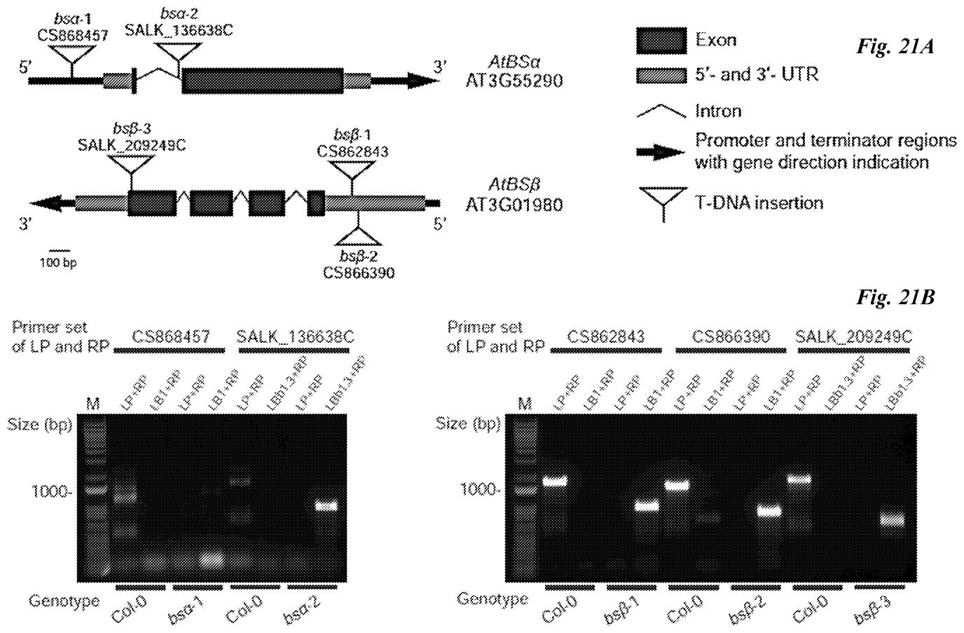

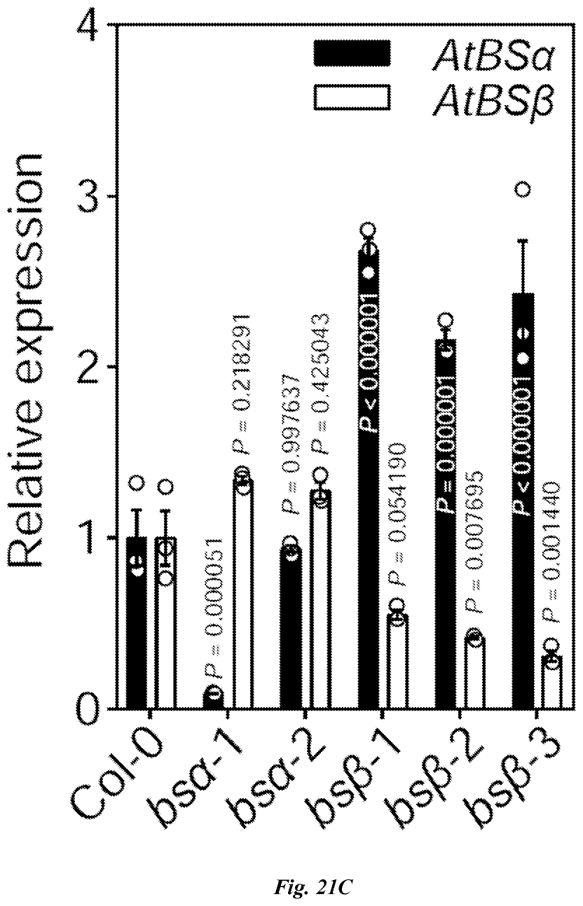

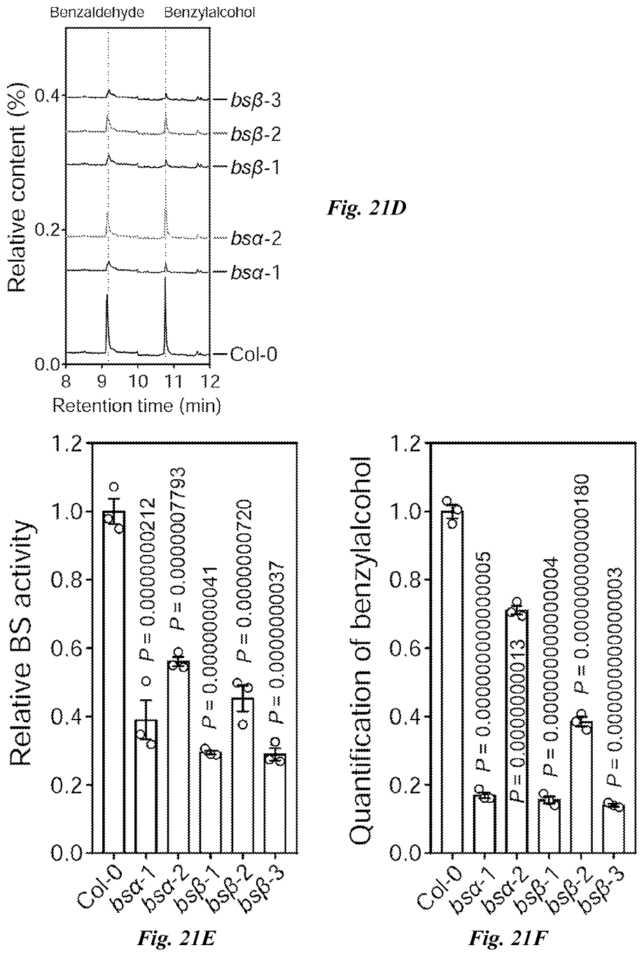

As the Arabidopsis genome contains AtBS genes, it was then analyzed if encoding a heterodimeric enzyme is responsible for benzaldehyde synthesis. A search of the Arabidopsis publicly available expression datasets and proteome database (Arabidopsis PeptideAtlas) revealed that AtBSα and AtBSβ6 are highly expressed in flowers at both transcriptional and translational levels with relatively low expression in leaves (FIGS. 20A-20E). In addition, the expression patterns of AtBSα and AtBSβ were more closely clustered with KAT1 and AAE12 (which encode a PhCNL homolog and are the core genes in the β-oxidation pathway), rather than with phenylpropanoid and lignin biosynthetic genes (FIG. 20E), which supports these two genes are likely associated with the β-oxidative benzoic acid biosynthetic pathway. Metabolic profiling of Arabidopsis flowers and analysis of BS activity revealed that benzaldehyde and benzylalcohol internal pools were below detection levels while weak BS activity (0.075 pKat·mg protein⁻¹ on average) as well as benzaldehyde reduction activity were present in crude flower extracts. Two T-DNA insertion lines of AtBSα (At3g55290; SEQ ID NO: 92) and three T-DNA insertion lines of AtBSβ (At3g01980; SEQ ID NO: 93) (FIG. 21A) were obtained from ABRC and their homozygosity was confirmed by genomic PCR analysis with gene-specific primers flanking the insertion sites, which failed to amplify the respective gene regions (FIG. 21B). More specifically, the two T-DNA insertion lines of AtBSα included GenBank accession number CS868457 (SEQ ID NO: 94), deposited with NCBI GenBank (accepted Dec. 15, 2007) and Nottingham Arabidopsis Stock Centre identification number SALK_136638C, and the three T-DNA insertion lines of AtBSβ6 included Nottingham Arabidopsis Stock Centre identification number SALK_209249C, GenBank accession number CS862843 (SEQ ID NO: 95) deposited with NCBI GenBank (accepted Dec. 15, 2007), and GenBank accession number CS866390 (SEQ ID NO: 96) deposited with NCBI GenBank (accepted Dec. 15, 2007).

All T-DNA insertions resulted in knockdown of respective genes with remaining transcript levels ranging from 9.2% to 55% relative to the wild-type plants, except for bsα-2 mutant, which showed no changes in AtBSα expression (FIG. 21C). Nevertheless, similar to other bs knockdowns, the bsα-2 mutant had significant reduction in BS activity (FIGS. 21D and 21E) likely due to post-transcriptional effect of intron with T-DNA insertion on protein expression despite the proper splicing of the intron. Rose & Last, Introns act post-transcriptionally to increase expression of the Arabidopsis thaliana tryptophan pathway gene PAT1, The Plant J. 11: 455-464 (1997). Interestingly, downregulation of AtBSβ resulted in upregulation of AtBSα, but not vice versa (FIG. 21C). As flower tissue contains benzaldehyde reduction activity, the production of benzaldehyde by flower crude extracts was rapidly converted to benzylalcohol. As such, the levels of benzyalcohol were lower in all bs mutants relative to wild-type plants and positively correlated with benzaldehyde levels in mutants (FIGS. 21D-21F). Taken together, these results support that heterodimeric BS is responsible for benzaldehyde biosynthesis in Arabidopsis (FIGS. 21D and 21E).

To test whether AtBS is involved in the biosynthesis of other aromatic aldehydes, hydroxycinnamoyl-CoA thioesters were incubated with purified AtBS enzyme, and the corresponding aldehyde formation was analyzed. Briefly, GC-MS analysis was performed of the products formed by AtBS from different hydroxycinnamoyl-CoA substrates. Purified AtBS (1:1 ratio between α and β subunits) was incubated with benzoyl-CoA and its structural analogs including cinnamoyl-CoA, para-coumaroyl-CoA, caffeoyl-CoA, feruloyl-CoA, and sinapoyl-CoA. All CoA esters except benzoyl-CoA were synthesized using Ph4CL1 and their corresponding free acids. Formation of cinnamaldehyde and coniferaldehyde by purified PhCCR1 was used as a positive control. Shown in FIG. 22 are the combined EICs of mass units 106 (benzaldehyde), 128 (internal standard), 131 (cinnamaldehyde), and 178 (coniferaldehyde). Similar to petunia enzyme, AtBS exhibited strict substrate specificity towards benzoyl-CoA.

1. A method for producing benzaldehyde comprising:
providing a biosynthesis platform comprising a first nucleic acid sequence encoding a benzaldehyde synthase alpha (BSα) subunit and a second nucleic acid sequence encoding a benzaldehyde synthase beta (BSβ) subunit such that the first and second nucleic acid sequences are overexpressed in the biosynthesis platform; and
subjecting the biosynthesis platform to conditions such that benzaldehyde is produced.

2. The method of claim 1, further comprising isolating the benzaldehyde from the biosynthesis platform.

3. The method of claim 1, further comprising:
transforming eukaryotic cells or microbes with a vector carrying the first nucleic acid sequence under conditions that allow for the overexpression of the BSα subunit;
transforming eukaryotic cells or microbes with a vector carrying the second nucleic acid sequence under conditions that allow for the overexpression of the BSβ subunit;
selecting transformants that overexpress both BSα and BSβ subunits; and
growing the transformants to facilitate de novo production of benzaldehyde in the biosynthesis platform.

4. The method of claim 1, further comprising:
transforming a first population of microbes with a vector carrying the first nucleic acid sequence under conditions that allow for the overexpression of the BSα subunit;
transforming a second population of microbes with a vector carrying the second nucleic acid sequence under conditions that allow for the overexpression of the BSβ subunit;
selecting transformants that overexpress the BSα subunit from the first population of microbes;
selecting transformants that overexpress the BSβ subunits from the second population of microbes; and
mixing the BSα subunits from the first population of microbes with the BSβ subunits from the second population of microbes to produce benzaldehyde.

5. The method of claim 1, wherein the first nucleic acid sequence, the second nucleic acid sequence, or both the first and second nucleic acid sequences is/are heterologous to the platform.

6. The method of claim 1, wherein:
(i) the first nucleic acid sequence is or comprises a nucleotide sequence of SEQ ID NO: 3, SEQ ID NO: 101, SEQ ID NO: 103, SEQ ID NO: 105, or a nucleotide sequence having at least 50% identity to SEQ ID NO: 3, SEQ ID NO: 101, SEQ ID NO: 103, or SEQ ID NO: 105;
(ii) the second nucleic acid sequence is or comprises a nucleotide sequence of SEQ ID NO: 4, SEQ ID NO: 102, SEQ ID NO: 104, SEQ ID NO: 106, or a nucleotide sequence having at least 50% identity to SEQ ID NO: 4, SEQ ID NO: 102, SEQ ID NO: 104, or SEQ ID NO: 106;
(iii) the first nucleic acid sequence encodes SEQ ID NO: 1, SEQ ID NO: 5, SEQ ID NO: 6, SEQ ID NO: 7 or a functional fragment or homolog of SEQ ID NO: 1, SEQ ID NO: 5, SEQ ID NO: 6, or SEQ ID NO: 7; or
(iv) the second nucleic acid sequence encodes SEQ ID NO: 2, SEQ ID NO: 8, SEQ ID NO: 9, SEQ ID NO: 10, or a functional fragment or homolog of SEQ ID NO: 2, SEQ ID NO: 8, SEQ ID NO: 9, or SEQ ID NO: 10.

7. The method of claim 1, wherein:
the first nucleic acid sequence encodes SEQ ID NO: 1, SEQ ID NO: 5, SEQ ID NO: 6, SEQ ID NO: 7 or a functional fragment or homolog of SEQ ID NO: 1, SEQ ID NO: 5, SEQ ID NO: 6, or SEQ ID NO: 7; and
the second nucleic acid sequence encodes SEQ ID NO: 2, SEQ ID NO: 8, SEQ ID NO: 9, SEQ ID NO: 10, or a functional fragment or homolog of SEQ ID NO: 2, SEQ ID NO: 8, SEQ ID NO: 9, or SEQ ID NO: 10.

8. The method of claim 1, wherein the biosynthesis platform comprises genetically engineered microbes or genetically engineered eukaryotic cells, tissues, organs, or organisms.

9. The method of claim 8, wherein the biosynthesis platform comprises a transgenic plant, a transgenic plant cell, a transgenic plant tissue, and/or a transgenic plant organ.

10. The method of claim 2, wherein the biosynthesis platform comprises a transgenic plant, a transgenic plant cell, a transgenic plant tissue and/or a transgenic plant organ, and isolating the benzaldehyde from the biosynthesis platform comprises isolating the benzaldehyde from the transgenic plant, the transgenic plant cell, the transgenic plant tissue, and/or the transgenic plant organ after growth.

11. The method of claim 8, wherein the biosynthesis platform comprises genetically engineered algae, insect cells, or animal cells.

12. The method of claim 10, wherein the transgenic plant is, or the transgenic plant cell, the transgenic plant tissue, and/or the transgenic plant organ is obtained from, Petunia hybrida, Nicotiana benthamiana, or Prunus dulcis.

13. The method of claim 1, wherein the biosynthesis platform comprises genetically engineered microbes of an Escherichia coli strain, a Saccharomyces cerevisiae strain, or a Pichia pastoris strain in a fermentation medium.

14. The method of claim 2, wherein the biosynthesis platform comprises genetically engineered microbes in a fermentation medium and isolating the benzaldehyde comprises recovering the benzaldehyde from the fermentation medium after fermentation.

15. The method of claim 1, wherein the first nucleic acid sequence is from a first species and the second nucleic acid sequence is from a second species.

16. The method of claim 1, wherein the BSα and BSβ subunits together form a heterodimeric enzyme.

17. The method of claim 1, further comprising supplying benzoyl-CoA, nicotinamide adenine dinucleotide phosphate (NADPH), or both benzoyl-CoA and NADPH to the biosynthesis platform.

18. The method of claim 4, further comprising supplying benzoyl-CoA, NADPH, or both benzoyl-CoA and NADPH to the mixture of the BSα and BSβ subunits.

19. The method of claim 18, further comprising purifying the BSα and Bsβ subunits.

20. The method of claim 1, wherein the molar ratio of the BSα subunits to BSβ subunits is about 1:1.

21. An active heterodimeric enzyme prepared according to the method of claim 1.

22. The active heterodimeric enzyme of claim 21, wherein each of the first species and the second species is independently within an Arabidopsis genus, a Petunia genus, a Prunus genus, or a Solanum genus, wherein the first species and the second species are not the same.

23. The active heterodimeric enzyme of claim 21, wherein the first species is Petunia hybrida or Solanum lycopersicum and the second species is not within the Arabidopsis genus.

24. The active heterodimeric enzyme of claim 23, wherein:
(i) the first species is Arabidopsis thaliana and the second species is Prunus dulcis, Petunia hybrida, or Solanum lycopersicum;
(ii) the first nucleic acid sequence encodes an Arabidopsis thaliana BSα subunit and the second nucleic acid sequence encodes a Prunus dulcis BSβ subunit; or
(iii) the first nucleic acid sequence encodes an Arabidopsis thaliana BSα subunit and the second nucleic acid sequence encodes a Petunia hybrida BSβ subunit.

25. The active heterodimeric enzyme of claim 21, wherein the first nucleic acid sequence encodes a Solanum lycopersicum BSα and the second nucleic acid sequence encodes a Solanum lycopersicum BSβ.

26. (canceled)

27. The active heterodimeric enzyme of claim 21, wherein the BSα subunit is or comprises a nucleotide sequence of SEQ ID NO: 3 or a nucleotide sequence having at least 50% identity to SEQ ID NO: 3 that encodes a BSα subunit.

28. The active heterodimeric enzyme of claim 21, wherein the BSβ subunit is or comprises a nucleotide sequence of SEQ ID NO: 4 or a nucleotide sequence having at least 50% identity to SEQ ID NO: 4 that encodes a BSβ subunit.

29. The active heterodimeric enzyme of claim 27, wherein the BSα subunit is or comprises SEQ ID NO: 101, SEQ ID NO: 103, or SEQ ID NO: 105.

30. The active heterodimeric enzyme of claim 28, wherein the BSβ subunit is or comprises SEQ ID NO: 102, SEQ ID NO: 104, or SEQ ID NO: 106.

31. (canceled)

32. A transgenic plant comprising:
a first heterologous nucleic acid sequence encoding a benzaldehyde synthase alpha (BSα) subunit and a second heterologous nucleic acid sequence encoding a benzaldehyde synthase beta (BSβ) subunit, wherein one or both of the first and second nucleic acid sequences is operably linked to a regulatory element for directing expression of the first and/or second nucleic acid sequences; and
wherein:
(i) the transgenic plant overexpresses at least the BSβ subunit,
(ii) the first heterologous nucleic acid is from a first species and the second heterologous nucleic acid is from a second species, or
(iii) both (i) and (ii).

33. The transgenic plant of claim 32, wherein both the BSα subunit and the BSβ subunit are overexpressed in the transgenic plant.

34. The transgenic plant of claim 32, wherein the first nucleic acid sequence comprises a nucleotide sequence of SEQ ID NO: 3, or a nucleotide sequence that is at least 50% identity to SEQ ID NO: 3 and encodes a BSα subunit.

35. The transgenic plant of claim 34, wherein the first nucleic acid sequence is or comprises SEQ ID NO: 101, SEQ ID NO: 103, or SEQ ID NO: 105.

36. The transgenic plant of claim 32, wherein the second nucleic acid sequence is or comprises a nucleotide sequence of SEQ ID NO: 4, or a nucleotide sequence that is at least 50% identity to SEQ ID NO: 4 and encodes a BSβ subunit.

37. The transgenic plant of claim 36, wherein the second nucleic acid sequence is or comprises SEQ ID NO: 102, SEQ ID NO: 104, or SEQ ID NO: 106.

38. The transgenic plant of claim 32, wherein the first nucleic acid sequence encodes SEQ ID NO: 1, SEQ ID NO: 5, SEQ ID NO: 6, SEQ ID NO: 7 or a functional fragment or homolog of SEQ ID NO: 1, SEQ ID NO: 5, SEQ ID NO: 6, or SEQ ID NO: 7.

39. The transgenic plant of claim 32, wherein the second nucleic acid sequence encodes SEQ ID NO: 2, SEQ ID NO: 8, SEQ ID NO: 9, SEQ ID NO: 10, or a functional fragment or homolog of SEQ ID NO: 2, SEQ ID NO: 8, SEQ ID NO: 9, or SEQ ID NO: 10.

40. The transgenic plant of claim 32, wherein:
the first nucleic acid sequence encodes SEQ ID NO: 1, SEQ ID NO: 5, SEQ ID NO: 6, SEQ ID NO: 7 or a functional fragment or homolog of SEQ ID NO: 1, SEQ ID NO: 5, SEQ ID NO: 6, or SEQ ID NO: 7; and
the second nucleic acid sequence encodes SEQ ID NO: 2, SEQ ID NO: 8, SEQ ID NO: 9, SEQ ID NO: 10, or a functional fragment or homolog of SEQ ID NO: 2, SEQ ID NO: 8, SEQ ID NO: 9, or SEQ ID NO: 10.

41. The transgenic plant of claim 39, wherein the transgenic plant is Petunia hybrida, Nicotiana benthamiana, or Prunus dulcis.

42. The transgenic plant of claim 39, wherein the first nucleic acid sequence is from a first species and the second nucleic acid sequence is from a second species.

43. The transgenic plant of claim 42, wherein each of the first species and the second species is independently within an Arabidopsis genus, a Petunia genus, a Prunus genus, or a Solanum genus, wherein the first species and the second species are not the same.

44. The transgenic plant of claim 43, wherein the first species is Arabidopsis thaliana and the second species is Prunus dulcis, Petunia hybrida, or Solanum lycopersicum.

45. The transgenic plant of claim 42, wherein benzaldehyde production of the transgenic plant is at least 4-fold greater than benzaldehyde production in a corresponding wild-type plant.

46. The transgenic plant of claim 42, wherein benzaldehyde production of the transgenic plant is at or near 7.8-fold greater than benzaldehyde production in a corresponding wild-type plant.

47. The transgenic plant of claim 39, wherein the regulatory element comprises a tissue-specific promoter for directing expression of the first and/or second nucleic acid sequence in the cells of a leaf, a root, a flower, a developing ovule or a seed of the transgenic plant.

48. The transgenic plant of claim 37, wherein both the first species and the second species are derived from a NAD(P)-binding Rossmann-fold superfamily.
